SEQUENCE LISTING (102 sequences)

SEQ ID NO: 1 - DNA, 21 bp, artificial sequence
  primer
gaccuaccugugcuacgaatt

SEQ ID NO: 2 - DNA, 21 bp, Artificial Sequence
  primer
gcaguaugcucccgaucaatt

SEQ ID NO: 3 - DNA, 18 bp, Artificial Sequence
  forward primer
ggaattcggcacgaggac

SEQ ID NO: 4 - DNA, 20 bp, Artificial Sequence
  primer
ggtcctcagtggatgtaggc

SEQ ID NO: 5 - DNA, 21 bp, Artificial Sequence
  primer
tggactgcagatactgctgct

SEQ ID NO: 6 - DNA, 18 bp, Artificial Sequence
  primer
ttgccggccacaaccctt

SEQ ID NO: 7 - DNA, 20 bp, Artificial Sequence
  primer
agccttgtctgggtcccatc

SEQ ID NO: 8 - DNA, 17 bp, Artificial Sequence
  forward primer
agcctcgcctttgccga

SEQ ID NO: 9 - DNA, 15 bp, Artificial Sequence
  reverse primer
ctggtgcctggggcg

SEQ ID NO: 10 - DNA, 20 bp, Artificial Sequence
  forward primer
tgacatcccccgcatcctgg

SEQ ID NO: 11 - DNA, 21 bp, Artificial Sequence
  reverse primer
catgtgctggtcattgagcag

SEQ ID NO: 12 - DNA, 26 bp, Artificial Sequence
  forward primer
cacacatattcacttccaactttaac

SEQ ID NO: 13 - DNA, 18 bp, Artificial Sequence
  reverse primer
gtccaggcgctccacttc

SEQ ID NO: 14 - DNA, 17 bp, Artificial Sequence
  forward primer
ggccgaggacccgaagg

SEQ ID NO: 15 - DNA, 20 bp, Artificial Sequence
  reverse primer
ttctgacacaggctgcgaag

SEQ ID NO: 16 - DNA, 19 bp, Artificial Sequence
  forward primer
atgtccgcgcagaacagaa

SEQ ID NO: 17 - DNA, 21 bp, Artificial Sequence
  reverse primer
caacttcatcctgaatctcct

SEQ ID NO: 18 - DNA, 22 bp, Artificial Sequence
  forward primer
ctttgacgagactctacagaag

SEQ ID NO: 19 - DNA, 21 bp, Artificial Sequence
  reverse primer
aaagagcacgcagaggtccag

SEQ ID NO: 20 - DNA, 20 bp, Artificial Sequence
  forward primer
aggcaggaagacctggcaga

SEQ ID NO: 21 - DNA, 20 bp, Artificial Sequence
  reverse primer
gggtacagtgcagacgaatc

SEQ ID NO: 22 - DNA, 21 bp, Artificial Sequence
  forward primer
aaacctgcagatgaccaagac

SEQ ID NO: 23 - DNA, 21 bp, Artificial Sequence
  reverse primer
agtcatcttttggcttggaag

SEQ ID NO: 24 - DNA, 20 bp, Artificial Sequence
  forward primer
agagagagcaccatttactg

SEQ ID NO: 25 - DNA, 20 bp, Artificial Sequence
  reverse primer
atatcccaggagtacaccca

SEQ ID NO: 26 - DNA, 21 bp, Artificial Sequence
  forward primer
ctcacccaggaggggagaatc

SEQ ID NO: 27 - DNA, 23 bp, Artificial Sequence
  reverse primer
cgatagcaattgccctgaaatcc

SEQ ID NO: 28 - DNA, 22 bp, Artificial Sequence
  forward primer
gcaaaagaagttaacagctgag

SEQ ID NO: 29 - DNA, 21 bp, Artificial Sequence
  reverse primer
gttggttgtccagcagtgact

SEQ ID NO: 30 - DNA, 20 bp, Artificial Sequence
  forward primer
tcaacctcgactcagcgctg

SEQ ID NO: 31 - DNA, 20 bp, Artificial Sequence
  reverse primer
gatgtggaaggcatcctgca

SEQ ID NO: 32 - DNA, 18 bp, Artificial Sequence
  forward primer
ttgttgtcagaggcccca

SEQ ID NO: 33 - DNA, 20 bp, Artificial Sequence
  reverse primer
tccttccgcctgagcttctt

SEQ ID NO: 34 - DNA, 22 bp, Artificial Sequence
  forward primer
ggaagatcatcagtcaaggaag

SEQ ID NO: 35 - DNA, 22 bp, Artificial Sequence
  reverse primer
cccaagtatgatcaagaatagc

SEQ ID NO: 36 - DNA, 20 bp, Artificial Sequence
  forward primer
tggctgctaccccactcctg

SEQ ID NO: 37 - DNA, 21 bp, Artificial Sequence
  reverse primer
gccctatttgctggatcatcc

SEQ ID NO: 38 - DNA, 20 bp, Artificial Sequence
  forward primer
aatcggctggcgcaacgtca

SEQ ID NO: 39 - DNA, 20 bp, Artificial Sequence
  reverse
ctcgtaggtcttcaccatcc

SEQ ID NO: 40 - DNA, 20 bp, Artificial Sequence
  forward primer
ctcagctccagtggaaccag

SEQ ID NO: 41 - DNA, 20 bp, Artificial Sequence
  reverse primer
aggatcccgttcaccatcac

SEQ ID NO: 42 - DNA, 21 bp, Artificial Sequence
  forward primer
cacctgcaagctctatgccat

SEQ ID NO: 43 - DNA, 19 bp, Artificial Sequence
  reverse primer
caaggggagcagcagaagg

SEQ ID NO: 44 - DNA, 24 bp, Artificial Sequence
  forward primer
aaatccatctggcataaatgatga

SEQ ID NO: 45 - DNA, 20 bp, Artificial Sequence
  reverse sequence
ctccatttcctgccttagcc

SEQ ID NO: 46 - DNA, 20 bp, Artificial Sequence
  forward sequence
gggaatgctgctggagatag

SEQ ID NO: 47 - DNA, 24 bp, Artificial Sequence
  reverse sequence
tcaaaccttgagttgaattccata

SEQ ID NO: 48 - DNA, 18 bp, Artificial Sequence
  forward primer
tggtaccaggcctccaag

SEQ ID NO: 49 - DNA, 20 bp, Artificial Sequence
  reverse primer
ctgcggtggtagtcgttgtc

SEQ ID NO: 50 - DNA, 23 bp, Artificial Sequence
  forward sequence
gatgatgttaagaagttcaaacc

SEQ ID NO: 51 - DNA, 22 bp, Artificial Sequence
  reverse primer
atcttgcttcctcttgagtgca

SEQ ID NO: 52 - DNA, 22 bp, Artificial Sequence
  forward primer
gaggacataactctagaatctg

SEQ ID NO: 53 - DNA, 22 bp, Artificial Sequence
  reverse primer
gatttatgtctcctcttcttac

SEQ ID NO: 54 - DNA, 22 bp, Artificial Sequence
  forward sequence
gaaaatgcaagataagacgcag

SEQ ID NO: 55 - DNA, 20 bp, Artificial Sequence
  reverse primer
ctggtcctggagtttctgga

SEQ ID NO: 56 - DNA, 19 bp, Artificial Sequence
  forward primer
gctgcacctcagcgacaac

SEQ ID NO: 57 - DNA, 19 bp, Artificial Sequence
  rever primer
ctgagcctggagctggggt

SEQ ID NO: 58 - DNA, 22 bp, Artificial Sequence
  reverse sequence
gtcaaagtagagtcaacttcat

SEQ ID NO: 59 - DNA, 21 bp, Artificial Sequence
  forward primer
tgtggatagtctggataagct

SEQ ID NO: 60 - DNA, 19 bp, Artificial Sequence
  reverse primer
gcacatgctccttggtcca

SEQ ID NO: 61 - DNA, 20 bp, Artificial Sequence
  forward primer
tgattacctagaccgagggc

SEQ ID NO: 62 - DNA, 22 bp, Artificial Sequence
  reverse sequence
atgccatcagcaagaggtttgt

SEQ ID NO: 63 - DNA, 20 bp, Artificial Sequence
  forward sequence
atagcagtggcctgagaaag

SEQ ID NO: 64 - DNA, 20 bp, Artificial Sequence
  reverse sequence
ctctgacaccaggtgcatgg

SEQ ID NO: 65 - DNA, 20 bp, Artificial Sequence
  forward primer
atctcaggagaatcgttggt

SEQ ID NO: 66 - DNA, 22 bp, Artificial Sequence
  reverse sequence
agcagactgtgaagaagctgct

SEQ ID NO: 67 - DNA, 19 bp, Artificial Sequence
  forward sequence
ggcgatgactcggacccag

SEQ ID NO: 68 - DNA, 20 bp, Artificial Sequence
  reverse sequence
ccaaattcacccaagaagag

SEQ ID NO: 69 - DNA, 20 bp, Artificial Sequence
  forward sequence
tgtgacatcgtaaacatgag

SEQ ID NO: 70 - DNA, 22 bp, Artificial Sequence
  reverse sequence
ggaatgtaccactcatatgaag

SEQ ID NO: 71 - DNA, 22 bp, Artificial Sequence
  forward primer
ccagactccgattttgatggag

SEQ ID NO: 72 - DNA, 21 bp, Artificial Sequence
  reverse primer
gctcttctttcctcaggagtg

SEQ ID NO: 73 - DNA, 22 bp, Artificial Sequence
  forward primer
gcagaagaatggtacaaatcca

SEQ ID NO: 74 - DNA, 21 bp, Artificial Sequence
  reverse primer
ctgtaggtggcaatctcaatg

SEQ ID NO: 75 - DNA, 19 bp, Artificial Sequence
  forward primer
gcatcagcaagagcaacag

SEQ ID NO: 76 - DNA, 21 bp, Artificial Sequence
  reverse primer
tcagtacttcttgagccattc

SEQ ID NO: 77 - DNA, 21 bp, Artificial Sequence
  forward primer
gtcatgggatcagtggcttac

SEQ ID NO: 78 - DNA, 23 bp, Artificial Sequence
  forward primer
gcaagagttccttttcatcttgc

SEQ ID NO: 79 - DNA, 23 bp, Artificial Sequence
  forward primer
ctttcattataaacccgctatag

SEQ ID NO: 80 - DNA, 20 bp, Artificial Sequence
  forward primer
tcaccctgtgtcctttgcag

SEQ ID NO: 81 - DNA, 22 bp, Artificial Sequence
  forward primer
gaagactatttgctttccctgg

SEQ ID NO: 82 - DNA, 19 bp, Artificial Sequence
  reverse primer
ggaggggattcttgctcac

SEQ ID NO: 83 - DNA, 23 bp, Artificial Sequence
  forward primer
tatgcatgatttaccatctttgc

SEQ ID NO: 84 - DNA, 19 bp, Artificial Sequence
  forward primer
gttggctttggtccaccac

SEQ ID NO: 85 - DNA, 21 bp, Artificial Sequence
  reverse primer
tgaaaatagccacacatacgg

SEQ ID NO: 86 - DNA, 21 bp, Artificial Sequence
  forward primer
ctttctcttgccacagcagct

SEQ ID NO: 87 - DNA, 23 bp, Artificial Sequence
  forward primer
ccagcaaaatggaattatcttgt

SEQ ID NO: 88 - DNA, 21 bp, Artificial Sequence
  reverse primer
ctctccttcaatagctggctt

SEQ ID NO: 89 - DNA, 20 bp, Artificial Sequence
  forward primer
gcactcagctcactgtgctt

SEQ ID NO: 90 - DNA, 20 bp, Artificial Sequence
  forward primers
cagttggtccctttttcagc

SEQ ID NO: 91 - DNA, 21 bp, Artificial Sequence
  revererse primer
aacacatgccatcacaatgcc

SEQ ID NO: 92 - DNA, 21 bp, Artificial Sequence
  forward primer
tgcattgtttattcctcaggc

SEQ ID NO: 93 - DNA, 21 bp, Artificial Sequence
  reverse primer
gctttgtaaaagcaactgggt

SEQ ID NO: 94 - DNA, 20 bp, Artificial Sequence
  forward primer
caccagacatctttctcacc

SEQ ID NO: 95 - DNA, 21 bp, Artificial Sequence
  reverse primer
acgcatccaagtctgagttcc

SEQ ID NO: 96 - DNA, 20 bp, Artificial Sequence
  primer
gcatccggacaggcatccaa

SEQ ID NO: 97 - DNA, 21 bp, Artificial Sequence
  forward primer
gccatgcggacctgcgcttct

SEQ ID NO: 98 - DNA, 19 bp, Artificial Sequence
  reverse primer
ggccgtaaaagccacagag

SEQ ID NO: 99 - DNA, 21 bp, Artificial Sequence
  forward primer
ctggagccccagcttctcctg

SEQ ID NO: 100 - DNA, 21 bp, Artificial Sequence
  reverse sequence
gagatgaaccaagtgaccctg

SEQ ID NO: 101 - DNA, 26 bp, Artificial Sequence
  forward sequence
tgattacgacgccctatataaggagg

SEQ ID NO: 102 - DNA, 18 bp, Artificial Sequence
  reverse sequence
tagatgcgggcagcgaag

(19) United States
(12) Patent Application Publication
(10) Pub. No.: US 2018/0072793 A1
(43) Pub. Date: Mar. 15, 2018

(54) APOBEC3A CYTIDINE DEAMINASE INDUCED RNA EDITING

(71) Applicant: Health Research, Inc., Buffalo, NY (US)

(72) Inventors: Bora E. BAYSAL, Orchard Park, NY (US); Shraddha SHARMA, Williamsville, NY (US); Santosh K. PATNAIK, Buffalo, NY (US)

(21) Appl. No.: 15/564,984

(22) PCT Filed: Apr. 11, 2016
(86) PCT No.: PCT/US2016/026911

§ 371 Date: Oct. 6, 2017

Related U.S. Application Data
(60) Provisional application No. 62/145,056, filed on Apr. 9, 2015.

(51) Int. Cl. C07K 14/775; C12Q 1/68; C07K 14/57; C12N 15/11; C12N 9/78

(52) U.S. Cl. CPC C07K 14/775; C12Q 1/6883; C07K 14/57; C12P 21/06; C12N 9/78; C12Q 2600/136; C12Q 2600/156; C12N 15/11

(57) ABSTRACT

Provided are methods for identifying agents which can induce or inhibit C>U deamination in RNA driven by apolipoprotein B editing catalytic proteins. The method comprises contacting APOBEC3A or APOBEC3G with a suitable RNA substrate and determining the extent of C>U deamination under conditions which induce APOBEC driven C>U deamination.

### CROSS-REFERENCE TO RELATED APPLICATIONS

This application claims priority to U.S. Provisional Application No. 62/145,056, filed on Apr. 9, 2015, the disclosure of which is incorporated herein by reference.

### FIELD OF THE DISCLOSURE

This disclosure relates generally to the field of RNA editing and particularly to C>U deamination by apolipoprotein B editing catalytic (APOBEC) proteins.

### BACKGROUND OF THE INVENTION

RNA editing is a co- or post-transcriptional process that alters transcript sequences without any change in the encoding DNA sequence. Although various types of RNA editing have been observed in single cell organisms to mammals, base modifications by deamination of adenine to inosine (A>I), or cytidine to uracil (C>U) are the major types of RNA editing in higher eukaryotes. I and U are read as guanosine (G) and thymine (T) respectively by the cellular machinery during mRNA translation and reverse transcription. RNA editing can therefore alter amino acid sequences, thereby modifying and diversifying protein functions. Aberrant RNA editing is linked to neuropsychiatric diseases such as epilepsy and schizophrenia, and chronic diseases such as cancer.

RNA-dependent ADAR1, ADAR2 and ADAR3 adenosine deaminases, and APOBEC1 cytidine deaminase are the only known RNA editing enzymes in mammals. RNA sequencing studies suggest that A>I RNA editing affects hundreds of thousands of sites, though most of A>I RNA edits occur at a low level and in non-coding intronic and untranslated regions, especially in the context of specific sequences such as Alu elements. A>I editing of protein-coding RNA sequences at a high level (>20%) is rare and thought to occur predominantly in the brain. Unlike A>I editing catalyzed by adenosine deaminases5, the prevalence and level of C>U RNA editing in different types of cells, and its enzymatic basis and regulation are poorly understood. The activation-induced deaminase (AID), apolipoprotein B editing catalytic polypeptide-like (APOBEC) family, and cytidine deaminase (CDA) proteins of mammals harbor the cytidine deaminase motif for hydrolytic deamination of C to U. CDA is involved in the pyrimidine salvaging pathway. While AID causes C>U deamination of DNA, multiple studies have failed to identify any RNA editing activity for this protein. Humans have 10 APOBEC genes (APOBEC1, 2, 3A-D, 3F-H and 4). APOBEC3 proteins can deaminate cytidines in single-stranded (ss) DNA, and although the APOBEC proteins bind RNA, C>U deamination of RNA is known for only APOBEC1, with apolipoprotein B (APOB) mRNA as its physiological target. C>U RNA editing alters hundreds of cytidines in chloroplasts and mitochondria of flowering plants, but the underlying deaminating enzymes are unknown. The extent, regulation and enzymatic basis of RNA editing by cytidine deamination are incompletely understood.

### SUMMARY OF THE DISCLOSURE

In this disclosure, we demonstrate transcripts of hundreds of genes undergo site-specific C>U RNA editing in macrophages during M1 polarization and in monocytes in response to hypoxia and/or interferons. This editing alters the amino acid sequences for scores of proteins, including many that are involved in pathogenesis of viral diseases. APOBEC3A, which is known to deaminate cytidines of single-stranded DNA and to inhibit viruses and retrotransposons, mediates this RNA editing. Amino acid residues of APOBEC3A (also referred to herein as A3A or C3A) that are known to be required for its DNA deamination and antiretrotransposition activities were also found to affect its RNA deamination activity. Our study demonstrates the cellular RNA editing activity of a member of the APOBEC3 family of innate restriction factors and expands the understanding of C>U RNA editing in mammals.

Based on the present findings, this disclosure provides compositions and methods for identifying agents that can affect (inhibit or enhance) the C to U deamination of RNA by APOBEC3A.

We also demonstrate that another APOBEC protein, the APOBEC3G (also referred to herein as A3G or C3G) is also capable of C>U RNA editing and provide compositions and methods for identifying agents that can affect (inhibit or enhance) the C to U deamination of RNA by APOBEC3G.

This disclosure provides a method for identifying agents that enhance or inhibit C>U deamination in a RNA substrate comprising providing a RNA substrate which contains a motif that contains a C that can undergo deamination to U; contacting the RNA substrate with a apolipoprotein B editing catalytic (APOBEC) protein (such as APOBEC3A or APOBEC3G) in the presence or absence of test agents under conditions such that C>U deamination occurs; and determining the extent of C>U deamination and identifying agents in the presence of which either an increase or decrease of deamination is observed as compared to deamination in the absence of the agent. The assay can be done in in vitro systems using purified APOBEC proteins or using cell lysates.

The disclosure also provides a method for identifying agents that enhance or inhibit C>U deamination in a RNA molecule comprising providing cells which express or overexpress APOBEC3A or APOBEC3G; in the presence or absence of test agents, optionally exposing the cells to conditions (such as hypoxia and/or interferons) under which the cells will carry out APOBEC3A driven C>U deamination of RNA or APOBEC3G driven C>U deamination of RNA; and determining the extent of C>U deamination in RNA to identify agents that induce or inhibit C>U deamination in RNA.

### BRIEF DESCRIPTION OF THE FIGURES

FIG. 1. SDHB c.136C>U RNA editing in interferon-treated monocyte enriched peripheral blood mononuclear cells (MEPs) and M1 macrophages (a) Mean and its standard error (n=3) are shown on left for editing levels in MEPs optionally treated with IFN1 (600 U per ml), IFNγ (200 U per ml) and hypoxia (1% O₂) for 24 hours. The additive induction of SDHB c.136C>U RNA editing by the interferons and hypoxia is also depicted on right. Matched MEPs of seven individuals were cultured under normoxia or hypoxia with 0, 300 or 1,500 U per ml IFN1 for 24 hours. Mean and its standard error (n=7) for editing levels in the cells are shown. Editing level in cells treated with both hypoxia and IFN1 was higher than in cells treated with only hypoxia or IFN1 (Wilcoxon test P<0.02, for both concentrations of IFN1). (b) M1 and M2 macrophages were generated from unpolarized M0 macrophages derived from CD14+ monocytes isolated from peripheral blood of three individuals. Mean and range (n=3) of expression of genes for markers of M1 and M2 polarization and SDHB c.136C>U editing levels in the cells are depicted. Gene expression was quantified by RT-PCR and normalized to that of ACTB.

FIG. 2. RNA editing in MEPs and macrophages (a) Mean and range of RNA editing levels (%) at sites identified by comparing transcriptome sequences of three pairs of hypoxic and normoxic MEPs, or M1 and M2 macrophages for differential RNA editing under hypoxia or M1 polarization. (b) Cumulative frequency plots of mean editing levels and fold-change effects of hypoxia or M1 polarization on editing level, by type of RNA editing. Fold-change values were estimated with the inverted beta-binomial test and their absolute values are capped at 10⁴. (c) Distributions for editing sites in coding RNAs of gene feature and effect of editing on amino acid coding, by type of RNA editing. (d) Logos indicating sequence conservation and nucleotide frequency for sequences bearing C>U editing sites (at position 0) with a higher editing level in hypoxic compared to normoxic MEPs (n=206) or M1 compared to M2 macrophages (n=122); mean and 95% CI of relative entropy values are also plotted. (e) Stem-loop structure in SDHB RNA with the c.136C>U editing site underlined and 5 b palindromes forming the stem indicated. Histograms depict the distributions of flanking palindrome length by sequence at −3 to 0 positions for the sites whose sequence logos are shown in d. (f) Effect of hypoxia or M1 polarization on transcript levels of genes that are expressed in MEPs or macrophages and code for ADAR and cytidine deaminase enzymes and some markers of M1 (FCER1A, MRC1) or M2 (CCL2, IL8) macrophage polarization. Mean and range (n=3) are shown; ns, not significant (FDR≧0.05, edgeR likelihood ratio test); #, not expressed; genes not marked ns or # are differentially expressed with FDR<0.05.

FIG. 3. C>U RNA editing induced in MEPs and monocytes by hypoxia and IFN1. (a) Site-specific C>U RNA editing for 19 genes of MEPs of three individuals was quantified by Sanger sequencing of RT-PCR products. MEPs were optionally treated with hypoxia and/or 600 U per ml IFN1 for 24 hours. (b) Editing of the sites was also similarly examined in hypoxia- and IFN1-treated MEPs of another three individuals, and in lymphocytes and CD14+ monocytes isolated from the MEPs. Because of absent or low gene expression, a C1QA RT-PCR product could not be obtained for any of the three lymphocyte isolates. Sanger sequence chromatograms for the three monocyte and two of the lymphocyte isolates are shown in FIG. 11c. Site-specific C>U RNA editing in the monocytes and lymphocytes for 12 other genes is depicted in FIG. 11b. Mean and its standard error (n=3) are shown in both panels. The detection limit for editing (5% level) is indicated. Samples without detectable editing were assigned a value of 3.8%. (c) SDHB and SIN3A protein levels in whole cell lysates (20 μg protein) of monocytes isolated from normoxic and hypoxic MEPs of a separate set of three donors. Non-specific signals of the Western blots are indicated by asterisk (*).

FIG. 4. Association of APOBEC3A gene expression with SDHB c.136C>U RNA editing in tumor samples of the Cancer Genome Atlas (a) C>U RNA editing was estimated from RNA sequencing data for primary head and neck squamous cell carcinoma (HNSC, n=298) and lung adenocarcinoma (LUAD, n=220), and secondary skin cutaneous melanoma (SKCM, n=187) tumors. Editing levels at all 213 C-bearing positions along SDHB ORF are plotted for every tumor. Mean levels at the positions (black), the c.136C site (red), and known C/T single nucleotide polymorphism sites (green) are indicated. Inset shows SDHB c.136C>U editing levels, and their mean and standard deviation for tumors identified as positive for the editing. (b) Tukey plots of expression of some APOBEC3 (A3), and hypoxia- (LDHA, PGK1) and macrophage-associated (CD14, MRC1) genes among SDHB c.136C>U editing-positive and -negative tumors. Error bars denote 25th percentile−1.5× inter-quartile range (IQR) and 75th percentile+1.5×IQR values. Group-sizes are noted in the legend. * FDR<0.05 (edgeR exact test for differential expression). In (b), for each set from left to right are shown for C>U− and +, HNSC, LUAD, and SKCM.

FIG. 5. APOBEC3A induces C>U RNA editing in 293T transfectants. (a) Immunoblots showing APOBEC3A (A3A), APOBEC3G (A3G) and CDA proteins in whole cell lysates (20 μg protein) of 293T cells transiently transfected with an empty vector (Ctrl., control) or DNA constructs for expression of A3A, A3G or CDA proteins. (b) SDHB c.136C>U RNA editing in the 293T transfectants, which were optionally treated with hypoxia and/or 600 U per ml type I interferon (IFN1). Mean and range for n=3 are shown. (c) Estimation of site-specific C>U RNA editing by Sanger sequencing of RT-PCR products for 30 genes in the transfectants (n=1). The detection limit for editing (5% level) is indicated. Samples without detectable editing were assigned a value of 3.8%. Chromatograms for 19 genes are shown in Supplementary FIG. 5. Chromatograms of good quality could not be obtained for C1QA and TMEM179B for the A3G and CDA transfectants, and for the GPR160 site for the normoxic A3A transfectant. (d) Chromatograms of genomic DNA (gDNA) and cDNA PCR products of normoxic A3A transfectants indicating C>U RNA editing without C>T genomic change at positions marked with * for ASCC2, SDHB and TMEM109. Immunoblots showing ASCC2, SDHB and TMEM109 proteins in whole cell lysates (20 μg protein) of control or A3A transfectants on the right indicate reduced protein expression in association with A3A-induced stop codons in RNA. Only a single band of signal, which corresponded to a protein of full length, was seen in all three immunoblots.

FIG. 6. Knock-down of APOBEC3A (A3A) reduces C>U RNA editing in M1 macrophages (a) A3A and APOBEC3G (A3G) gene expression in M1 macrophages that were transfected with a non-specific (Ctrl.) or either one (1, 2) or equimolar mix (1+2) of two A3A-specific siRNAs at 100 nM concentration. Gene expression measurements are normalized to that for ACTB. (b) Immunoblot for A3A protein (23 kDa) of whole cell lysates (10 μg protein) of two of each set of three replicate transfectants. Non-specific signals are indicated by an asterisk (*). The signal for calnexin, a house-keeping protein, indicates total protein. (c) SDHB c.136C>U RNA editing levels in the siRNA transfectants which are determined by RT-qPCR. (d) Sanger sequence chromatogram traces of amplified cDNA fragments indicating reduced site-specific RNA editing for five other genes in A3A-specific siRNA 1 compared to Ctrl. transfectants. Mean and range (n=3) are shown for a and c.

FIG. 7. Activity of APOBEC3A (A3A) mutants in 293T transfectants (a) A3A protein level in whole cell lysates (20 μg protein) of cells transfected with an empty vector (Ctrl.) or expression constructs for wild-type (WT) A3A or its C101S, E72D or P134A variants. (b) Cytidine deamination activity of the transfectant lysates was examined in an in vitro reaction with a 5′ fluorescent dye-labeled ssDNA substrate of 40 bases (b). C>U deamination of the single cytidine residue of the substrate at position 23 followed by deglycosylation of the uridine and subsequent cleavage of the product at the abasic site was evaluated by electrophoresis of reactions of one hour duration on a polyacrylamide gel, whose fluorographic image is shown. (c) SDHB c.136C>U RNA editing in the transfectants. (d) Retrotransposition of a human LINE-1 element in a separate set of 293T transfectants. Retrotransposition, relative to the Ctrl. transfectant, was assessed with a luciferase reporter-based assay and is quantified as the ratio of firefly and Renilla luciferase activities. Mean and range (n=3) are shown for c and d.

FIG. 8. In vitro cytidine deamination of SDHB RNA and ssDNA by APOBEC3A (a) c.136C>U editing of an ˜1.1 kb exogenous SDHB ORF RNA by whole cell lysates of control or APOBEC3A 293T transfectants. Duration of the deamination reactions and amount of lysate protein in them are noted. For some reactions, lysates were pre-heated at 85° C. for 15 minutes. (b) c.136C>U editing of the RNA by 10 μM purified C-His₆-tagged APOBEC3A protein. The reactions had 180 amole SDHB RNA and 100 nM ZnCl₂. (c) Sanger sequence chromatogram traces of PCR amplified products of SDHB deamination reactions that had either 180 amole of ˜1.1 kb SDHB RNA or 100 amole of SDHB ssDNA of 120 b as substrate. APOBEC3A protein was present in the +reactions at 5 and 20 μM in the reaction with RNA and DNA substrate, respectively. Reactions for b and c conducted for two hours at 37° C. Mean and range (n=3) are shown in a and b.

FIG. 9. Hypoxia and interferon 1 (IFN1) induce C>U RNA editing in monocyte-enriched peripheral blood mononuclear cells (MEPs). Sanger sequence chromatograms of RT-PCR products for 19 genes for which site-specific C>U RNA editing was validated using MEPs of one individual. Cells were optionally treated with hypoxia and/or 600 U per ml IFN1 for 24 hours. Black flags on chromatograms indicate the C>U RNA-edited positions.

FIG. 10. Sanger sequencing of genomic DNA at positions of C>U RNA editing sites. Genomic DNA of MEPs of two individuals (A and B) and 293T cells transiently transfected for expression of APOBEC3A transfectants, was examined by Sanger sequencing for C>T variation at positions of C>U RNA editing sites for 23 genes. Any C>U RNA editing at the sites was verified by sequencing RT-PCR products of the same cells (FIGS. 9 and 13). Sequence chromatograms with black flags indicating the C>U RNA-edited positions are shown. MEPs were optionally treated with hypoxia and 600 U per ml IFN1 for 24 hours. The 293T cells were optionally treated with hypoxia for 24 hours one day after transfection. Evidence for C>T variation is not seen in any chromatogram. Some genes were not examined for some of the samples. Some of the data depicted in this figure is also shown in FIG. 5d.

FIG. 11. C>U RNA editing by hypoxia and IFN1 in monocytes but not lymphocytes. (a) Dot plot of forward and side scatter values of a sample of MEPs cultured under hypoxia with 600 U per ml IFN1 for 24 hours, indicating the strategy used to isolate monocytes and lymphocytes from MEPs by flow cytometry based on light scattering and cell surface expression of CD14 protein. (b) Estimation of site-specific C>U RNA editing by Sanger sequencing of RT-PCR products for 12 genes in monocytes and lymphocytes isolated from hypoxic, IFN1-treated MEPs of three individuals. Editing levels for individual monocyte or lymphocyte samples and their means are shown. A level could not be calculated for FAM89B and RNH1 for some samples because of poor quality of Sanger sequencing. The detection limit for editing (5% level) is indicated. Samples without detectable editing were assigned a level of 3.8%. Information on site-specific C>U RNA editing in the samples for 19 other genes is shown in FIG. 3b. (c) Sanger sequence chromatograms of RT-PCR products for another 19 genes for which site-specific C>U RNA editing was validated. Data are shown for monocytes and lymphocytes of three individuals (A-C). These chromatograms are used to quantify the editing levels that are depicted in FIG. 3b. Black flags on chromatograms indicate the C>U RNA-edited positions. A C1 QA RT-PCR product for Sanger sequencing could not be obtained for lymphocytes.

FIG. 12. Effect of IFN1 on APOBEC3A, APOBEC3G and CDA gene expression in MEPs. MEPs of three individuals were optionally treated with 300 or 1,500 U per ml IFN1 for 24 hours under normoxia or hypoxia. SDHB c.136C>U RNA editing and expression of APOBEC3A, APOBEC3G and CDA (normalized to SDHB) were quantified by RT-PCR. Mean and range (n=3) of changes in gene expression and SDHB RNA editing relative to untreated cells are shown.

FIG. 13. C>U RNA editing in 293T cells transiently transfected for expression of APOBEC3A with or without p.C101S or p.E72D mutation, APOBEC3G or CDA. Sanger sequence chromatograms of RT-PCR products for 19 genes for which site-specific C>U RNA editing was validated. Cells were optionally treated with hypoxia for 24 hours one day after transfection. Black flags on chromatograms indicate the C>U RNA-edited positions. These chromatograms are used to quantify the editing levels that are depicted in FIG. 6a. Chromatograms of good quality could not be obtained for C1 QA for hypoxic APOBEC3A and CDA transfectants. The mutant APOBEC3A transfectants were examined for only five genes. Some of the data depicted in this figure is also shown in FIG. 5d.

FIG. 14. SDHB c.136C>U RNA editing in 293T cells co-transfected with expression constructs for APOBEC3A and SDHB open reading frames. 293T cells were transiently transfected with the plasmid DNAs. An empty vector was used for cells that did not receive the APOBEC3A plasmid. Reverse transcription reactions were performed with or without reverse transcriptase (R7), and the products were used as template in allele-specific PCR to quantify editing of either exogenous or both exogenous and endogenous SDHB transcripts. Mean and range (n=3) are shown.

FIG. 15. Effect of freeze/thaw and cell density on SDHB c.136C>U RNA editing. (a) CD14⁺ monocytes, isolated from PBMCs of one donor using immunomagnetic beads and stored frozen in RPMI-1640 medium with 36% v/v fetal bovine serum and 10% v/v dimethyl sulfoxide at −80° C., were thawed and cultured at indicated density for an hour and then optionally treated with hypoxia for a day. Mean and range (n=3) of editing levels in normoxic and hypoxic cells are shown. Significant induction of SDHB RNA editing by hypoxia (>2-fold, compared to normoxia) is not noticeable. (b) In freshly isolated MEPs, a high level of editing under hypoxia was more consistently observed with a cell density above 20 million/ml. The data that is plotted was generated in multiple experiments that had a total of 78 cultures of MEPs isolated from a total of 33 donors. Seventy-six of the cultures were paired; i.e., cells of a specific donor and at a specific density were cultured under either hypoxia or normoxia for a day.

FIG. 16. Correlation of SDHB c.136C>U RNA editing level measurements obtained by allele-specific RT-PCR and Sanger sequencing. The scatterplot shows estimates of editing level determined by both RT-PCR and Sanger sequencing of amplified cDNA for 22 samples of normoxic or hypoxic MEPs. Values of the Pearson correlation coefficient (r) and slope (m) of the linear regression line (black; least squares fitting technique), and their 95% confidence intervals, and the line of identity (gray) are also depicted.

FIG. 17. Scans of films of immunoblotting assays whose results are shown in FIGS. 3c, 5d and 6b. (a) Cropped views of these scans are shown in FIG. 3c. For SIN3A, that figure shows only the signal at the position marked with an asterisk (*). (b) Cropped views of these scans, only for experiment (Expt.) B, are shown in FIG. 5d. (c) Cropped views of these scans are shown in FIG. 6b. That figure shows the two signals at positions marked with an asterisk (*) for lanes that are marked here as 2-3, 5-8 and 13-14. Lanes 1-3, 4-6, 7-9 and 13-15 respectively had protein lysates of the biological triplicates of Ctrl., 1, 2 and 1+2 siRNA transfectants. Molecular weight (MW) markers and the protein(s) being detected are noted in all three panels of the figure.

FIG. 18. Read counts in raw and processed RNA sequencing data

FIG. 19. Mapping of RNA sequencing data with the Subread subjunc aligner

FIG. 20. Mapping of RNA sequencing data with the TopHat2 aligner

FIG. 21. Number of candidate sites along different steps of analysis of pileups of Subread-aligned RNA sequencing reads for identification of differentially RNA-edited sites showing separate analyses of RNA sequencing data of MEPs and macrophages

FIG. 22. Summary of Supplementary Data 1 for genomic feature and effect on translation codon of RNA editing at positions for which the editing level was differentially affected by hypoxia or macrophage polarization as annotated by ANNOVAR

FIG. 23. Enrichment for ontologies of genes for sites with C>U RNA editing differentially affected by hypoxia or M1 macrophage polarization. Gene set enrichment analyses, for sets with at least two genes, were performed with PANTHER 9.0

FIG. 24. Genes differentially expressed between tumor samples of the Cancer Genome Atlas (TCGA) that are positive positive or negative for SDHB c.136C>U editing in all three cancers. Only genes with absolute loge fold-change >0.5 were considered; genes are ordered by decreasing mean of the three loge fold-change values

FIG. 25. Differential expression of genes coding for known RNA editing and cytidine deaminase enzymes following hypoxia treatment of MEPs, M1 (vs. M2) macrophage polarization, or between SDHB c.136C>U editing-positive and -negative TCGA tumor samples. Loge fold-change expression values are shown for genes identified as differentially expressed in hypoxic vs. normoxic MEP, M1 vs. M2 macrophage, or SDHB c.136C>U editing-positive vs. −negative cancer tumor tissue comparisons; ns, statistically insignificant for differential expression (FDR≧0.05; see Methods); NE, identified as not expressed (see Methods).

FIG. 26. Sequences of DNA oligonucleotides used as PCR or sequencing primers. Oligonucleotide used as a sequencing primer is indicated with an asterisk.

FIG. 27. Sequences of DNA oligonucleotides used for site-directed mutagenesis of APOBEC3A coding sequence. Reference sequence is NCBI RefSeq NM_145699.2

FIG. 28. Transient overexpression of APOBEC3G in 293T cells induces C>U RNA editing of host genes. Sanger sequencing of selected genes confirms site-specific C>U RNA editing by overexpressing A3G. EV=transfected with empty vector, A3G=transfected with expression plasmid for A3G.

FIG. 29. Salient characteristics of C>U RNA editing by APOBEC3G in 293T cells (A) Mean and range of editing level at the 712 sites identified as targets for A3G-mediated editing are shown for the three A3G transfectant samples. The sites are ordered by the mean editing level. (B) Logo indicating sequence conservation and base frequency for sequences bearing the editing sites (at position 0). (C) Histogram of lengths in bases of inverted repeat sequences flanking the editing sites.

FIG. 30. Site-directed mutagenesis of APOBEC3G shows requirement of both N- and C-terminal domain active catalytic site residues for site-specific RNA deamination. A. Sanger sequencing of selected genes shows that mutations in in conserved catalytical sites in N-terminal (C97S) and C-terminal (C291S) markedly decrease or abolish RNA editing (edited Cs are highlighted) whereas non-catalytic site mutants W94A and W127A has limited effect on RNA editing. B. Quantification of editing frequencies in selected sites is shown in B and C. In B, the bars from left to right for each set are: ITG1, PRPSAP2, RFX7, SCD, and TM7SF3. In C, the bars from left to right for each set are: GOLGA5 (R692X), KIAA1715, and MED1. Dotted line indicates the threshold (0.048) where RNA editing levels can be confidently measured by the Sequencher software.

FIG. 31 (A, B and C). Additional mutants confirm essential role of APOBEC3G N-terminal conserved catalytic residues for RNA editing. Mutations in N-terminal conserved catalytic residues H65R, E67Q, C97S and C100S markedly diminish or abolish RNA editing, whereas mutations in Vif-binding residues D128K and P129A has no effect on RNA editing levels. In C, the bars from left to right for each set are: KIAA1715, PRPSAP2, SCD, TM7SF3.

FIG. 32. APOBEC3G catalyzes site-specific RNA editing of KIAA1715 in vitro. KIAA1715 (uc002ukc.2) c.C751T undergoes RNA editing by 0.8 μM purified Myc-DDK-tagged A3G protein. In contrast, no obvious editing is noted at the corresponding site in the ssDNA sequence by Sanger Sequencing. The reactions were incubated for 2 hours with either 50 pg RNA (405 nt spanning c.632-c.1036) or 50 pg ssDNA (89 nt spanning c.68-c.772) in buffer containing the indicated reagents. All oligonucleotides were reverse transcribed, PCR amplified and sequenced by Sanger method after in vitro incubation.

### DETAILED DESCRIPTION OF THE INVENTION

The present disclosure is based on our identification of an enzyme and conditions which can induce C>U deamination. This disclosure provides methods and compositions to identify agents which can affect C>U deamination. Such agents may be useful for inhibition of C>U deamination or enhancing C>U deamination.

We have found that hypoxia (such as 1% 02) enhances the C>U editing of an exemplary RNA, e.g., SDHB RNA at c.136 in monocytes, with an editing level of ˜18% observed for monocyte-enriched PBMCs (MEPs) after 48 hours of hypoxia. Monocytes infiltrate tumors, atheromatous plaques, and sites of infection and inflammation, which are characterized by micro-environmental hypoxia. C>U RNA editing of SDHB may therefore represent a hypoxia-adaptive mechanism that may have implications for the pathogenesis of chronic inflammatory diseases.

To identify additional C>U RNA editing events in monocytes and monocyte-derived macrophages, we analyzed their whole transcriptome RNA sequences. We show that transcripts of hundreds of genes including those implicated in viral pathogenesis and Alzheimer's disease are targets of editing in monocytes and macrophages. We show that such editing is regulated by oxygen, interferons (IFN) and also during macrophage polarization. Most importantly, we demonstrate that APOBEC3A, which belongs to the APOBEC3 family of cytidine deaminases, is an RNA editing enzyme. These findings significantly expand our understanding of C>U RNA editing and open new avenues of inquiry on the role of APOBEC3 genes in viral and chronic diseases.

By transcriptome sequencing and analysis, we show here that transient overexpression of APOBEC3G in 293T cells causes site-specific C-to-U (C>U) RNA editing in 712 sites resulting in protein recoding of 217 cellular genes. APOBEC3G-mediated RNA editing causes recoding in CHMP4B, SIN3A, subunits of mediator complex MED (MED1, MED28), NFAT5, NMT1, RBM14 and MAPK1 that are known to be involved in HIV-1 replication. Site-directed mutagenesis studies show that conserved catalytic residues in both cytidine deaminase domains of APOBEC3G are required for RNA cytosine deamination. Purified C3G enzyme catalyzes site-specific RNA editing in vitro. These results demonstrate that APOBEC3G is a C>U RNA editing enzyme that may antagonize retroviral infection by mutating the transcripts of accessory host genes.

In one embodiment, the disclosure provides a method of identifying compounds which can induce or inhibit the C>U deamination of RNA comprising providing a substrate (RNA molecule) for the deamination and an enzyme that is capable of C>U deamination under conditions such that the enzyme will catalyze the C>U deamination of the RNA. The enzyme can be APOBEC3 or APOBEC3G. The enzyme may be provided in a purified or recombinant form such that the reaction can be carried out in a cell-free system. In one embodiment, the enzyme may be provided as a component of a cell lysate. In one embodiment, the enzyme may be provided in vivo. These enzymes are available commercially (such as from Origene).

The disclosure provides a method for screening a plurality of compounds or agents for their ability to induce or inhibit APOBEC3A and/or APOBEC3G driven C>U deamination in RNA substrate comprising the motif for the C>U deamination. The method can comprise contacting purified or recombinant APOBEC3A and/or APOBEC3G protein with the RNA substrate in an in vitro system in the presence or absence of the test compounds and determining C>U deamination. The method can comprise contacting cell lysates comprising APOBEC3A and/or APOBEC3G with the RNA substrate in the presence or absence of the test compounds or agents and determining C>U deamination. Increased C>U deamination identifies compounds or agents that enhance C>U deamination. Decreased C>U deamination identifies compounds or agents that inhibit C>U deamination. Identification of increased or decreased C>U deamination can be done relative to a control, which may be run in the absence of the enzyme, substrate or in the presence of enzymes or substrates that do not support C>U deamination.

In one embodiment, the disclosure provides a method for screening a plurality of compounds for their ability to induce or inhibit APOBEC3A driven C>U deamination in RNA comprising exposing whole cells, which express APOBEC3A and which comprise an RNA substrate, to conditions that induce APOBEC3A driven C>U deamination in the presence or absence of the test compounds and determining C>U deamination. Conditions that induce APOBEC3A driven C>U deamination can be hypoxia and/or interferons. For example, for monocytes, both hypoxia and/or interferons induce APOBEC3A driven C>U deamination. For macrophoages, interferons induces APOBEC3A driven C>U deamination.

In one embodiment, the method can comprise exposing whole cells, in which APOBEC3A and/or APOBEC3G has/have been overexpressed, and which comprise an RNA substrate, to test compounds and determining C>U deamination. Overexpression of APOBEC3A and/or APOBEC3G can be carried out in any cells, such as cell lines, such as 293T cells. The cells can then be processed for determining the level of C>U deamination. Increased C>U deamination identifies compounds or agents that enhance C>U deamination. Decreased C>U deamination identifies compounds or agents that inhibit C>U deamination.

In one embodiment, the disclosure provides a method for screening a plurality of compounds for their ability to induce APOBEC3A or APOBEC3G driven C>U deamination in a RNA molecule in the presence or absence of hypoxia and/or interferons comprising one or more of the following: i) testing the plurality of compounds for increasing deamination of C in isolated DNA molecule (such as a single stranded DNA molecule); ii) testing the positive compounds from i) for an enhancing effect on C>U deamination in isolated RNA molecule (such as, for example, SDHB); and iii) testing the positive compounds for enhancing effect on C>U RNA deamination from ii) in cell based assays, and then optionally in vivo systems. In one embodiment, step ii) could be eliminated with positive compounds from i) being directly tested for enhancing C>U RNA deamination in a cell based assay.

In one embodiment, the disclosure provides a method for screening a plurality of compounds for their ability to inhibit APOBEC3 driven C>U deamination in a RNA molecule in the presence or absence of hypoxia and/or interferons comprising performing one or more of the following: i) testing the plurality of compounds for reducing deamination C in isolated DNA molecule (such as a single stranded DNA molecule); ii) testing the positive compounds from i) for reducing C>U deamination in isolated RNA molecule; and iii) optionally testing the positive compounds for reducing C>U RNA deamination from ii) in cell based assays wherein the cells are exposed to hypoxia and/or interferons, and then optionally in vivo systems. In one embodiment, step ii) could be eliminated with positive compounds from i) being directly tested for reducing C>U RNA deamination in a cell based assay.

Determination of deamination of DNA or RNA can be carried out by methods known in the art. For example, DNA and RNA deamination can be determined by Sanger reaction or high throughput sequencing techniques. For DNA deamination, treatment with UDG glycosylase and alkaline treatment may be used to cleave the DNA molecule. For RNA deamination, radioactive primer extension and gel electrophoresis may be used. In one embodiment, allele specific qPCR may be used to measure RNA editing (Baysal et al., PeerJ., Sep. 10, 2013, 1:e152. doi: 10.7717/peerj.152. eCollection 2013); incorporated herein by reference).

Determination of C>U deamination can be carried out in a cell-free system. For example, isolated polynucleotide (polyribonucleotides or polydeoxyribonucleotides) may be used. In one embodiment, the length of the polynucleotides is at least 15. Thus, the substrates may be short polynucleotides or long RNA or single stranded DNA molecules. In one embodiment, the length is from 15 to 100 nucleotides and all integer lengths therebetween. In one embodiment, the length is from 30 to 50 nucleotides.

In vitro assays with purified APOBEC3A or APOBEC3G can be carried out by contacting APOBEC3A or APOBEC3G (such as 1-10 mM) with a suitable polynucleotide substrate (such as 2-2.5 pM full-length RNA or single-stranded DNA, in suitable buffers (such as 10 mM Tris (pH 8.0), 50 mM KCl and 10 or 100 uM ZnCl₂ with or without 10 mM 1,10-phenanthroline). The reactions can be incubated for suitable periods of time. For in vitro RNA-editing assay with transfectant cell lysate, reaction can be carried out at 37° C. for 4-11 h with RNA in a suitable buffer containing RNAse inhibitor (such as in 100 mM KCl, 10 mM HEPES (pH 7.4), 1 mM DTT and 1 mM EDTA). Cell-based assays can be performed 24-48 hours after transfection of cells (such as 293T cell line) with a mammalian expression vector containing APOBEC3A or APOBEC3G coding regions. This can be useful to achieve overexpression of the enzymes. Total RNA can be extracted and RT-qPCR is can be performed (such as by using a method described in Baysal et al. PeerJ:e152, incorporated herein by reference).

In one embodiment, the RNA substrate contains the motif CCAUCG with the underlined C targeted for editing. In one embodiment, variants of this motif can also be used which are single-nucleotide variations within the motif. In one embodiment, UC is the motif, and in one embodiment, CC is the motif with underlined C targeted for editing. In one embodiment, the RNA (polyribonucleotide substrate) contains stem-loop structures which contain the editable Cs in the loops. The stem loop size may be 4 nucleotides or more. In one embodiment, the RNA substrate comprises only one target C>U editing motif. In one embodiment, the RNA substrate comprises 2 or more target C>U editing motifs.

In one embodiment, the ss DNA substrate comprises the motif TC. In one embodiment, the ss DNA comprises the motif CC. The ss DNA substrate may comprise one or more target motifs.

For carrying out cell based assays, any type of cells may be used. For example, cells may be in vivo, or freshly isolated or primary or secondary cultures, or cell lines. In one embodiment, the cells are peripheral blood mononuclear cells (PBMCs). In one embodiment, the cells may be lymphocytes and monocytes. These cells may be purified from the blood by using routine methods (such as density gradients, flow cytometry and the like). The cells may be further purified as desired. For example, CD14 monocytes may be isolated using anti-CD14 antibody based methods employing magnets or flow cytometry. Alternatively, monocytes can be physically enriched by cold-aggregation of PBMCs, as described herein, or by plate adherence. We found that APOBEC3A-mediated RNA editing occurs primarily CD14 positive monocytes or monocyte-derived macrophages that are treated by interferons. The cells may be used as such, or may be transfected with vectors encoding APOBEC3A or APOBEC3G to result in overexpression of these enzymes. The cells may be exposed to hypoxia and/or interferons. The interferons can be IFN gamma and IFN1.

In one embodiment, the effect of various compounds may be tested after the cells are exposed to conditions of hypoxia and/or interferons. For example, hypoxic conditions may be created in culture by exposing cells to 10% or less O₂ (with 5% CO₂ and the rest nitrogen). In one embodiment, the O₂ is from 1 to 5%. In one embodiment, the O₂ is less than 1%. The cells may be exposed to the hypoxic conditions and/or interferons for desired lengths of time. For example, cells may be exposed for from 6 hours to 48 hours or more.

The interferons useful for the present methods include IFN gamma and IFN1. IFN1 is considered a ‘universal’ type I IFN. In one embodiment, the IFN1 comprises a hybrid of N-terminal IFNα-2 and C-terminal IFNα-1 produced in E. Coli. Useful range of interferon includes 50-500 U/ml for IFN gamma; 50 Um′ to 2,500 Um′ for IFN type 1 to induce APOBEC3A mediated RNA editing.

The agents identified by the methods of the present disclosure may be further tested for anti-tumor activity (such as those agents which inhibit C>U deamination) or for anti-viral activity (such as those agents which enhance C>U deamination activity).

The following examples are provided to further illustrate this invention.

### Example 1

### Methods

### Isolation and Culture of Cells

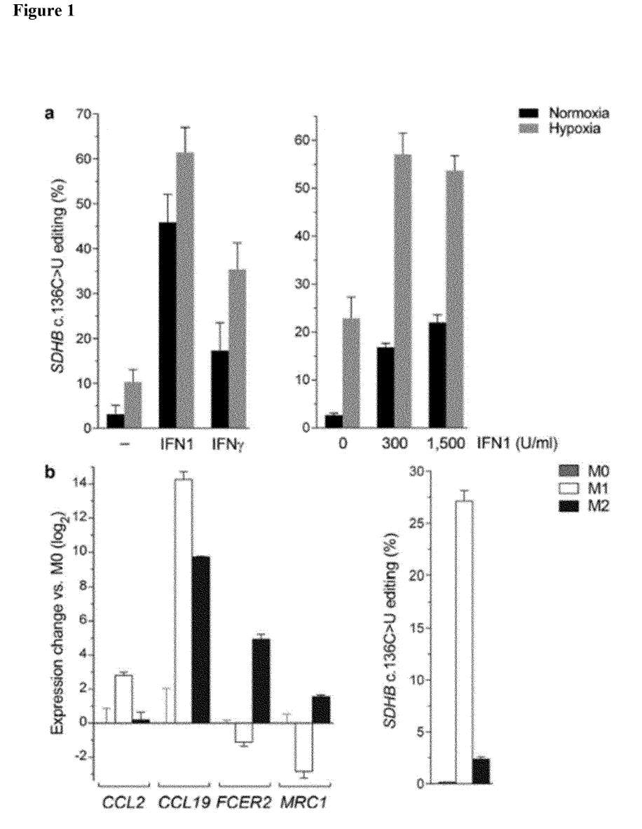

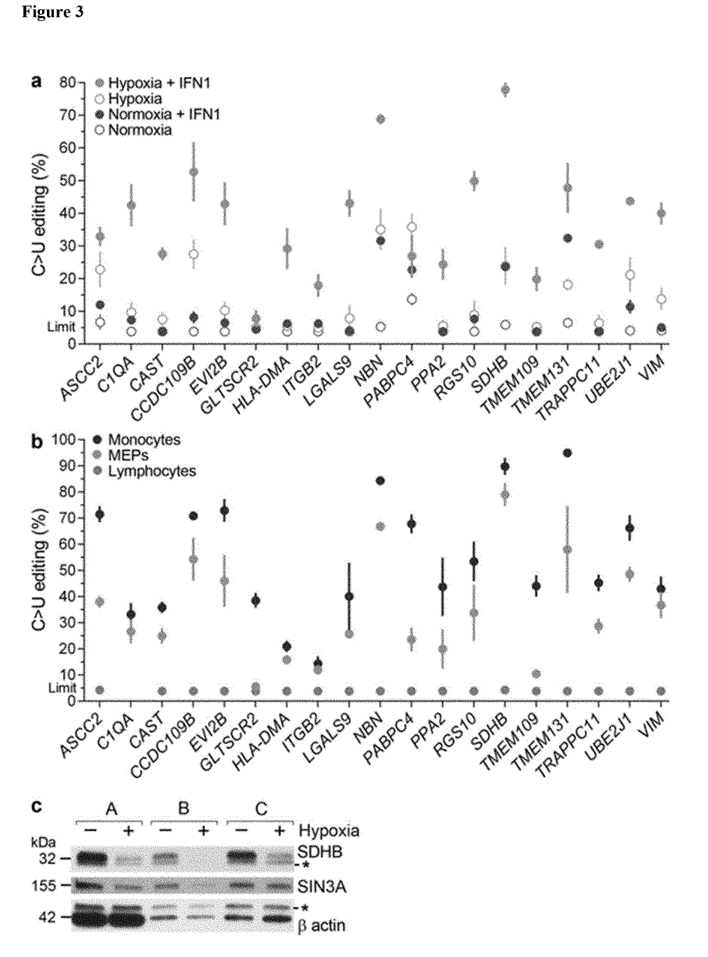

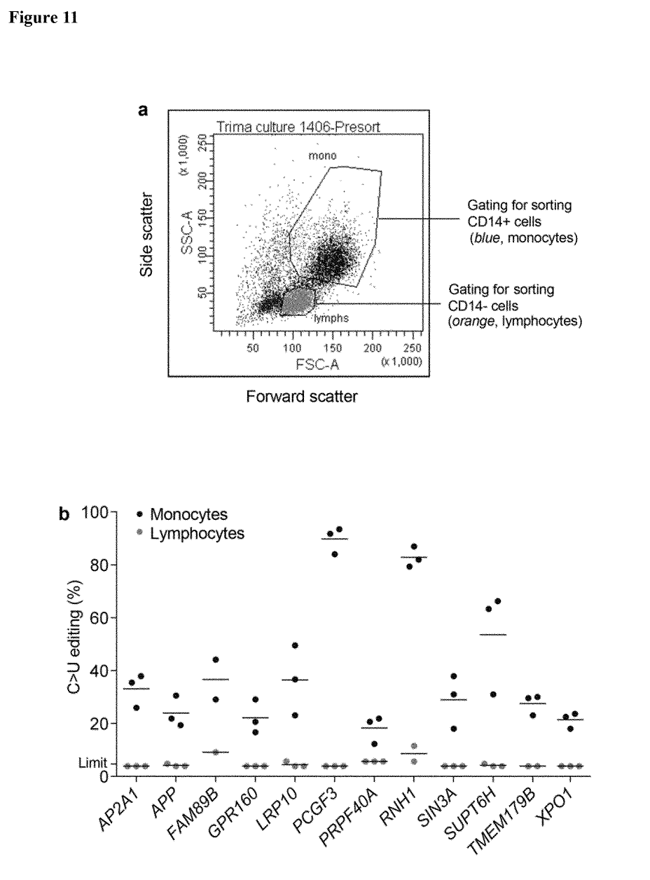

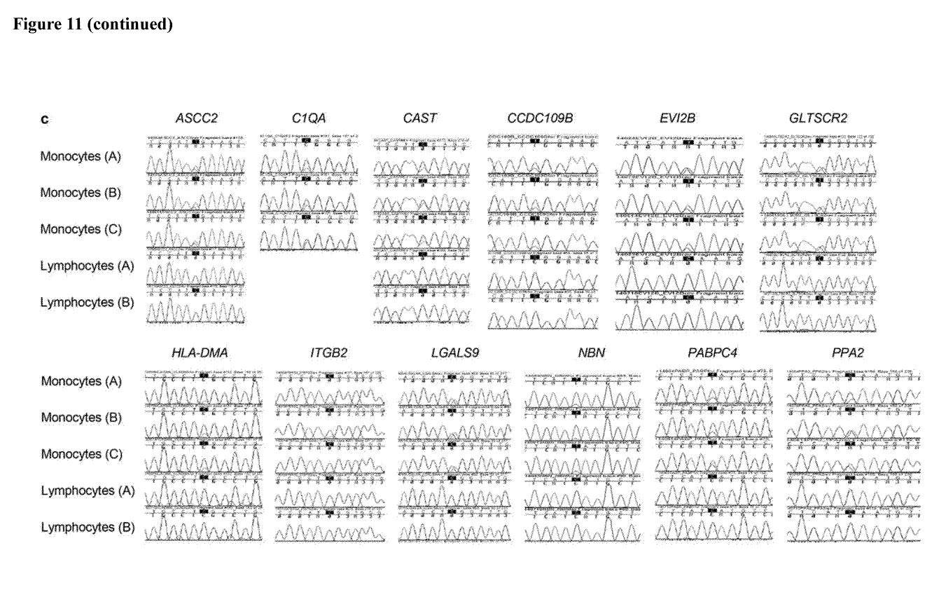

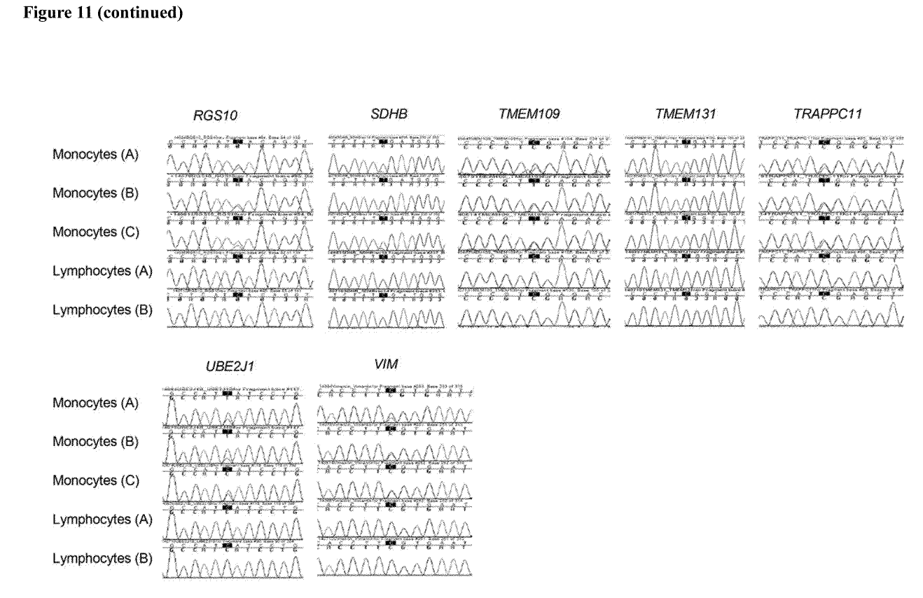

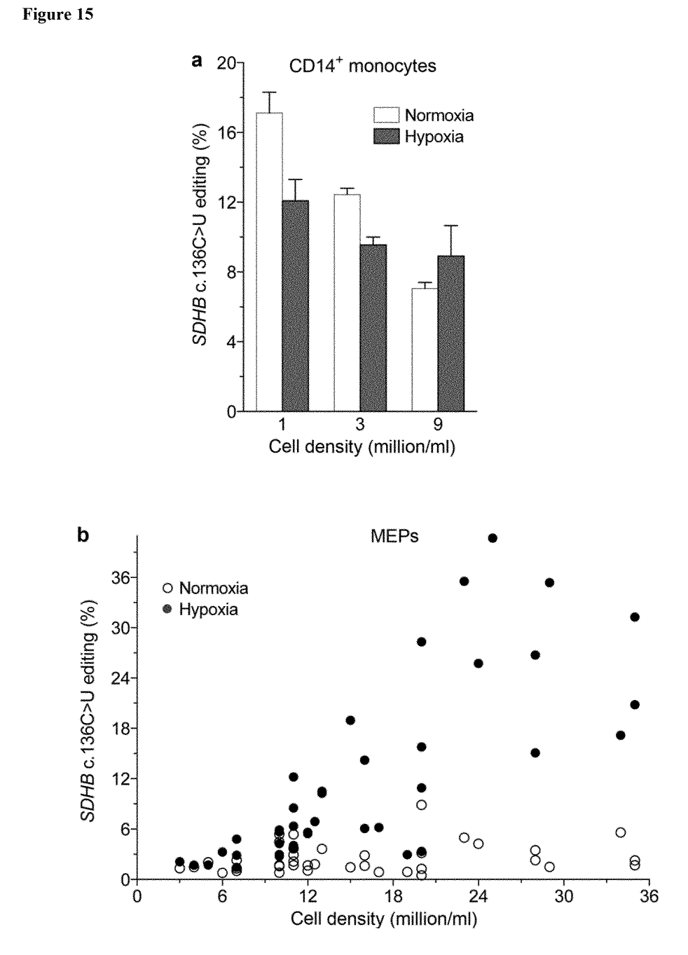

The TLA-HEK293T™ 293T human embryonic kidney cell-line was obtained from Open Biosystems® (Huntsville, Ala.). Peripheral blood mononuclear cells of anonymous platelet donors were isolated from peripheral blood in Trima Accel™ leukoreduction system chambers (Terumo BCT®, Lakewood, Colo.) after thrombocytapheresis, in accordance with a protocol approved by the institutional review board of Roswell Park Cancer Institute. A density gradient centrifugation method using polysucrose-containing Lymphocyte Separation Medium (Mediatech®, Manassas, Va.) was used for PBMC isolation. MEPs were prepared from PBMCs using the well-established cold aggregation method (Mentzer et a., Cell Immunol 101, 312-319 (1986) with slight modification. Briefly, PBMCs were subjected to gentle rocking at 4° C. for an hour and aggregated cells that sedimented through fetal bovine serum (FBS; VWR®, Radnor, Pa.) were collected as MEPs after 0.5-3 hours for high monocyte enrichment (˜70% monocytes as assessed by immunofluorocytometry for CD14), or after 8-16 hours for mild enrichment (˜20%-40% monocytes); the latter was used in all experiments except for the ones of FIG. 1a. MEPs were cultured at a density of 13-63 million per ml (mean=29 million per ml, n=16 experiments) in 1 or 2 ml per well of 6- or 12-well standard tissue culture plates under 5% CO2 in RPMI-1640 medium (Mediatech®) with 10% FBS, and 100 U per ml penicillin and 100 μg per ml streptomycin (Mediatech®). Monocytes and lymphocytes were isolated from MEPs based on light scattering and binding of a phycoerythrin-conjugated mouse anti-CD14 antibody (clone RM052, product number 6699509D, 1:40 dilution, Beckman Coulter®, Miami, Fla.) by flow cytometry on a FACS Aria™ II instrument with FACS Diva™ 6.0 software (BD Biosciences®, San Jose, Calif.) (FIG. 11a). CD14+ monocytes used in the experiment for FIG. 1b were isolated from PBMCs using mouse anti-CD14 antibody-conjugated microbeads and magnetic separation on an AutoMACS™ instrument (Miltenyi Biotec®. Auburn, Calif.). Monocytes used in the experiment for FIG. 3c were isolated from MEPs by immunomagnetic negative selection using EasySep™ Human Monocyte Enrichment Kit (Stemcell Technologies®, Vancouver, Canada). For the APOBEC3A knock-down experiment, monocytes of 70% CD14 positivity were isolated from PBMCs using a centrifugation-based method (Seager Danciger et al., J Immunol Methods 288, 123-134 (2004)) with a single-layer of iso-osmolar, 42.5% v/v solution of Percoll™ (GE Healthcare®, Pittsburgh, Pa.) in RPMI-1640 medium with 10% FBS. Except for the cells used in the experiment for FIG. 1b, all primary cells were used in experiments immediately after their isolation from PBMCs. Enhancement by hypoxia of SDHB c.136C>U RNA editing was not observed in cultures of previously cryopreserved CD14+ monocytes (FIG. 15a). Hypoxic induction of RNA editing was also not consistently observed in hypoxia if freshly isolated MEPs were cultured at a low cell density (<10 million cells per ml (FIG. 15b).

### Generation and Polarization of Macrophages

CD14+ monocytes isolated from PBMCs by magnetic sorting and stored frozen in RPMI-1640 with 36% v/v FBS and 10% v/v dimethyl sulfoxide, were thawed and cultured for a week at a density of 0.25 million per ml with 50 ng per ml recombinant human macrophage colony stimulating factor (MCSF; Life Technologies®, Carlsbad, Calif.), 1× GlutaMAX™-I (Life Technologies®) and 1 mM sodium pyruvate (Mediatech®) to generate M0 macrophages. M0 macrophages were also similarly generated from fresh monocytes isolated from PBMCs by the Percoll™-based method. For M1 or M2 macrophage polarization, M0 cells were treated for two days with 20 ng per ml recombinant human IFNγ (Life Technologies®) and 100 ng per ml E. coli lipopolysaccharides (LPS; List Biological Laboratories®, Campbell, Calif.), or 20 ng per ml recombinant human interleukin 4 (Life Technologies®), respectively. RNA was isolated from cells using the Total RNA Purification Kit from Norgen Biotek® (Thorold, Canada).

### Hypoxia and Interferon Treatments

For hypoxia, cells were cultured under 1% 02, 5% CO2 and 94% N2 in an Xvivo™ System (Biospherix®, Lacona, N.Y.). Human IFNγ and ‘universal’ type I IFN, a hybrid of N-terminal IFNα-2 and C-terminal IFNα-1, produced in E. coli were obtained from PBL Assay Science® (Piscataway, N.J.), and respectively used at 200 and 300-1,500 U per nil. Unless noted otherwise, hypoxia and/or interferon treatments were for 24 hours. Differential viability of MEPs after 1-day culture in normoxia versus hypoxia was not observed as evaluated by Trypan blue stain. Transfected 293T cells were subjected to hypoxia and/or interferon treatment 24 hours after transfection.

### RNA Sequencing of MEPs

Indexed sequencing libraries were generated from RNA, isolated using TRIzol™ and without DNAse treatment, as per methods and reagents provided with the TruSeq™ Stranded Total RNA Sample Prep Kit with Ribo-Zero™ ribosomal RNA reduction chemistry (Illumina®, San Diego, Calif.). PCR for library generation employed 10 cycles. Electrophoresis of the libraries on Bioanalyzer™ 2100 instrument (Agilent®, Santa Clara, Calif.) showed highest peaks at 220-240 bp. Paired-end, multiplexed sequencing of libraries (three per flow cell lane) to generate reads of 101 bases (b) was performed on HiSeg™ 2000 instrument with TruSeq™ SBS and PE Cluster v3 Kits (Illumina®). CASAVA 1.8.2 software (Illumina®) was used for base-calling and de-multiplexing to obtain the raw RNA sequencing reads for further analyses. RNA sequencing of all six samples of this study was performed in one batch.

### Macrophage RNA Sequencing Data of Beyer et al.

Paired-end, 101 b read sequence data generated using TruSeq™ RNA Sample Preparation Kit on Illumina® HiSeq™ 2000 for paired M1 and M2 macrophages derived from CD14+ monocytes of three donors was obtained as SRA files from NCBI SRA (accession number SRP012015). Raw data in fastq format was extracted from the files with fastq-dump utility in NCBI SRA Toolkit 2.3.3 (ncbi.nlm.nih.gov/sites/books/NBK158900/).

### Processing of RNA Sequencing Reads

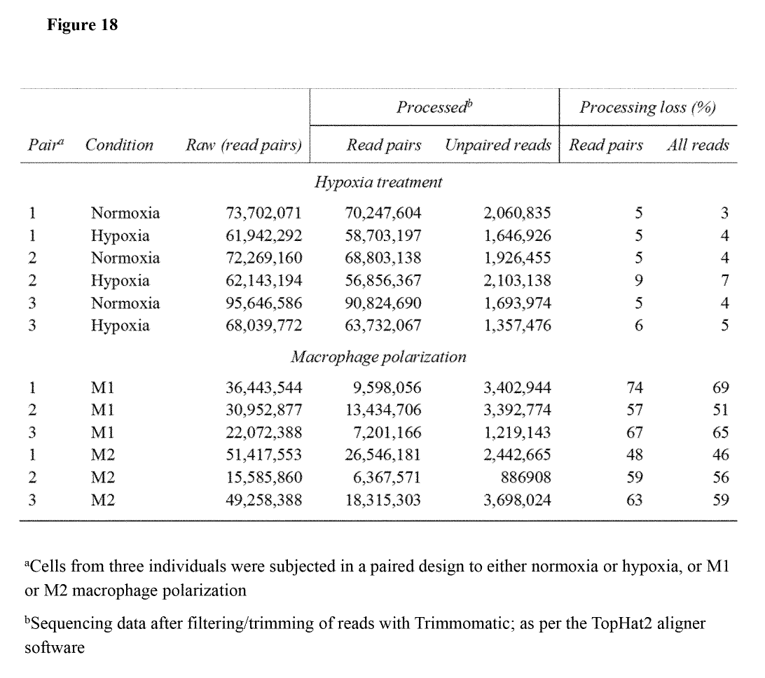

Quality of reads was assessed using FastQC 0.10.1 (URL: www.bioinformatics.babraham.ac.uk/projects/fastqc/). Trimmomatic 0.32 (URL: www.usadellab.org/cms/?page=trimmomatic) was used to trim 12 b from 5′ end, and remove adapter sequences and poor-quality bases from the reads. The Trimmomatic call was invoked with ‘HEADCROP:12 ILLUMINACLIP: TruSeq3-PE-2.fa:2:30:10:6:TRUE LEADING:5 TRAILING:5 SLIDINGWINDOW:4:15 MINLEN:30’, to satisfy these criteria, in order: (1) remove 12 b from the 5′ end of all reads because of base bias at these positions; (2) remove read segments that matched sequences of adapters and primers used for sequencing library preparation (the TruSeq3-PE-2.fa file provided with Trimmomatic was used); (3) remove leading/trailing bases with Phred33 base quality score <5; (4) using a sliding window of four bases, remove the most 5′ base if the average Phred33 base quality score of the four bases was <15; and, (5) completely discard trimmed reads with <30 remaining bases. Pair-mates of a fraction of raw reads were lost following this read processing with Trimmomatic. Processed read data thus had both paired and unpaired reads (FIG. 18).

### Mapping of Processed RNA Sequencing Read Pairs

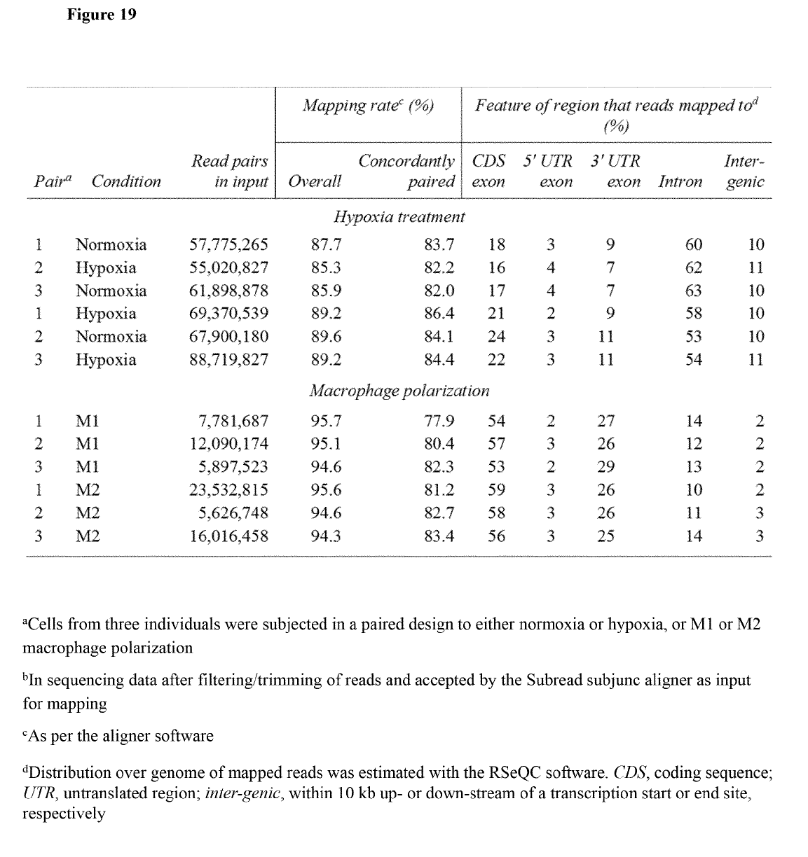

Processed read pairs were uniquely mapped to the hg19 genome with the Subread (Liao et al., Nucleic Acids Res 41, e108 (2013)) subjunc 1.4.3-pl aligner. The subread-buildindex command of Subread was used with default argument values to index the whole genome FASTA file for the UCSC hg19 genome assembly (obtained from Illumina® iGenomes). Subread subjunc command was used for mapping paired reads to the genome using the genome index with arguments u and H but otherwise default argument values to permit only unique mapping of a read and using Hamming distance to break ties when there were more than one best mappings. The nature of genomic regions that the reads mapped to was assessed using RSeQC 2.3.7. Mapping statistics are provided in FIG. 19. To obtain gene-level mapped read count data, the mapping results (BAM files) were analyzed with Subread featureCounts with reference to the GTF gene annotation file from UCSC (6 Mar. 2013 version) in the Illumina® iGenomes UCSC hg19 data with the following argument values specified: isStrandSpecific (s)=2 (0 in case of the macrophage data), GTF.featureType (t)=exon, GTF.attrType (g)=gene_id, isPairedEnd (p), allowMultiOverlap (0).

Processed RNA Sequencing Reads Mapping with TopHat2 Aligner

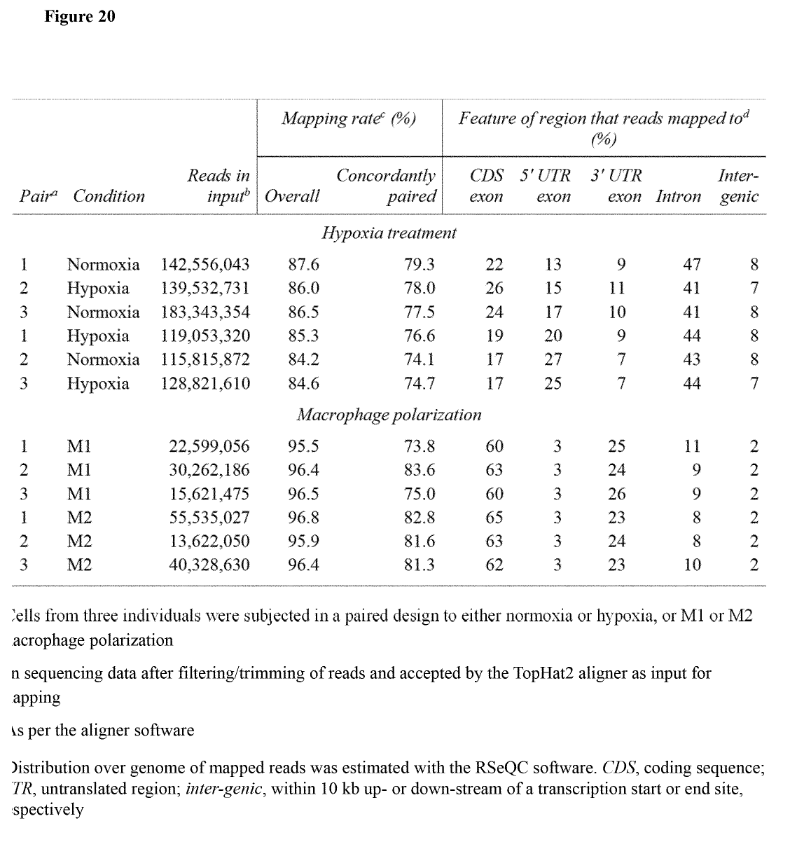

Processed reads, both paired and unpaired, were also mapped to the UCSC hg19 human genome assembly with the TopHat2 2.0.10 aligner, permitting only unique mapping of a read with up to three nucleotide mismatches. The bowtie2 index for the UCSC hg19 genome assembly was obtained from ftp.ccb.jhu.edu/pub/data/bowtie2_indexes, and the transcriptome index was built with TopHat2 using the GTF gene annotation file from UCSC (6 Mar. 2013 version) in the Illumina® iGenomes UCSC hg19 data. The tophat2 command was used with the transcriptome and bowtie2 genome indexes, and the following argument values specified: mate-inner-dist=−50, mate-std-dev=40, max-multihits=1, read-mismatches=3, read-edit-dist=3, no-novel-juncs (and library-type=fr-firststrand, in case of the RNA sequencing data of MEPs). The nature of genomic regions that the reads mapped to was assessed using RSeQC (URL: rseqc.sourceforge.net). Mapping statistics are provided in FIG. 20.

### Generation of Mapped RNA Sequencing Read Pileups

After clipping overlaps of read pair-mates with clipOverlap utility in bamUtil 1.0.10 (URL: genome.sph.umich.edu/wiki/BamUtil), pileups were produced from the mapping data (BAM files) with mpileup in SAMtools 0.1.19 (URL: www.htslib.org), with computation of base alignment quality disabled (B), ‘anomalous’ reads permitted (A), maximum depth (d) set at 80,000, and aligner-reported read mapping quality (Q) >0 and Phred33 base quality score (q) >19 required.

### Analyses of Mapped RNA Sequencing Read Pileups

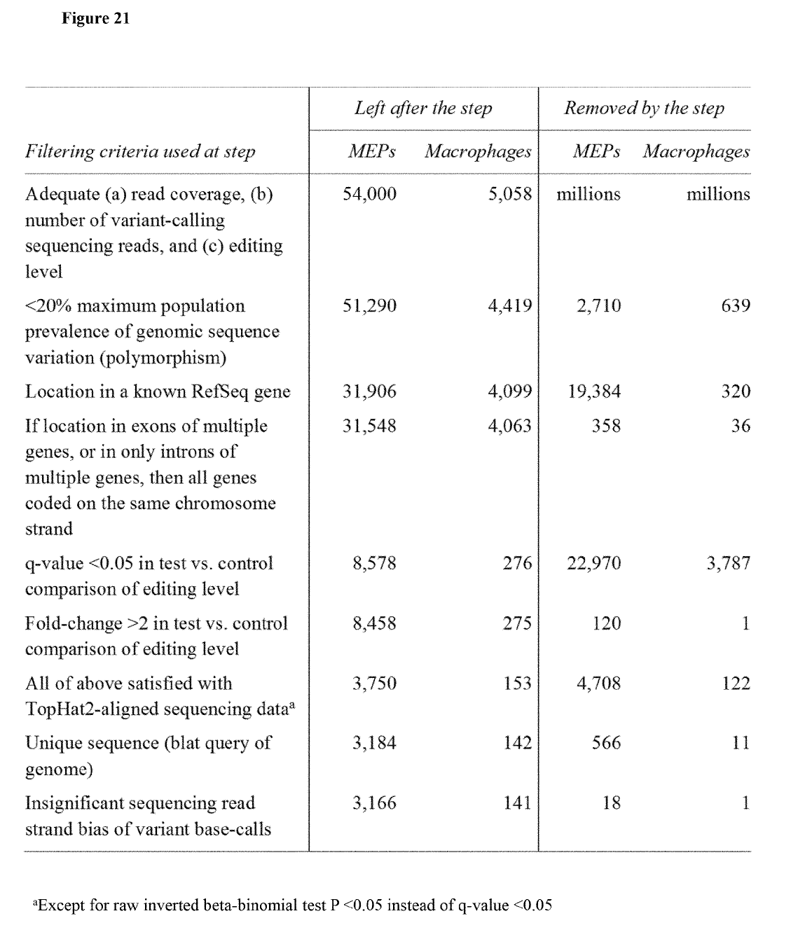

Paired comparison of pileups for the three pairs, from three different human donors, of normoxic and hypoxic MEP, or M2 and M1 macrophage samples was performed to identify genome sites with differential RNA editing in MEPs under hypoxia (test samples) compared to normoxia (control samples), or in M1 macrophages (test) compared to M2 macrophages (control). Python 2.7, R 3.0 and shell scripts were used for the analysis. Sites considered for analysis satisfied all of the following criteria regarding the A/T/G/C base-calling reads that covered them: (1) ≧20 calls (per sample, as for the other criteria here) in both samples of ≧1 pair, and ≧5 calls in all six samples; (2) ≧50% of calls for the reference human genome base in all test or all control samples; (3) ≧2 variant (other than the reference base) but identical base-calls in ≧2 test or ≧2 control samples, with ≧1 such calls in all test or all control samples, and ≦5 base-calls for a different variant in all six samples; and, (4) ≧95% reads with a base-call for either the reference or variant nucleotide in all six samples (thus, only one type of nucleotide change was considered for a site). Variation or editing level for sites was calculated as the ratio of variant base-calling- to the sum of variant and reference base-calling-read counts. Sites were then filtered by editing level, requiring: (1) ≧2.5% in ≧2 test or ≧2 control samples; (2) mean≧5% for test or control samples; and, (3) range/mean≧2 across all six samples (to reduce subsequent multiple testing). Variant sites with known maximum population prevalence >20% for identical sequence polymorphism (as per the popfreq_max ANNOVAR database, detailed below), or sites that did not map to a known RefSeq gene (URL: www.ncbi.nlm.nih.gov/refseq), or mapped to either exons of >1 RefSeq genes on both chromosome strands, or mapped to only introns of >1 RefSeq genes on both chromosome strands were excluded. Annotation data (BED files) for RefSeq gene introns and exons for the UCSC hg19 genome assembly were obtained on 21 Mar. 2014 using UCSC Genome Browser (URL: genome.ucsc.edu). The inverted beta-binomial (IBB) test for multiple paired count data was then applied to the remaining variant sites to identify sites that were differentially edited between the test and control samples. To control false discovery resulting from multiple testing, q-values were calculated from IBB test P values using the qvalue function in the qvalue Bioconductor package with these argument values specified: pi0.method=bootstrap, robust. Sites that were further considered had q-value<0.05 and >2-fold difference in either direction for editing level between test and control samples (fold-change values, capped at an absolute value of 104, were estimated by the IBB test) in analysis of Subread subjunc-aligned RNA sequencing data as well as an IBB test P value<0.05 and >2-fold difference in analysis of TopHat2-aligned RNA sequencing data. Sites were then filtered if either of their 5′ and 3′, 29 b-long, flanking genomic sequences, respectively with either the reference or variant base at the 3′ and 5′ end, aligned perfectly with the genome at another location; blat 35 (URL: genome.ucsc.edu) was used for this purpose. Finally, for filtering based on sequencing read strand bias, sites were filtered out if in the Subread subjunc-aligned data the variant base was called from a total across all six samples of >9 forward RNA sequencing reads but no reverse read, or vice versa, or if the number of forward and reverse reads were significantly different for either the test or control samples (IBB test P value<0.05). Numbers of sites that were left after and filtered by different steps of the analysis described here are noted in FIG. 21. RNA-level nucleotide change was deduced from DNA alteration based on the chromosome strand coding for the gene that a site mapped to, using the exon-bearing strand if a site mapped to both an intron and exon on opposite strands.

### Analyses of RNA Editing Sites

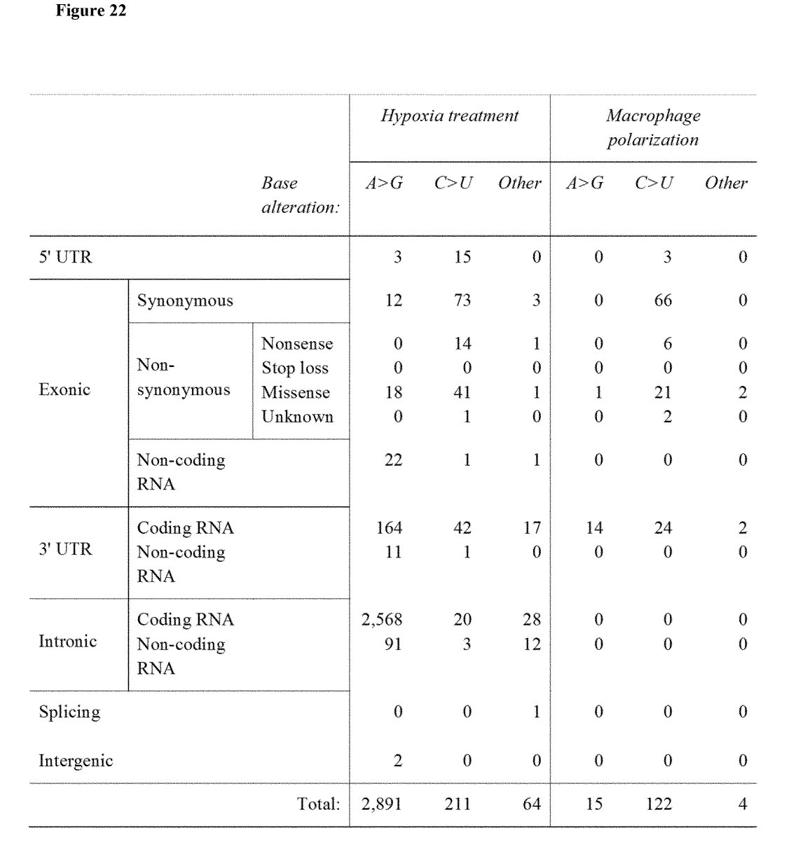

ANNOVAR tool (23 Aug. 2013 release; openbioinformatics.org/annovar) and 1jb23_metalr (22 Feb. 2014), popfreq_max (21 Aug. 2013), RefSeq-based refGene (13 Nov. 2013), and dbSNP 138-based snp138 (13 Dec. 2013) ANNOVAR databases were used to annotate sites with information such as gene features they are located in, frequencies of known C/T genomic DNA polymorphism, and effects on amino acid coding. Coding genomic strand sequences flanking the editing sites were extracted from the whole genome FASTA file for the UCSC hg19 genome assembly (obtained from Illumina® iGenomes) with the getfasta utility in bedtools 2.17.0 (github.com/arq5x/bedtools), and these sequences were analyzed as transcript RNA sequences. Palindromic sequence context of editing sites was manually examined. RNA folding was predicted with ViennaRNA package 2.1.6 (tbi.univie.ac.at/RNA). These annotations are provided in Supplementary Data 1 in FIG. 22. Annotations on gene feature and amino acid coding change are summarized in Supplementary table 5. WebLogo 3 online tool was used to create sequence logos (weblogo.threeplusone.com). Gene set enrichment analyses for biological function, molecular process and PANTHER pathway ontologies were performed with PANTHER 9.0 (pantherdb.org/panther). Enrichment of a gene set with at least two genes for an ontology term, in comparison to the reference database for 21,804 genes, was assessed by binomial test and an FDR<5%, calculated from P values by the Benjamini-Hochberg method, was considered significant.

### Gene Expression, RNA and Whole Exome Sequencing Data

Level 3, gene-level expression data determined by RNA sequencing with the UNC v2 pipeline were obtained from Broad Institute GDAC Firehose (2014_03_16 stddata run). RNA and whole sequencing data mapped to the hg19 genome assembly (BAM files) were obtained from Cancer Genomics Hub (University of California, Santa Cruz) respectively during February and March, and October 2014.

### Analysis of TCGA Tumor RNA Sequencing Data for SDHB Editing

Pileups were generated as described above. Editing was deemed indeterminable for a sample if <99% of mapped reads had a base-call other than A or G, or there were <200 calls with none for A, or there were <100 calls with only one for A (SDHB gene is coded on the minus chromosome strand). Otherwise, C>U editing level was estimated as the ratio of G to the sum of A and G calls. Information on C/T single nucleotide polymorphisms in SDHB protein coding sequence was obtained from dbSNP (build 37).

### RNA Sequencing Data Analysis for Varied Gene Expression

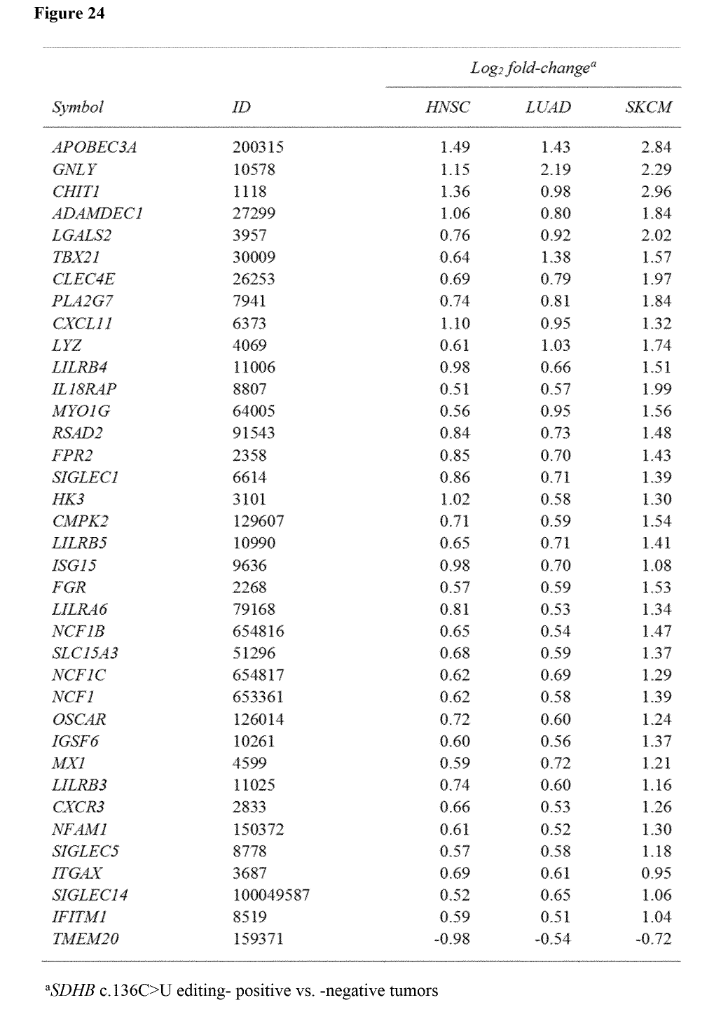

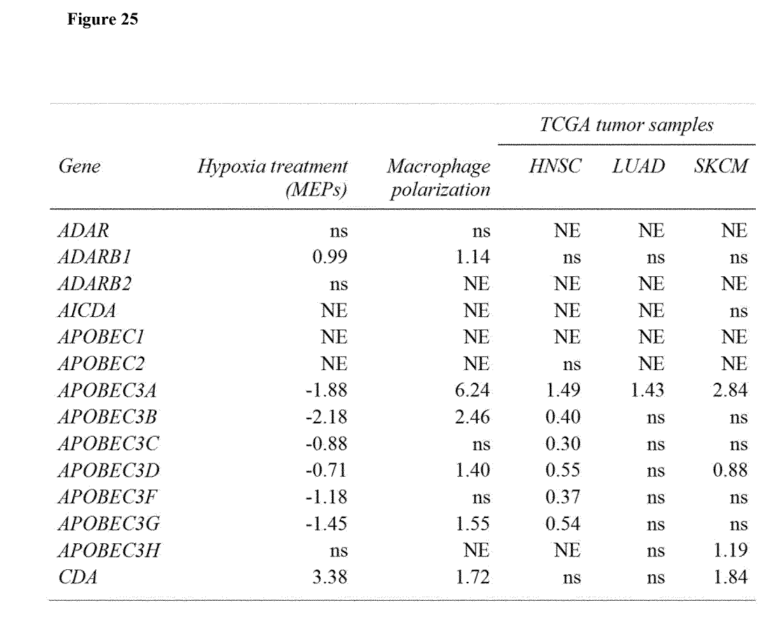

Gene-level raw count values of transcripts were analyzed with the edgeR Bioconductor package (version 3.2.3) for normalization with the trimmed mean of M-values method and inter-group comparison of gene expression by exact or likelihood ratio tests. For analyses of RNA sequencing data of tumor samples of TCGA, genes with raw count value>0 for ≧N samples, irrespective of group membership, where N equals the size of the SDHB c.136C>U editing-positive group, were considered as expressed, and values for prior.df and rowsum.filter parameters in estimateCommonDisp and estimateTagwiseDisp functions of edgeR were respectively set at 0.2 and 4N. An exact test was used to generate P values. For analyses of RNA sequencing data of MEPs and macrophages, genes with raw count value>1 for ≧3 samples, irrespective of group membership, were considered as expressed, and pair-wise comparison of gene expression between groups using generalized linear models with negative binomial distribution and a likelihood ratio test to generate P values was performed. False discovery rates (FDR) were estimated from P values with the Benjamini-Hochberg method, and genes with FDR<0.05 were considered as differentially expressed. Summarized results of differential gene expression analyses are provided in FIGS. 24 and 25.

### Gene Expression Constructs and Site-Directed Mutagenesis

Sequence-verified plasmid constructs in pCMV6 vector for CMV promoter-driven expression of human APOBEC3A, APOBEC3G, CDA and SDHB cDNAs, with sequences matching NCBI RefSeq sequences NM_145699.2, NM_021822.1, NM_001785.1 and NM_003000.2, respectively, for the generation of C-terminal Myc-DDK-tagged APOBEC3A, and untagged APOBEC3G, CDA and SDHB proteins were obtained from OriGene® (Rockville, Md.; product numbers RC220995, SC122916, SC119015 and SC319204, respectively). An inducible bacterial expression construct for APOBEC3A with a C-terminal His6-tag in the pET21 vector was obtained from Dr. Jinwoo Ahn (University of Pittsburgh, USA). Site-directed mutagenesis of APOBEC3A constructs (c.216G>C/p.E72D, c.301T>A/p.C101S or c.400C>G/p.P134A; primer sequences shown in FIG. 27) was performed using QS™ mutagenesis kit (New England Biolabs®, Ipswich, Mass.). Sequences of cDNA inserts of all of these constructs except that for SDHB were verified by Sanger sequencing. Insert-less pcDNA™ 3.1(+) vector (Life Technologies®) plasmid was used for control transfectants. The pRL-SV40 plasmid for SV40 promoter-driven expression of Renilla luciferase was obtained from Addgene® (Cambridge, Mass.). A LINE-1 plasmid (Mitra et al. Nucleic Acids Res 42, 1095-1110 (2014)) with an ˜6 kb human LINE-1 element bearing a CMV promoter-driven firefly luciferase cassette in its 3′ untranslated region was obtained from Dr. Judith Levin (National Institute of Child Health and Human Development, USA).

### Transfection of Plasmid DNA

293T cells were transfected with plasmid DNA using the liposomal X-tremeGENE™ 9 DNA reagent (Roche®, Indianapolis, Ind.) or jetPRIME™ (Polyplus-Transfection®, New York, N.Y.) reagents as per guidelines provided by the reagent manufacturer. Transfection efficiency with both reagents was 60%-80% as assessed by fluorescent microscopy of cells transfected with the pLemiR™ plasmid DNA (Open Biosystems®) for expression of a red fluorescent protein. Cells were harvested two days after transfection.

### Knock-Down of APOBEC3A RNA in M1 Macrophages

A day before induction of M1 polarization, M0 macrophages at a density of 1 million cells per ml in 1 ml medium per well of 6-well plates were transfected with 100 nM of negative control (Silencer™ negative control no. 1, product number AM4611, Life Technologies®) or either or equimolar mix of two human APOBEC3A siRNAs (Silencer™ 45715 and 45810 respectively with sense sequences GACCUACCUGUGCUACGAATT (SEQ ID NO:1) and GCAGUAUGCUCCCGAUCAATT (SEQ ID NO:2), Life Technologies®) using Lipofectamine RNAiMAX™ (Life Technologies®) as per guidelines supplied by the manufacturer. IFNγ and LPS were added with 1 ml medium to each well to induce M1 polarization, and cells were harvested a day later.

### LINE-I Retrotransposition Assay

Briefly, firefly luciferase expression conditional to the retrotransposition of a human LINE-1 element from a plasmid DNA to the genome is measured in this assay. 293T cells at ˜50% confluence in 12-well tissue culture plates were co-transfected with 0.75 μg of the LINE-1 plasmid, 0.5 μg of pcDNA™ 3.1(+) or an APOBEC3A expression plasmid, 0.25 μg of pcDNA™ 3.1(+), and 1 ng of pRL-SV40 plasmid (per well). Transfectants were lysed after two days for measurement of their firefly and Renilla luciferase activities using Dual-Luciferase™ Reporter Assay System (Promega®). Retrotransposition was quantified as the ratio of firefly and Renilla luciferase activities.

### Reverse Transcription and PCR

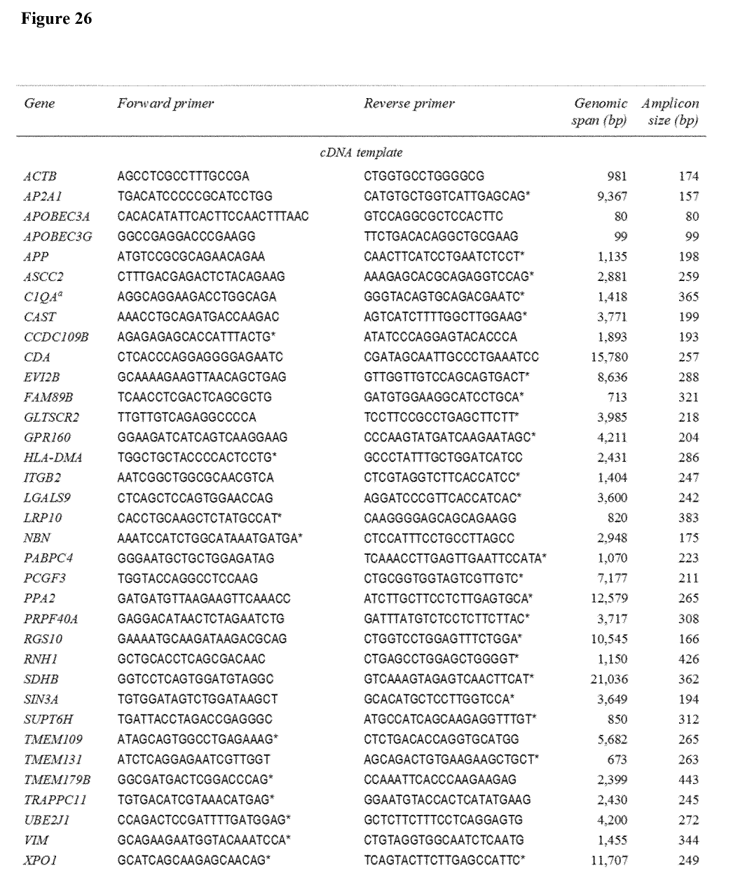

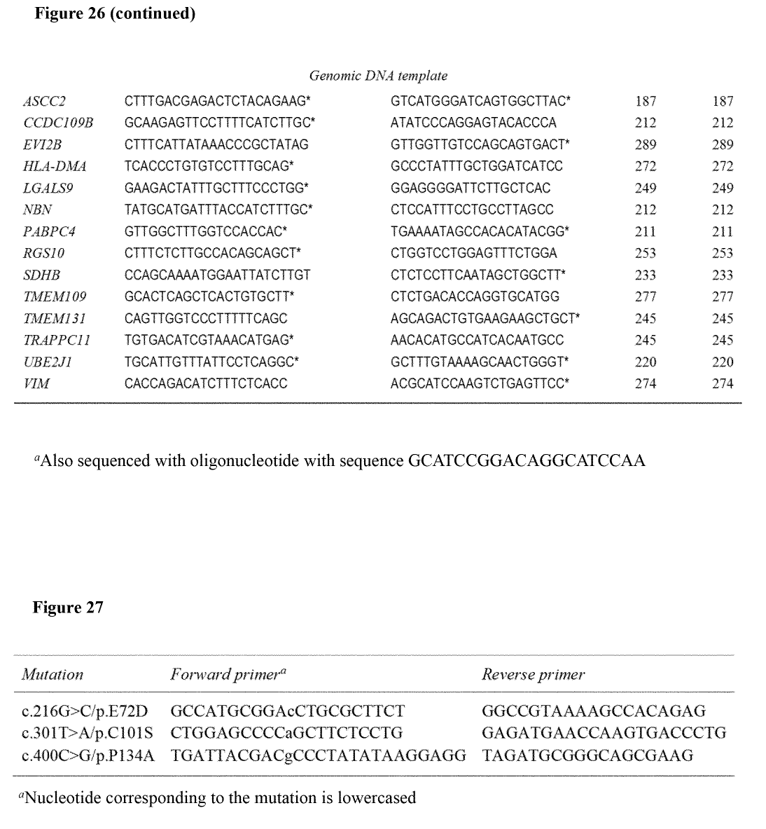

RNA was reverse transcribed with random DNA hexamers and/or oligo-dT primers using material and methods provided with the Transcriptor™ First Strand cDNA Synthesis (Roche®) or High Capacity cDNA Reverse Transcription (Life Technologies®) kits. PCR typically employed 35 cycles of amplification and an annealing temperature of 60° C. PCR oligonucleotide primers (Integrated DNA Technologies®, Coralville, Iowa) are listed in FIG. 26. Electrophoresis of PCR reactions on agarose gel was used to confirm the generation of a single product in a PCR. Primers used for PCR of cDNA templates were designed such that the amplicons spanned multiple exons. A blend of Taq and high-fidelity Deep VentR™ DNA polymerases (OneTaq™, New England Biolabs, Ipswich, Mass.) was used in PCR to generate products for Sanger sequencing. For quantitative PCR to assess ACTB, APOBEC3A, APOBEC3B, CDA, SDHB, SIN3A and B2M gene expression, reactions using FastStart™ Taq DNA polymerase and SYBR™ Green I dye were performed on a LightCycler™ 480 System (Roche®). Quantification cycle (Cq) values were calculated by the instrument software using the maximum second derivative method, and the mean Cq value of duplicate or triplicate PCR reactions was used for analysis. TaqMan™ Gene Expression Assays from Life Technologies® with identification numbers Hs00234140_ml, Hs00171149_ml, Hs00233627_ml and Hs00267207_ml, or prepared in house were respectively used to quantify CCL2, CCL19, FCER2, MRC1 and ACTB with PCR performed on a 7900HT instrument (Life Technologies®) and Cq values determined with automatic baseline and threshold detection by SDS 2.4 software (Life Technologies®).

### Sanger Sequencing

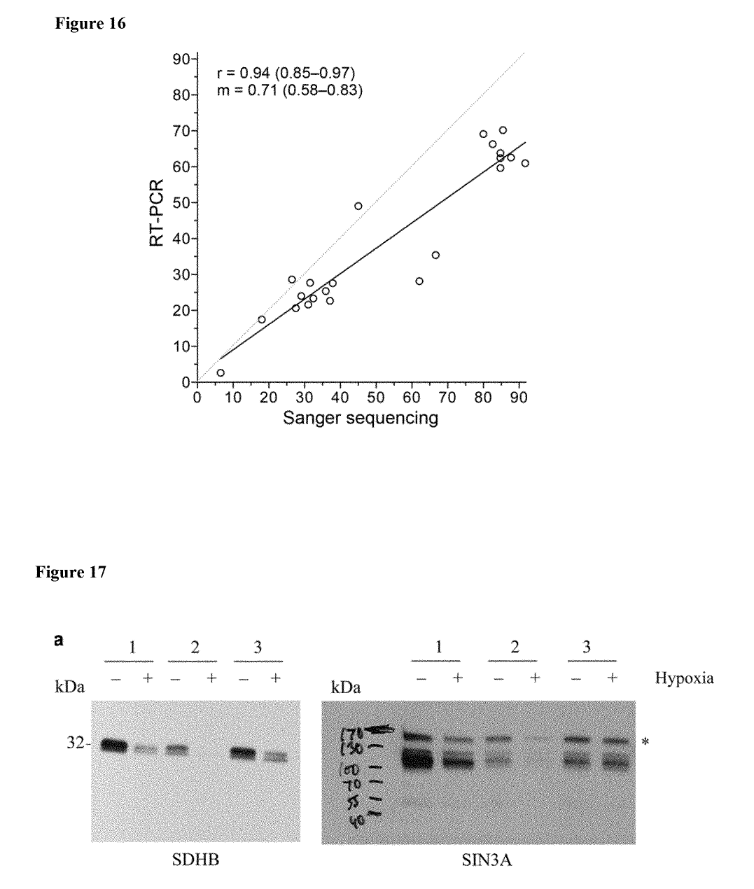

Sequencing primers (Integrated DNA Technologies®) are listed in FIG. 26. Candidate C>U RNA editing sites for which PCR-amplified genomic DNA and cDNA fragments were sequenced are noted in Table 1. PCR reactions were treated with ExoSAP-IT™ exonuclease (Affymetrix®, Santa Clara, Calif.) and then directly used for sequencing on 3130 xL Genetic Analyzer™ (Life Technologies®). Major and minor chromatogram peak heights at a nucleotide position of interest were quantified with Sequencher™ 5.0 software (Gene Codes®, Ann Arbor, Mich.) to calculate editing level for the position. Because the software identifies a minor peak only if its height is >5% of the major peak's, a relative minor peak height value of 4% was assumed to assign an editing level of 3.8% when a minor peak was absent. Appropriateness of this method to estimate RNA editing level was confirmed by comparing measurements of SDHB c.136C>U RNA editing level obtained with it against those obtained with allele-specific RT-PCR (FIG. 16).

### Immunoblotting of Cell Lysates

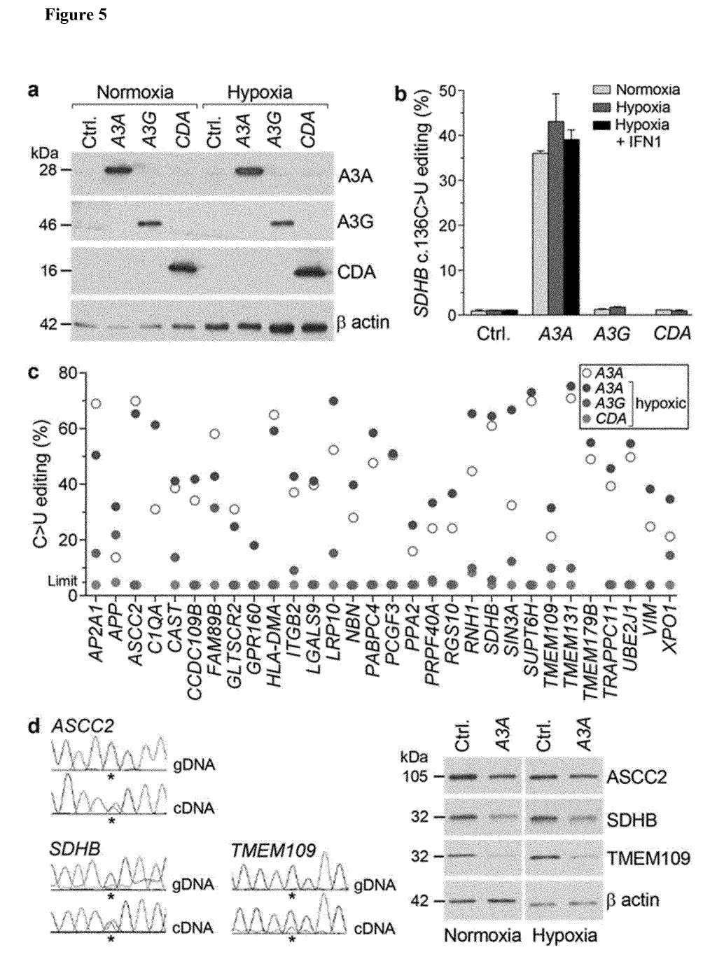

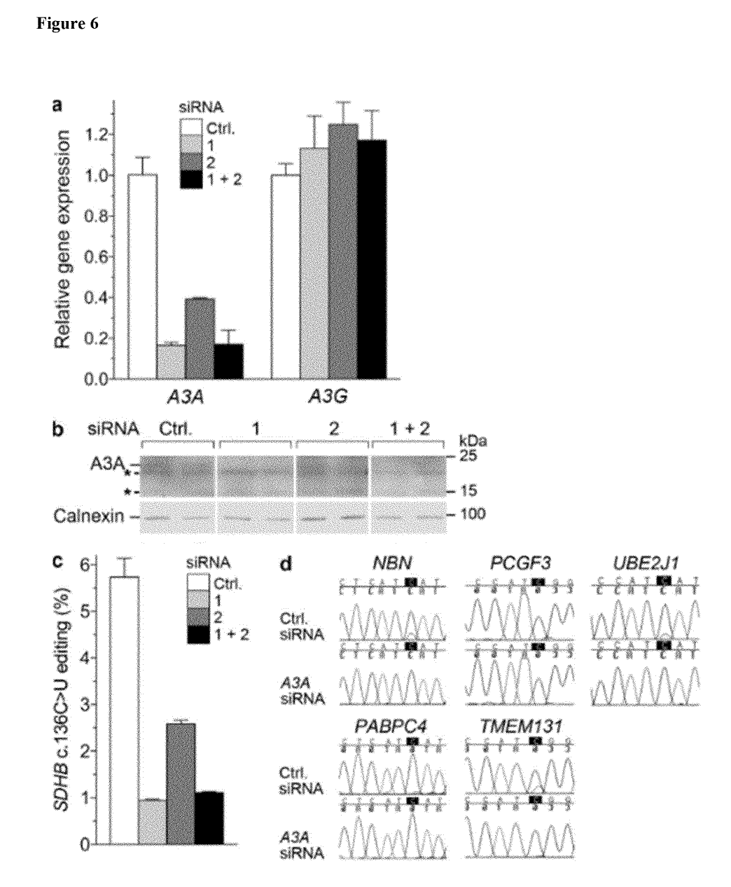

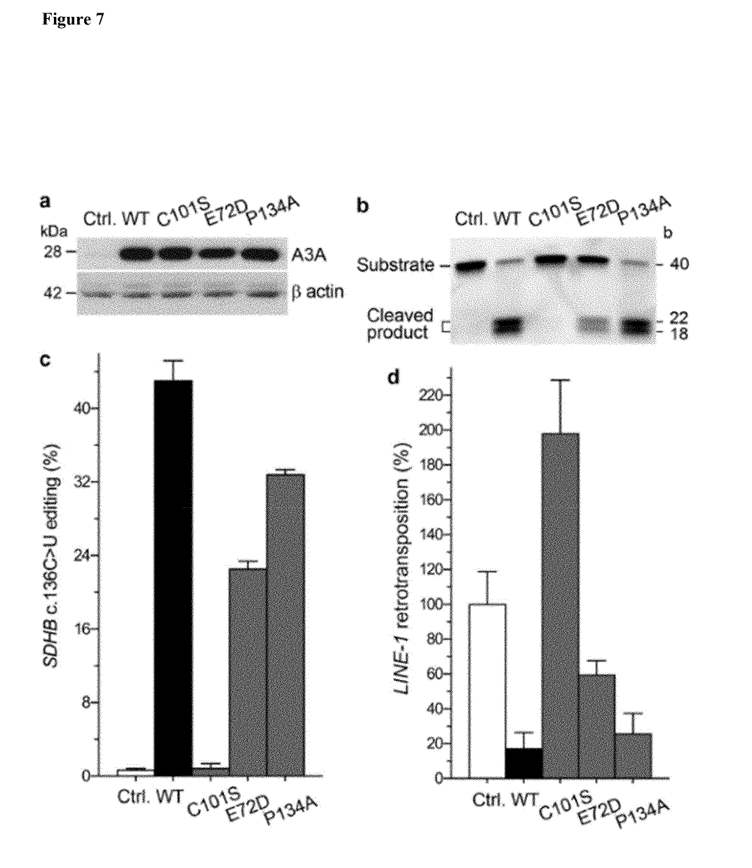

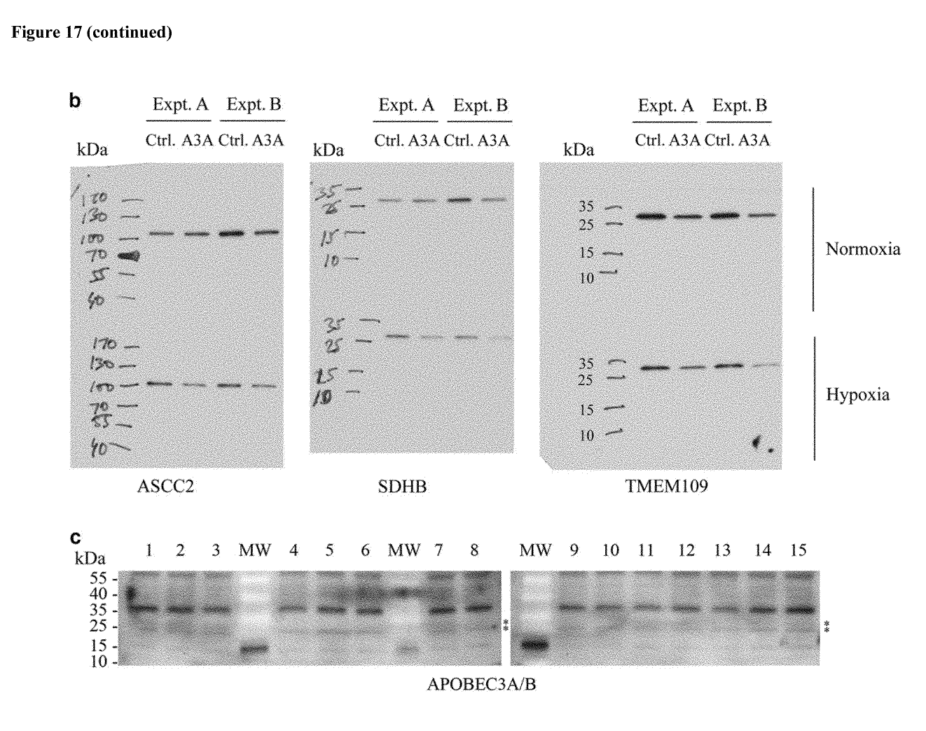

Whole cell lysates were prepared using M-PER™ reagent (Thermo Fisher®, Rockford, Ill.) with 1× Halt™ protease and phosphatase inhibitor cocktail (Thermo Fisher®). Reducing and denaturing polyacrylamide gel electrophoresis of 20 μg proteins in Laemmli buffer system was performed on pre-cast, 4%-15% gradient polyacrylamide gels (Mini-PROTEAN TGX™, Bio-Rad®, Hercules, Calif.). Proteins were then transferred to polyvinylidene difluoride membrane with a pore-size of 0.2 μm for 7 minutes at 1.3 A in a Bio-Rad® Trans-Blot Turbo™ apparatus. Membranes were incubated in Tris-buffered 0.15 M NaCl of pH 7.5 with 0.05% v/v TWEEN™ 20 (Sigma Aldrich®, Saint Louis, Mo.) and 5% w/v dried, non-fat, cow milk (Carnation™, Nestlé®, Glendale, Calif.) with antibodies at dilutions recommended by their manufacturers. Rabbit polyclonal anti-APOBEC3A (product number sc-130688, D-23, 1:200 dilution; used in the experiments for FIGS. 5a and 7a), anti-APOBEC3A/B (product number sc-292434, H-89; used in the experiment for FIG. 6b, 1:150 dilution), anti-ASCC2 (product number sc-86303, T-16; raised against peptide from internal region of human ASCC2, 1:200 dilution), and anti-TMEM109 (product number sc-133788, D-23; raised against human TMEM109 peptide of undisclosed sequence, 1:200 dilution) antibodies, and mouse monoclonal anti-CDA (product number sc-365292, D-5, 1:500 dilution) and anti-SDHB (product number sc-271548, G-10; raised against human protein of full length, 1:500 dilution) antibodies were obtained from Santa Cruz Biotechnology® (Santa Cruz, Calif.). Rabbit polyclonal anti-APOBEC3G (product number ab38604, 1:8000 dilution), mouse monoclonal anti-β-actin (product number AM4302, 1:15,000 dilution), and rabbit monoclonal anti-SIN3A (product number MABE607, EPR6780; raised against peptide near C-terminus of human SIN3A, 1:3000 dilution) antibodies were respectively obtained from Abcam® (Cambridge, Mass.), Life Technologies®, and EMD Millipore® (Billerica, Mass.). Rabbit polyclonal anti-calnexin antibodies (product number GTX10966, C3, 1:2000 dilution) were purchased from GeneTex (Irvine, Calif.). Horse radish peroxidase-conjugated, goat anti-mouse or -rabbit IgG antibodies were obtained from Life Technologies® and used at 1:2000 dilution. Luminata™ Forte Western HRP Substrate (EMD Millipore®, Billerica, Mass.) and CL-XPosure™ auto-radiography films (Thermo Fisher®) were used for chemiluminescent detection. Used membranes were stripped using a guanidine hydrochloride-based solution for re-probing with a different antibody. Uncropped scans of the immunoblots are shown in FIG. 17.

DNA Deamination Assay with Cell Lysates

The deamination assay described by Byeon et al. (Nat Commun 4, 1890 (2013)). was used. Whole cell lysates were prepared using M-PER™ reagent (Thermo Fisher®, Rockford, Ill.) with 1× Halt™ protease and phosphatase inhibitor cocktail (Thermo Fisher®). Briefly, 180 nM 5′ Alexa Fluor™ 488 fluorescent dye-labeled ssDNA substrate of 40 bases (Integrated DNA Technologies®) was incubated at 37° C. for an hour with 10 μl lysate and 10 units of E. coli uracil DNA glycosylase (New England Biolabs®) in 10 mM Tris (pH 8.0), 50 mM NaCl, 1 mM dithiothreitol (DTT) and 1 mM ethylene-diamine-tetraacetic acid (EDTA) in a volume of 50 μl. The reaction was stopped by adding 40 μg proteinase K (Life Technologies®) and incubating it for 20 minutes at 65° C. 10 μl of 1 N NaOH was added to the reaction which was then incubated at 37° C. for 15 minutes. After adding 10 μl of 1 N HCl, the reaction (10 μl) was electrophoresed on a 10% denaturing polyacrylamide gel. Typhoon™ 9400 Imager (GE Healthcare®) was used to scan the gel in fluorescence mode.

### Purification of Recombinant APOBEC3A Proteins

Rosetta™ 2(DE3) pLysS E. coli (EMD Millipore®) transformed with a bacterial expression construct for C-His6-tagged APOBEC3A and grown in Luria broth at 37° C. were induced for expression of the recombinant protein with 0.3 mM isopropyl β-D-1-thiogalactopyranoside (IPTG) and cultured overnight at 18° C. Harvested cells were lysed with a French pressure cell (American Instrument Corporation®, Hartland, Wis.) and Ni-NTA His.Bind Resin™ (EMD Millipore®) was used as per manufacturer's instructions to purify APOBEC3A protein from the lysates by affinity chromatography. Isolated protein was concentrated using an Amicon™ Ultra-4 Centrifugal Filter Unit with Ultracel-3 membrane (EMD Millipore® nominal molecular weight limit of 3 kDa). The concentrated protein was stored in 25 mM Tris (pH 8.0) with 50 mM NaCl, 1 mM DTT, 5% v/v glycerol, and 0.02% w/v sodium azide. Staining with Coomassie blue of protein preparation electrophoresed on a denaturing polyacrylamide gel indicated that it had APOBEC3A at >90% purity.

### In Vitro SDHB Editing Assay

Whole cell lysates of 293T transfectants were prepared using lysis buffer containing 0.2% Surfact-Amps™ NP-40 (Thermo Fisher®), 30 mM 4-(2-hydroxyethyl)-1-piperazine-ethane-sulfonic acid (HEPES; pH 7.5), 100 mM KCl, 25 mM NaCl, 1.5 mM MgCl2, 1 mM DTT, and 0.5× Halt™ protease and phosphatase inhibitor cocktail, and stored with 10% v/v glycerol at −80° C. SDHB ORF RNA of ˜1.1 kb was generated by in vitro transcription of XhoI enzyme-linearized plasmid DNA using reagents and methods provided with the MEGAscript™ T7 Transcription Kit (Life Technologies®). SDHB RNA isolated from the transcription reaction was treated with DNAse I (Thermo Fisher®), and its integrity verified by electrophoresis on an agarose gel. For in vitro SDHB RNA editing assay, transfectant cell lysate (2-8 μl containing 21-84 μg protein) was incubated at 37° C. for 4-11 hours with 50 pg (125 amole) of SDHB RNA in a buffer containing 0.02 U per μ1RNAse inhibitor (Protector™ Roche®), 100 mM KCl, 10 mM HEPES (pH 7.4), 1 mM DTT and 1 mM EDTA in a total volume of 50 μl. In vitro assays with purified APOBEC3A contained 5-10 μM APOBEC3A, 2-2.5 pM SDHB full-length RNA or single-stranded SDHB DNA (c.37-c.156), 10 mM Tris (pH 8.0), 50 mM KCl, and 10 or 100 nM ZnCl2 with or without 10 μM 1,10-phenanthroline (Sigma Aldrich®). The reactions were incubated for 2 hours at 37° C. RNA was purified from the reactions containing transfectant lysates or purified APOBEC3A using TRIzol™ (Life Technologies®) as per manufacturer's instructions. The c.136C>U editing of the exogenous RNA was assessed by allele-specific RT-PCR (Baysal et al., PeerJ 1, e152 (2013)) using a forward PCR primer (GGAATTCGGCACGAGGAC) (SEQ ID NO:3) that does not bind the cDNA of endogenous SDHB RNA. For Sanger sequencing to assess a 619 b segment of the RNA that spanned exons 1 to 5, the cDNA was amplified with primers with sequences GGTCCTCAGTGGATGTAGGC (SEQ ID NO:4) and TGGACTGCAGATACTGCTGCT (SEQ ID NO:5). For reactions with SDHB DNA as substrate, 4 μl of the reaction was directly used in PCR of volume 20 μl with primers with sequences TTGCCGGCCACAACCCTT (SEQ ID NO:6) and AGCCTTGTCTGGGTCCCATC (SEQ ID NO:7) to amplify the substrate for Sanger sequencing by the forward primer.

### Other

SDHB gene expression and c.136C>U RNA editing was quantified by RT-PCR. Unless noted otherwise, total RNA, genomic DNA and plasmid DNA were isolated using material and methods provided with TRIzol™, DNA Wizard Genomic DNA Purification Kit (Promega®), and Plasmid Kit (Qiagen®, Germantown, Md.), respectively. RNA/DNA was quantified by spectrophotometry on a Nanodrop™ 2000 instrument (Thermo Fisher®). Proteins were quantified using Bio-Rad® Dc™ assay with bovine serum albumin standards. Statistical tests were two-tailed and were performed using R 3.0, Excel™ 2010 (Microsoft®, Redmond, Wash.), or Prism™ 6.0 (GraphPad®, San Diego, Calif.) software.

### Results

### SDHB RNA Editing in IFN-Treated MEPs and M1 Macrophages

Similar to hypoxia, an IFN-rich microenvironment is another factor that monocytes are exposed to during inflammation. IFNs also up-regulate expression of APOBEC3 cytidine deaminases, candidate enzymes that may be responsible for the SDHB c.136C>U RNA editing observed in monocytes. We therefore examined whether interferons induce SDHB c.136C>U RNA editing. As shown in the left panel of FIG. 1a, treatment of MEPs with type 1 interferon (IFN1; 600 U per ml) or IFNγ (200 U per ml) for 24 hours induced SDHB c.136C>U RNA editing in MEPs, both in normoxia and hypoxia under 1% 02 (Mann-Whitney U test P<0.01, comparing untreated and interferon-treated samples). The editing level in normoxic or hypoxic MEPs was increased ˜6-fold by IFN1 and ˜3-fold by IFNγ, suggesting that the induction of RNA editing with IFN1 was higher than with IFNγ (Wilcoxon rank sum test P<0.03, comparing samples regardless of hypoxia treatment). An additive effect of interferons and hypoxia on SDHB c.136C>U RNA editing was observed, and this was confirmed in an independent experiment in which matched MEPs of seven individuals were cultured under normoxia or hypoxia with 0, 300 or 1,500 U per ml IFN1 for 24 hours. Editing level in cells treated with both hypoxia and IFN1 was higher than in cells treated with only hypoxia or IFN1 (FIG. 1a, right panel; Wilcoxon test P<0.02, for both concentrations of IFN1).

IFNγ is an inducer of M1 (pro-inflammatory) polarization of macrophages, which are derived from monocyte precursors. We therefore examined and compared SDHB c.136C>U RNA editing in basal, unpolarized (M0), M1 and M2 macrophages. M0 cells were derived in vitro from CD14+ peripheral blood monocytes, and matched M1 and M2 macrophages were generated from the M0 cells by treatment with IFNγ and lipopolysaccharides, and interleukin-4, respectively. The SDHB RNA editing was found to be absent in M0 macrophages but occurred at an average level of ˜27% in M1 cells (FIG. 1b, right panel). The editing level was significantly lower in M2 macrophages (˜2%), suggesting a strong induction of editing in macrophages by M1 but not M2 polarization.

### Wide-Spread RNA Editing in Hypoxic MEPs and M1 Macrophages

To investigate whether hypoxia affects editing of RNAs other than SDHB in MEPs, we performed RNA sequencing of matched normoxic and hypoxic MEPs of three healthy individuals. We also examined whole transcriptome RNA sequencing data obtained by Beyer et al. (PLoS One 7, e45466 (2012)) for matched M1 and M2 macrophages generated in vitro from peripheral blood monocytes of three individuals to determine whether M1 macrophage polarization differentially affects editing of other RNAs besides SDHB. Such comparison of whole transcriptomes of paired samples to identify RNA editing is less likely to falsely identify sequencing and mapping artifacts or genome sequence variations as RNA editing events.

About 84%-90% and 94%-97% of RNA sequencing reads of the MEPs and macrophages, respectively, could be uniquely mapped to the UCSC hg19 reference human genome (FIGS. 18-20). Calls made by the mapped reads for the reference base or a variation were counted along the genome, and paired count data were evaluated with the inverted beta-binomial test (Pham et al., Bioinformatics 28, i596-i602 (2012)) to identify genome positions at which the base variation level was differentially affected by hypoxia or M1 polarization with >2-fold change in either direction, with a q-value of <0.05 and a higher intra-group mean variation level of ≧5%. The type of RNA editing at a genome position was surmised from the base variation and the gene-coding chromosome strand at the position. The candidate RNA editing sites were filtered to remove likely false positives. Filtering criteria included identification of the site with a separate read-mapping software and location of the site within a known RefSeq gene (Methods and FIG. 21).

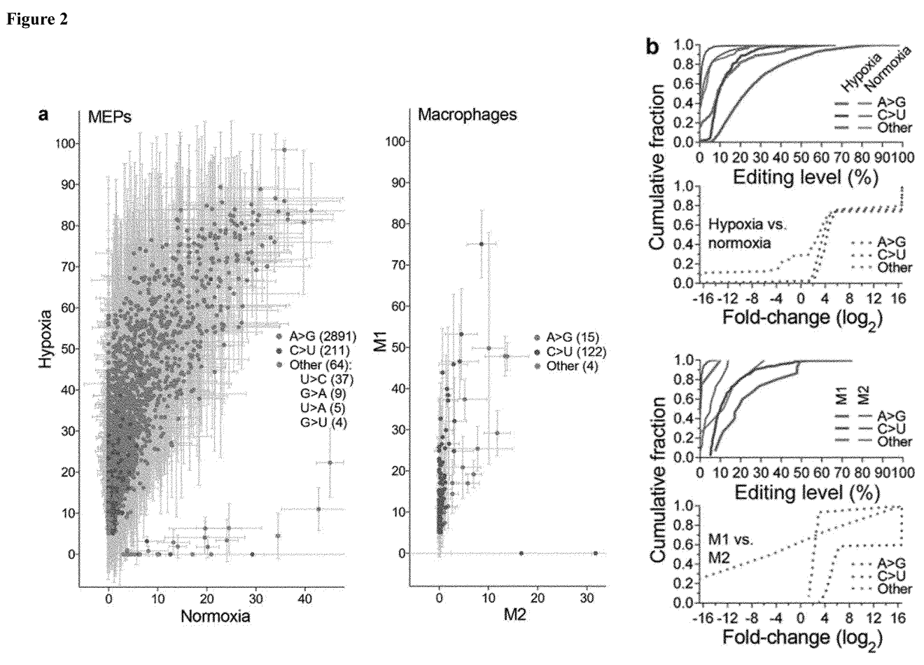

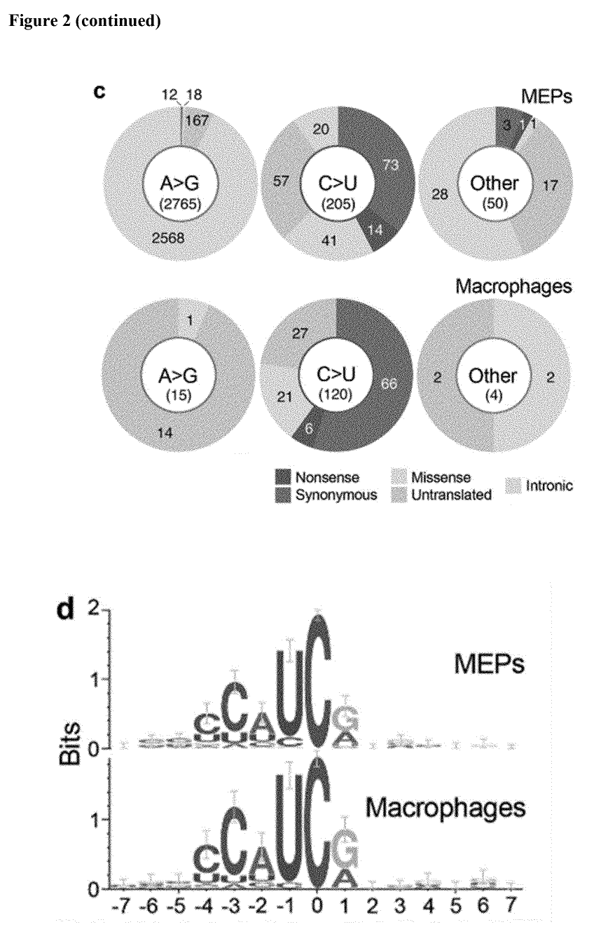

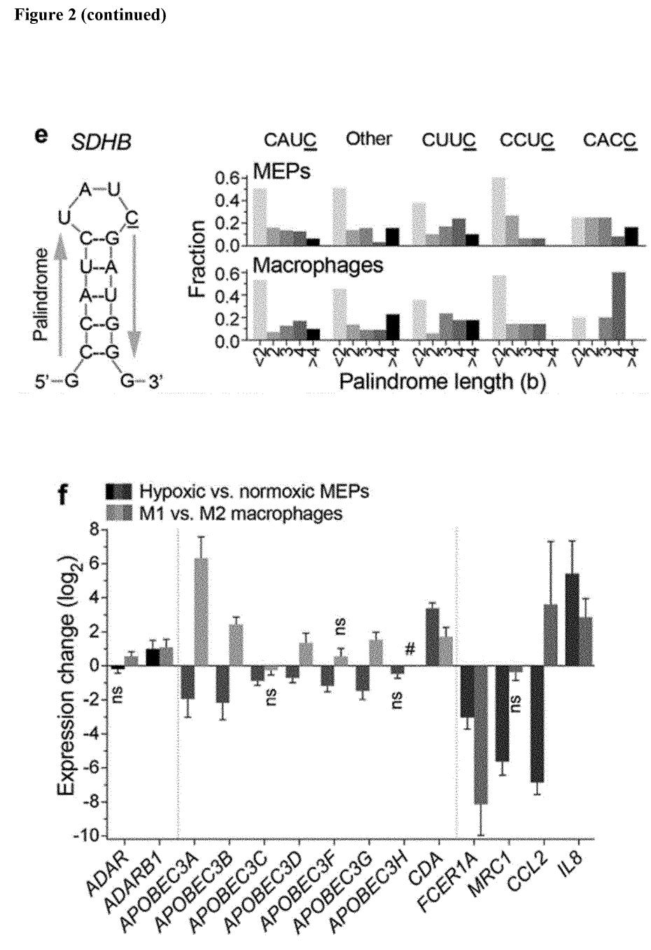

Putative RNA editing was found to be up- and down-regulated respectively at 3,137 and 29 sites by hypoxia in MEPs, and respectively at 139 and 2 sites by M1 compared to M2 macrophage polarization (FIGS. 2a and 2b, and Supplementary Data 1 in FIG. 22). Editing in MEPs was of A>I (A>G) and C>U types at 91.3% and 6.6% of the sites, respectively, whereas these two types of editing respectively occurred at 10.6% and 86.5% of the sites in macrophages. A>G editing occurred at an overwhelming majority of the sites in MEPs, but only 1.0% of the A>G sites were in coding exons, causing 18 non-synonymous and 12 synonymous codon changes (FIG. 2c and FIG. 22). This is consistent with the known targeting of A>G RNA editing to non-coding sequences. On the other hand, 61.7% of the total 211 C>U sites (in 199 genes) were in coding exons, causing 55 non-synonymous and 73 synonymous codon changes. C>U editing accounted for 73.3% of all non-synonymous editing up-regulated by hypoxia in MEPs. In macrophages, 77.9% of the total 122 C>U sites (in 116 genes) were in coding exons, causing 27 non-synonymous and 66 synonymous codon changes.

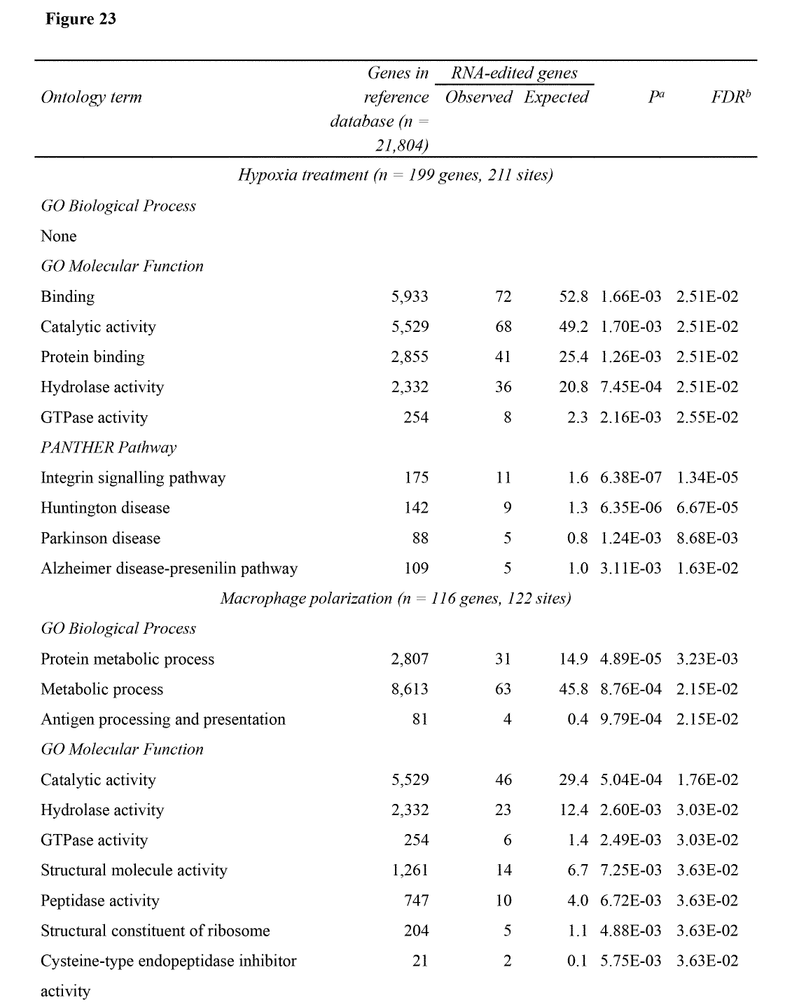

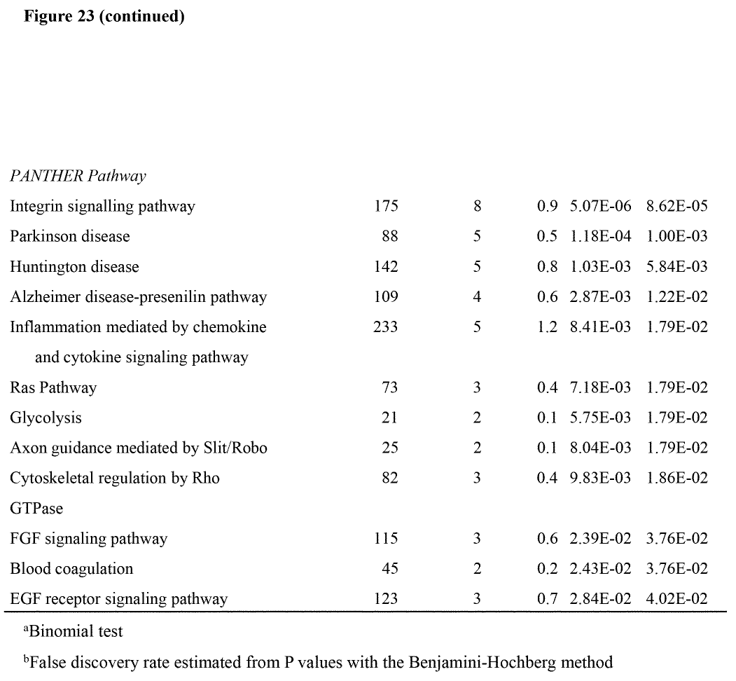

The average editing level in hypoxic MEPs was >10% and >20% for respectively 93 (45%) and 25 (12%) of the 206 C>U sites for which editing was up-regulated by hypoxia. In normoxic MEPs, the levels were <1% and <5% for respectively 162 (79%) and 202 (98%) of the 206 sites (FIG. 2b). Average C>U editing level in M1 macrophages was >10% and >20% for respectively 62 (51%) and 24 (20%) of the 122 sites. In contrast, levels in M2 cells were <1% and <5% for respectively 105 (86%) and 121 (99%) sites (FIG. 2b). Notably, 55 C>U RNA editing sites were shared by and upregulated in both the hypoxic MEPs and M1 macrophages. Editing of none of the 122 sites in M1 macrophages was down-regulated by hypoxia in MEPs. Ontology analysis of C>U RNA-edited genes of both MEPs and macrophages revealed enrichment for genes encoding for catalytic activities, and for genes in integrin-mediated signaling, and Alzheimer's, Huntington's and Parkinson's disease pathways (FIG. 23).

### Sequence and Structural Contexts of C>U RNA Editing Sites

C>U editing sites were most commonly present within a CCAUCG sequence motif (edited site underlined), with CAUC and its CACC, CCUC, CUUC and UAUC 1-nucleotide (nt) variants present for approximately 79% and 85% of the editing sites of MEPs and macrophages, respectively (FIG. 2d and Supplementary Data 1). Because the UAUC motif containing the SDHB c.136 nucleotide was flanked by palindromic sequences (FIG. 2e), we examined other C>U RNA editing sites to determine if the edited Cs in these were also flanked by palindromic sequences. About 51% and 52% of all edited NNNC sequences of MEPs and macrophages, respectively, were found to be flanked by short palindromic sequences of 2-7 nt (median=2 and 3 nt, respectively; FIG. 2e and Supplementary Data 1 in FIG. 23). Examination of minimum free energy structures of 60 nt sequences bearing the edited C in middle showed that the C residue was present in the loop of a stem-loop structure for 72% and 67% of the sites of MEPs and macrophages, respectively (Supplementary Data 1 in FIG. 22). These observations suggest that C>U RNA editing in MEPs and macrophages is catalyzed by cytidine deaminase(s) with particular target sequence and structure preference.

### Validation of Site-Specific C>U RNA Editing in MEPs

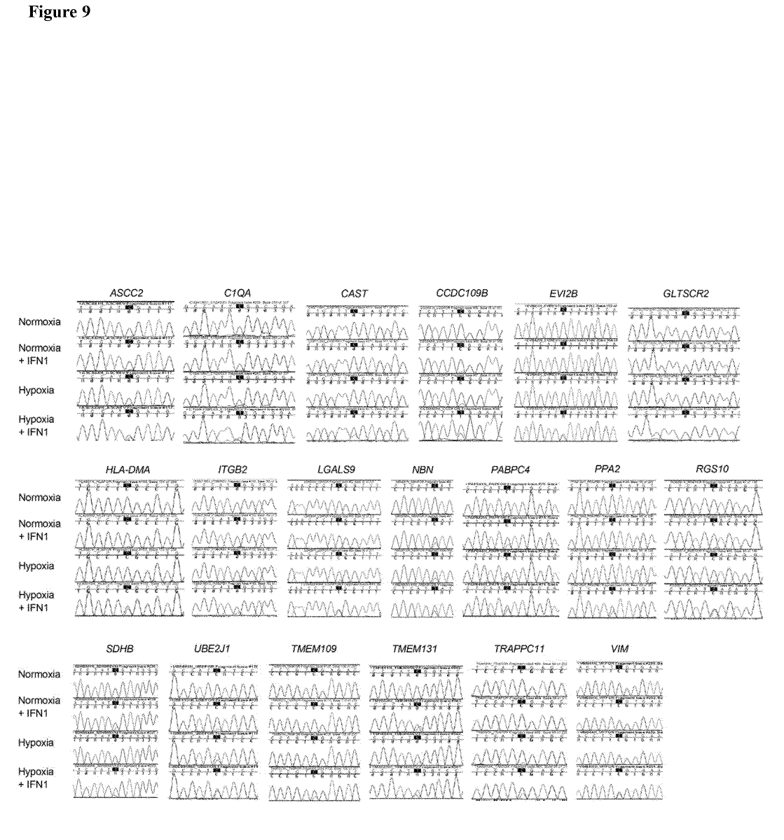

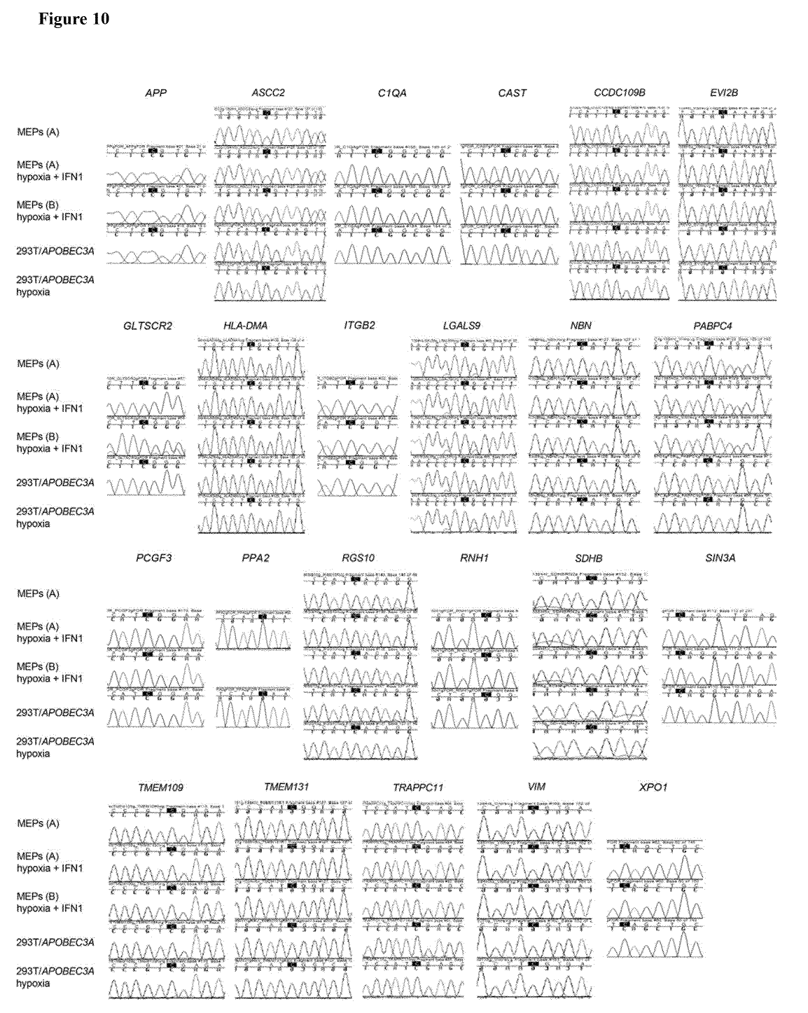

Thirty-three non-synonymous C>U RNA editing sites (in 33 genes) that were identified in analysis of RNA sequencing data (FIG. 2a) were chosen for experimental validation of site-specific editing by Sanger sequencing of RT-PCR products. Eighteen of the 33 sites were identified in MEPs, three in macrophages, and 12 in both (Table 1). RNA editing for 31 of the 33 genes, including the three exclusively identified in macrophages, could be experimentally validated in MEPs (Table 1). The RNA editing level for 19 genes was quantified in MEPs of three donors. Editing for none of the genes was observed in normoxic MEPs, but was seen for all in MEPs treated with hypoxia with or without IFN1 (FIG. 3a and FIG. 9). The additive effect of hypoxia and IFN1 on C>U RNA editing previously observed for SDHB (FIG. 1a) was also noticeable in the Sanger sequencing analyses for site-specific RNA editing of 18 other genes (FIG. 3a); the editing levels observed with combined hypoxia and IFN1 treatment (mean=38.2%) were significantly higher (Wilcoxon paired ranks test P<0.005) than the sum of those with IFN1 (mean=10.8%) or hypoxia (mean=14.5%) alone. Editing levels did not significantly differ between hypoxia and IFN1 treatments (ANOVA test P>0.05). Sanger sequencing of PCR-amplified genomic DNA fragments of hypoxia- and IFN1-treated MEPs did not reveal C>T nucleotide variation at the editing site for any of the 23 genes that were examined (FIG. 10).

MEPs contain both monocytes and lymphocytes. To determine the RNA editing levels of the 31 experimentally validated genes in these individual cell types, Sanger sequencing of RT-PCR products of monocyte and lymphocyte isolates (FIG. 11a) of hypoxia- and IFN1-treated MEPs of another three individuals was performed (FIG. 3b, and Supplementary FIGS. 11b and 11c). Editing levels in monocytes were more than in their parent MEPs, and >20% for 29 genes and >80% for five (TMEM131, 95%; SDHB, 90%; PCGF3, 90%; NBN, 84%; RNH1, 83%). In lymphocytes, RNA editing was seen for only two of the 34 genes (FAM89B and RHN1, ˜8% level for each), suggesting that most of the differential C>U RNA editing in MEPs occurred in the monocytes.

For two of the transcripts for which the editing results in a nonsense codon change, SDHB (NCBI reference sequence NM_003000, exon 2:p.R46X) and SIN3A (NM_001145357, exon 20:p.Q1197X), the effect of hypoxia-induced C>U RNA editing on protein level was examined by immunoblotting assays of whole cell lysates of monocytes isolated from normoxic or hypoxic MEPs of three donors in a separate experiment. As shown in FIG. 3c, hypoxia treatment of MEPs resulted in a significant reduction of both SDHB (280 amino acid residues, NCBI reference sequence NP_002991) and SIN3A (1,273 residues, NP_001138829) in monocytes. Hypoxia also reduced SDHB and SIN3A RNA levels by an average of 4.7- and 1.6-fold in these three CD14+ monocyte samples, as tested by quantitative RT-PCR (normalized against the B2M gene). While this reduction could be at the transcriptional level, it could also be a result of post-transcriptional processes such as nonsense-mediated decay and microRNA targeting of the transcripts because of the sequence change resulting from their editing.

APOBEC3A Expression is Associated with SDHB RNA Editing

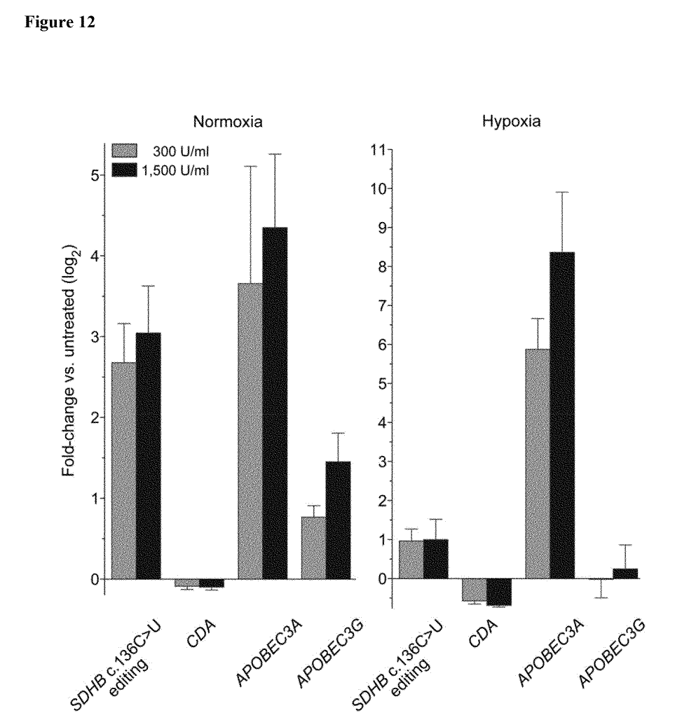

Next, we examined whether expression of any cytidine deaminase gene(s) was associated with SDHB c.136C>U RNA editing in monocytes and macrophages. CDA and the seven APOBEC3 genes were identified as expressed in RNA sequencing data of MEPs, and only CDA expression was up-regulated by hypoxia (FIG. 2f). Expression of APOBEC3A, the only APOBEC3 gene that is expressed at a higher level in monocytes compared to lymphocytes, was down-regulated by hypoxia. Expression of CDA and four APOBEC3 genes (A, B, D and G) was up-regulated in M1 compared to M2 macrophages, with APOBEC3A up-regulation being the highest (˜67-fold), whereas AID and APOBEC1, 2 and 3H were not expressed in macrophages (FIG. 2f). Up-regulation of APOBEC3A expression by IFN1 (also reported by Peng et al., J Exp Med 203, 41-46 (2006)), was seen in normoxic as well as hypoxic MEPs; IFN1 did not up-regulate CDA expression and up-regulated APOBEC3G expression only under normoxia (FIG. 12). Examination of changes in expression of cytidine deaminase genes in MEPs by hypoxia and IFN1, and in macrophages by M1 compared to M2 polarization, therefore suggested APOBEC3A and CDA as possible mediators of inducible C>U editing in MEPs and macrophages.

To further understand the association of cytidine deaminase gene expression with SDHB c.136C>U RNA editing, we evaluated RNA sequencing data in the Cancer Genome Atlas (TCGA) for three randomly chosen cancers, primary head and neck squamous cell carcinoma (HNSC), lung adenocarcinoma (LUAD), and secondary skin cutaneous melanoma (SKCM). Because tumors contain immune cells and can have hypoxic regions, we hypothesized that some degree of SDHB c.136C>U variation may be noticeable in the RNA sequences of the TCGA samples. Somatic SDHB c.136C>T mutation has not been identified in any TCGA sample for these cancers (data release 17 of International Cancer Genome Consortium (Hudson et al. Nature 464, 993-998 (2010)).

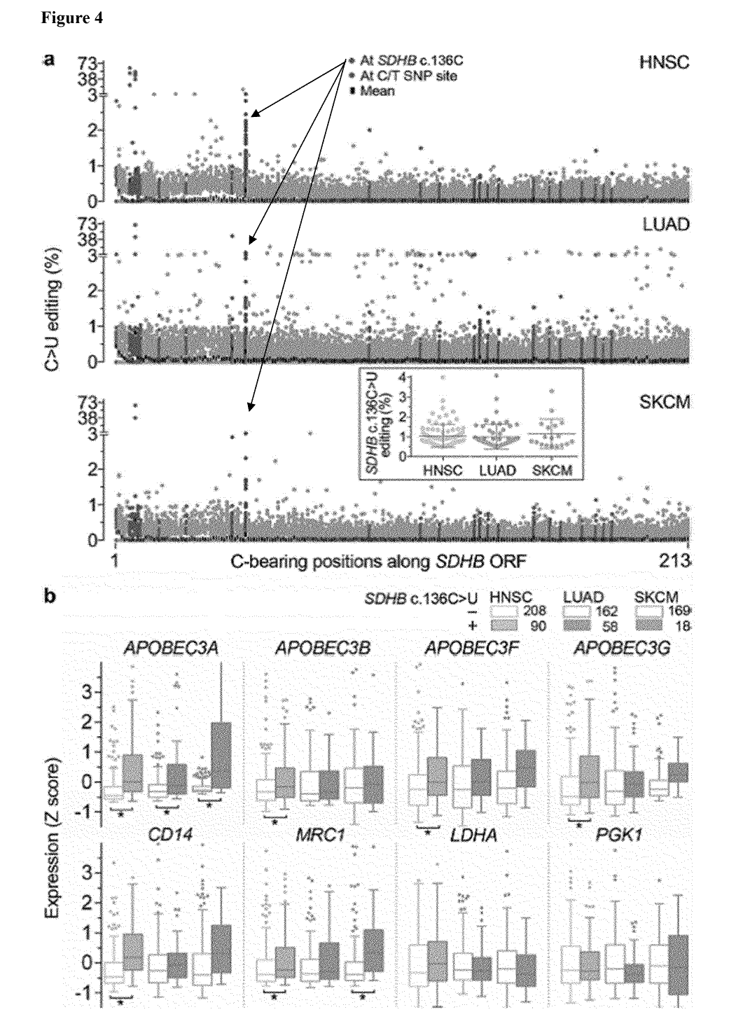

The scrutiny of TCGA's RNA sequencing data for the tumor tissues indicated putative C>U RNA editing of SDHB open reading frame (ORF) at c.136, but at no other site, in 30.2%, 26.4%, and 9.6% of the respectively 298 HNSC, 220 LUAD and 187 SKCM cases that were examined (FIG. 4a). The editing levels were low (˜1%), suggesting that it occurred only in a fraction of the cells of the tumors. Whole exome sequencing data for all eight tumors with editing level>2.25% showed complete absence of any sequence variation at c.136 at the genomic level (depth of coverage for SDHB c.136 ranging from 40-111, with mean=77). Comparison of gene expression between the editing-positive and -negative samples showed that APOBEC3A was the only cytidine deaminase gene whose expression was up-regulated in the editing-positive samples in all three cancers. Consistent differential expression of common hypoxia- or monocyte/macrophage-associated genes across all three cancers between editing-negative and -positive samples was not seen (FIG. 4b, and FIGS. 24 and 25).

### APOBEC3A Overexpression Causes C>U RNA Editing in 293T Cells

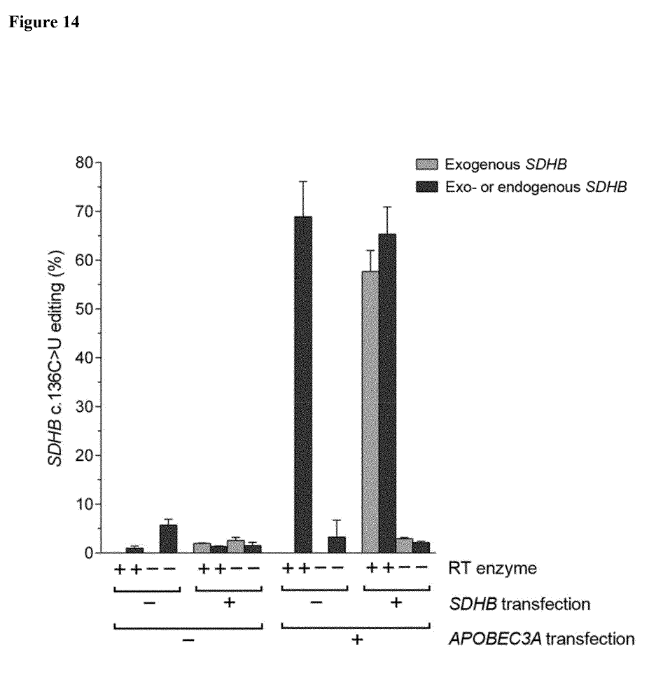

As noted above, the expression of APOBEC3A or CDA positively correlated the most with C>U RNA editing in cancer tissues, MEPs or macrophages (FIG. 25). To test if SDHB c.136C>U RNA editing can be induced by these two proteins, or by APOBEC3G whose expression is up-regulated by M1 macrophage polarization, their cDNAs were exogenously expressed in the human 293T embryonic kidney cell-line in which all three proteins were undetectable (FIG. 5a). Transient transfection of 293T cells for exogenous expression of APOBEC3A, but not APOBEC3G or CDA, induced SDHB c.136C>U RNA editing in the cells (FIG. 5b). Treatment of transfectants for 24 hours with hypoxia (1% 02) but not IFN1 (600 U per ml) mildly enhanced this editing (FIG. 5b). Previous studies have shown that intronic sequences are essential for A>I RNA editing, but not for APOBEC1-mediated C>U editing of APOB, which occurs in the nucleus after the APOB pre-mRNA has been spliced (Teng et al., Science 260, 1816-1819 (1993), Blanc et al., The Journal of biological chemistry 278, 1395-1398 (2003)). We found evidence for c.136C>U RNA editing of transcripts generated in vivo from a co-transfected, intron-less SDHB ORF cDNA expression construct in APOBEC3A transfectants, indicating that intronic sequences are not required for APOBEC3A-mediated RNA editing (FIG. 14).

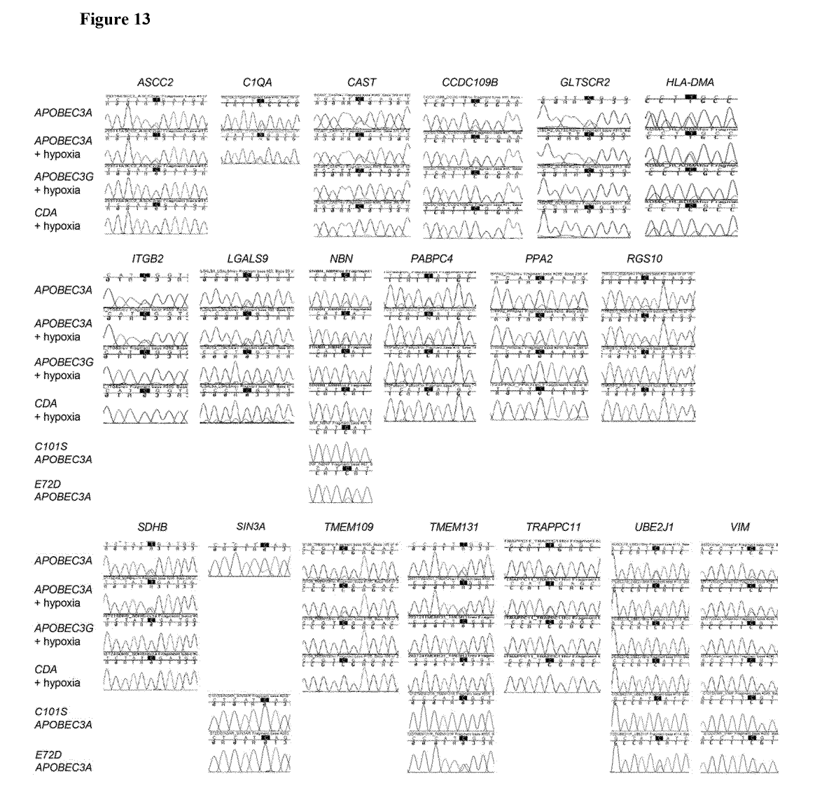

Sanger sequencing of RT-PCR products of the 293T transfectants showed that exogenous APOBEC3A, but not CDA, also caused site-specific C>U RNA editing for 30 genes for which RNA editing was previously validated for MEPs (editing for EVI2B could not be examined because of low gene expression; FIG. 5c and FIG. 13). This suggests that APOBEC3A mediates the transcriptome-wide C>U RNA editing that was noted for MEPs and macrophages (FIG. 2a). For most of the gene transcripts, hypoxia mildly increased the RNA editing levels, from an average level of 42% to 49% (Wilcoxon paired ranks test P=0.002; n=29). Sanger sequencing of PCR-amplified genomic DNA fragments of transfectants did not reveal C>T nucleotide variation at the editing site for any of the 23 genes that were examined (FIG. 10 and FIG. 5d). We tested the effect of APOBEC3A mediated RNA editing on the protein expression of 3 genes. Western blot assays of whole cell lysates of the transfectants for three proteins, ASCC2, SDHB and TMEM109, whose RNA transcripts were predicted to have p.R121X (in exon 4; NCBI reference sequence NM_032204, which encodes a protein of 757 aa), p.R46X (in exon 2; NM_003000, 280 aa) and p.R37X (in exon 2; NM_024092, 243aa) nonsense codon changes, respectively, because of RNA editing showed that exogenous APOBEC3A expression reduced levels of the proteins in 293T cells (FIG. 5d). In a separate RNA sequencing experiment, exogenous APOBEC3A expression in 293T cells was found not to affect SDHB RNA level in comparison to control transfectants, whereas it mildly but significantly affected ASCC2 and TMEM109 transcript levels, with fold-change values of ˜1.1 and 0.8, respectively. These results suggest that stop codons introduced by RNA editing may reduce wild type protein levels.

Notably, exogenous APOBEC3G also caused low-level, site-specific RNA editing for 11 genes in 293T transfectants (FIG. 6a); editing levels were highest for FAM89B and APP, for both of which the edited cytidine residue occurs in a CC sequence context that is known to be preferred by APOBEC3G for DNA deamination (Holtz et al., Nucleic acids research 41, 6139-6148 (2013)).

### APOBEC3A Knock-Down Reduces RNA Editing in M1 Macrophages

To validate that APOBEC3A mediates SDHB c.136C>U RNA editing in M1 macrophages (FIG. 1b), we transfected M0 macrophages with small interfering RNA (siRNA) at 100 nM to knock down APOBEC3A RNA, induced their M1 polarization after a day, and examined the M1-polarized cells after another 24 hours. Transfection of cells with either of two different siRNAs predicted to target APOBEC3A, or their equimolar mix, led to a significant reduction in APOBEC3A transcript and APOBEC3A protein levels compared to cells transfected with a control siRNA that is not predicted to target APOBEC3A (FIGS. 6a and 6b). There was no effect on APOBEC3G RNA level in the cells, suggesting that the knock-down was gene-specific (FIGS. 6a and 6b). Reduction of APOBEC3A RNA level was associated with a significant reduction of SDHB c.136C>U RNA editing (FIG. 6c), indicating that APOBEC3A is a major determinant of this editing in M1 macrophages. Sanger sequencing of RT-PCR products was used to evaluate site-specific C>U editing level for transcripts of five other genes for which RNA editing in M1 macrophages had been noted in analysis of transcriptome sequencing data (FIG. 2a). Examination of the sequence chromatograms showed that macrophages transfected with an siRNA predicted to target APOBEC3A had a lower level of RNA editing for all five genes compared to cells that were transfected with the control siRNA (FIG. 6d).

### RNA Editing by APOBEC3A Variants and Retrotransposition

The C101 residue of APOBEC3A is critical for binding of zinc, and the C101S APOBEC3A mutant completely lacks deamination activity against cytidines of ssDNA in vitro (Chen et al. Curr Biol 16, 480-485 (2006), Mitra et al. Nucleic Acids Res 42, 1095-1110 (2014)). As expected, cell lysates of the 293T transfectants exogenously expressing this mutant (FIG. 7a) did not cause deamination of the single cytidine residue of an ssDNA 40-mer (FIG. 7b). To test if C101 residue is essential for the observed RNA editing, we transfected 293T cells with the mutant cDNA. SDHB c.136C>U RNA editing, or site-specific C>U RNA editing for five other examined genes for which editing was observed in transfectants expressing the wild-type APOBEC3A was abolished in the C101S APOBEC3A transfectant (FIG. 7c and FIG. 13). The E72D and P134A variants of APOBEC3A were previously shown to variably impair the ssDNA deamination activity of the wild-type enzyme (Mitra et al., Nucleic Acids Res 42, 1095-1110 (2014)). We found that whole cell lysate of 293T transfectant of E72D, but not P134A, was moderately impaired in the ssDNA deamination assay (FIGS. 7a and 7b). Unlike for C101S, the E72D variant was capable of C>U RNA editing of transcripts for SDHB and five other genes that were examined, though to lesser levels than the wild-type protein (FIG. 7c and Supplementary FIG. 5). The SDHB RNA editing level in transfectants of the P134A variant was ˜80% of that of transfectants expressing the wild-type APOBEC3A (FIG. 7c). These results suggest that the catalytic activity required for DNA deamination by APOBEC3A is also important for RNA editing.

APOBEC3A suppresses retrotransposition in cell-based assays and this suppression is dependent on its ssDNA cytidine deaminating catalytic integrity (see discussion). To test whether RNA editing and retrotransposition suppressing functions of APOBEC3A are linked, we tested the effect of mutations on LINE1 (L1)-retrotransposition. We found that the ability of the E72D, C101S and P134A variants to inhibit retrotransposition paralleled their RNA editing activities (FIG. 7d). These findings indicate that mutations in E72, C101 and P134 residues of APOBEC3A affect the protein's ssDNA and RNA deamination, and anti-L1 retrotransposition activities in a similar manner.

In Vitro Deamination of SDHB RNA and ssDNA by APOBEC3A

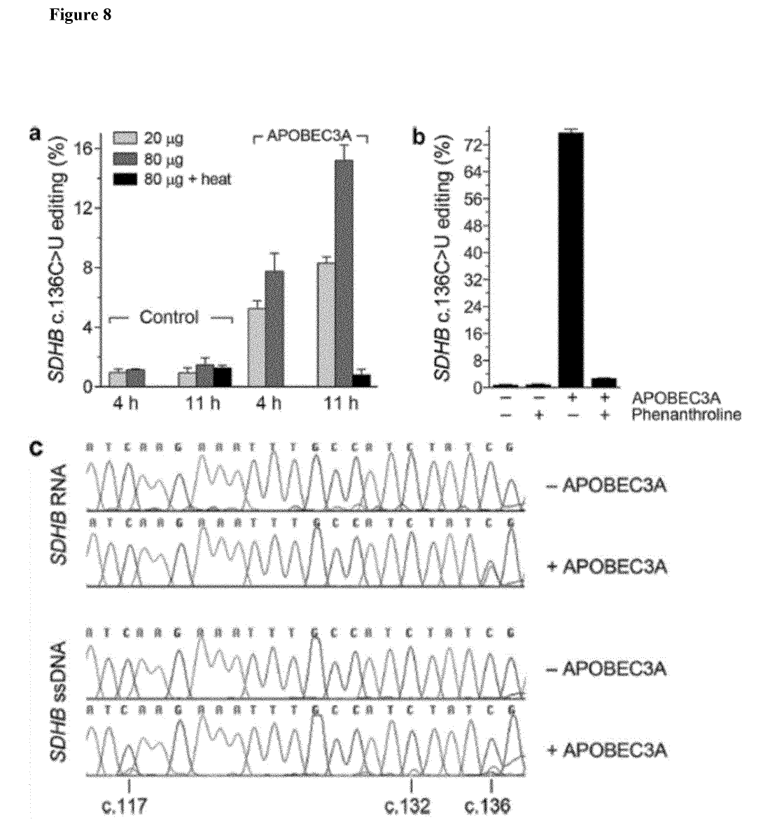

The various observations thus far noted suggest that APOBEC3A can deaminate cytidines in RNA. To demonstrate that APOBEC3A can edit c.136C>U in SDHB RNA in vitro, an SDHB ORF RNA of ˜1.1 kb with an artificial sequence at its 5′ end was incubated with whole cell lysates of 293T transfectants. Editing of the RNA at c.136 was quantified by allele-specific RT-PCR with a 5′ primer that was specific to the artificial sequence and using the same 3′-primers as described (Baysal et al., PeerJ 1, e152 (2013)). Lysate expressing APOBEC3A but not a control transfectant induced C>U editing of the exogenous SDHB RNA at c.136 in a time- and dose-dependent manner, and this activity was not seen with the heat-inactivated lysate (FIG. 8a). To further validate the RNA editing activity of APOBEC3A, in vitro editing assays were performed with purified APOBEC3A. Incubation of in vitro transcribed SDHB RNA with His6-tagged APOBEC3A protein showed site-specific editing of the SDHB RNA in vitro (FIG. 8b). Chelation of zinc in the deamination reaction with 1,10-phenanthroline abolished the editing (FIG. 8b). An ssDNA of 120 bases containing the SDHB cDNA sequence (c.37-c.156) too was deaminated at c.136 by the recombinant APOBEC3A protein. However, cytidine deamination of the ssDNA was also observed at other positions (c.117 and c.132); the deaminated residue at both positions occurs in a TC sequence context (FIG. 8c). In contrast, cDNAs of the in vitro synthesized SDHB RNA incubated with the APOBEC3A 293T transfectant cell lysates or the pure recombinant enzyme showed no evidence of additional mutations in Sanger sequence analysis of a 619 b segment that spanned exons 1 to 5 (FIG. 8c). Thus, whereas cytidines of both SDHB ssDNA and RNA can be deaminated in vitro by APOBEC3A, deamination sites of RNA appear to be highly selective which may reflect a requirement for a more complex sequence or structure context.

### Discussion

In this study, we demonstrate that APOBEC3A, a cytidine deaminase highly expressed in myeloid cells, is a C>U RNA editing enzyme that modifies the monocyte/macrophage transcriptome. The RNA editing in monocytes is activated by hypoxia and interferons in both independent and additive manners (FIGS. 1a and 2a), and in monocyte-derived macrophages by M1 but not M2 polarization (FIGS. 1b and 2a). These findings represent the discovery of the first mammalian RNA-editing cytidine deaminase enzyme since the identification of APOBEC1 in 1993, unveil a previously unrecognized function for the APOBEC3 family of genes, markedly expand our knowledge of C>U RNA editing events, and highlight a significant effect of micro-environmental factors on such editing.

The RNA editing activity of APOBEC3A (FIG. 8b) provides a new perspective to understand the anti-viral and -retrotransposition functions of APOBEC3A and possibly other APOBEC3 genes. APOBEC3A has been shown to strongly inhibit retrotransposons and diverse viruses including parvoviruses, alpharetroviruses, HTLV-1 and HIV-1 in the early stages of infection in myeloid cells (Ooms, et al., Journal of virology 86, 6097-6108 (2012), Arias et al., Front Microbiol 3, 275 (2012), Wiegand et al., Journal of virology 81, 13694-13699 (2007), Berger et al., PLoS pathogens 7, e1002221 (2011)). The mechanism by which APOBEC3A inhibits these agents is poorly understood. We find that the RNA editing and anti-LINE-1 retrotransposition abilities of APOBEC3A are similarly affected by E72D, C101S and P134A mutations (FIGS. 7c and 7d). This is consistent with the possibility that the newly discovered RNA editing activity of the host RNAs by APOBEC3A may provide a DNA deamination-independent mechanism for the inhibition of viruses and retrotransposons by the protein. The association established in this study between up-regulation of APOBEC3A-mediated C>U RNA editing of cellular transcripts and hypoxia or interferon-treatment of monocytes and M1 polarization of macrophages (FIGS. 1 and 2a) also supports this notion.

Non-synonymously C>U RNA-edited genes identified in this study may represent players that mediate the anti-viral and -retrotransposition function of APOBEC3A.

APOBEC3A is believed to deaminate foreign but not host genomic DNA in primary cells, and previous studies have demonstrated the deamination activity of the enzyme against ssDNA but not RNA (Mitra et al., Nucleic Acids Res 42, 1095-1110 (2014), Stenglein et al., Nat Struct Mol Biol 17, 222-229 (2010). Our data (FIG. 2d) indicates that the enzyme deaminates cytidines of RNA within CAUC or its 1-nt. variant motifs that are flanked by palindromic sequences. It thus appears that previous studies failed to observe the RNA editing activity of APOBEC3A, which is known to bind RNA, in part because they used substrate RNAs containing a non-specific sequence.

An important finding of this study is that hypoxia independently activates C>U RNA editing to levels comparable to those induced by IFN1 (FIGS. 1a and 3a). Moreover, stimulation of MEPs by hypoxia and IFN1 together additively increases editing, with levels reaching over 80% for five of the 31 genes validated by Sanger sequencing (FIG. 3a). Since hypoxia is pervasive in inflamed tissue, this suggests that RNA editing has the potential to substantially alter certain cellular proteins in virus-infected cells in vivo. How hypoxia activates C>U RNA editing is currently unknown. Although up-regulation of APOBEC3A expression may underlie the activation of C>U RNA editing by interferons (FIGS. 1a and 3a), APOBEC3A expression in MEPs is down-regulated by hypoxia (FIG. 2f). Hypoxic stimulation of C>U RNA editing in these cells may therefore be caused by an alternative mechanism such as enhanced translocation of the enzyme to nucleus, where A>I and APOBEC1-mediated C>U RNA editing are known to occur. Monocytes routinely encounter hypoxia upon their exit from the highly oxygenated bloodstream to inflamed tissues, but the oxygen-sensing mechanisms in these cells are poorly understood. We find that RNAs encoding for both the SDHA and SDHB subunits of mitochondrial complex II are targets of hypoxia-induced C>U editing (Supplementary Data 1 in FIG. 22), suggesting that suppression of this complex facilitates hypoxia adaptation in pro-inflammatory monocytes and macrophages.

Monocytes and monocyte-derived pro-inflammatory macrophages play an important role in pathogenesis of common diseases including infectious diseases, obesity, cancer, Alzheimer's disease and atherosclerosis. We found that APOBEC3A causes non-synonymous RNA editing of transcripts of the APP, AP2A1, CAST, LRP10 and XPO1 genes (FIG. 5c) that are implicated in pathogenesis of Alzheimer's disease through regulation of amyloid precursor protein. Analyses of RNA sequencing data of this study shows that up-regulation of CD33 gene expression, which is associated with AD susceptibility, also occurs in MEPs under hypoxia (3.1-fold, FDR=0.0002, Fisher's exact test), and in M1 relative to M2 macrophages (2-fold, FDR=0.012, Fisher's exact test). It is thus possible that inflammation and hypoxia create multiple risk factors for chronic diseases like Alzheimer's disease through RNA editing and altered gene expression in monocytes/macrophages.

Our findings reveal an unprecedented extent and level of protein-recoding RNA editing in innate immune cells in response to certain micro-environmental factors associated with inflammation, which is mediated by APOBEC3A. In the light of important role which APOBEC3A plays in restricting diverse viruses and retrotransposons, these findings suggest a deaminase-dependent cellular RNA editing model which can be used to investigate the molecular bases of these restrictions and to identify agents that affect RNA editing.

RNA Sequencing data of MEPs were deposited in NCBI Sequence Read Archive (SRA) with accession number SRP040806.

TABLE 1
Candidate sites experimentally examined for validation
of C > U RNA editing^{a}

| | | | Editing level (%)^{d} | | | |
|---|---|---|---|---|---|---|
| | Chromosomal | cDNA and amino | MEPs | | Macrophages | |
| Gene | position^{b} | acid change^{c} | Normoxia | Hypoxia | M2 | M1 |
| AP2A1 | 19: 50295238 | C520T, R174X | 0 | 15.5 | NA | NA |
| APP | 21: 27326988 | C1546T, R516C | 0 | 8.5 | NA | NA |
| ASCC2 | 22: 30221126 | C202T, R68X | 0.9 | 19.1 | NA | NA |
| C1QA | 1: 22965523 | C361T, R121W | NA | NA | 0.1 | 10.7 |
| CAST | 5: 96106257 | C1826T, S609F | 0 | 7.2 | 0.1 | 5.2 |
| CCDC109B | 4: 110605624 | C638T, S213L | 0.3 | 20.4 | NA | NA |
| EVI2B | 17: 29632509 | C119T, S40L | 0 | 18.1 | 0.6 | 12.1 |
| FAM89B | 11: 65340979 | C437T, P146L | 0 | 16.2 | NA | NA |
| GLTSCR2 | 19: 48253494 | C349T, R117W | 0.5 | 6.8 | 0.2 | 11.2 |
| GPR160 | 3: 169801777 | C17T, S6L | 0.6 | 15.7 | NA | NA |
| HLA-DMA | 6: 32918428 | C241T, R81C | 0.6 | 9.9 | NA | NA |
| ICAM3^{e} | 19: 10444896 | C1381T, Q461X | 0 | 18.4 | 0 | 7.2 |
| ITGB2 | 21: 46319067 | C908T, S303L | 0.9 | 5.1 | NA | NA |
| LGALS9 | 17: 25967659 | C193T, R65W | 0.2 | 7.6 | 0 | 5.4 |
| LRP10 | 14: 23346296 | C1702T, R568X | 0 | 6.2 | NA | NA |
| NBN | 8: 90955531 | C2134T, H712Y | 4 | 24.2 | 0 | 22.4 |
| PABPC4 | 1: 40027426 | C1840T, H614Y | 4.4 | 31.7 | 1.6 | 39.9 |
| PCGF3 | 4: 737366 | C367T, R123W | 2 | 22.2 | 0 | 12.8 |
| PPA2 | 4: 106317458 | C319T, Q107X | NA | NA | 0.2 | 8.8 |
| PRPF40A | 2: 153515789 | C2404T, R802X | 0 | 5.1 | NA | NA |
| RGS10 | 10: 121275109 | C311T, S104L | 0.2 | 7.9 | 0 | 15.3 |
| RNH1 | 11: 499165 | C464T, S155L | 3 | 18.5 | 0 | 9.2 |
| SDHB | 1: 17371320 | C136T, R46X | 2.6 | 23 | 1.1 | 15.6 |
| SIN3A | 15: 75668008 | C3589T, | 0 | 17.2 | NA | NA |
| | | Q1197X | | | | |
| SETX^{e} | 9: 135201977 | C5008T, | 1.9 | 24.6 | NA | NA |
| | | Q1670X | | | | |
| SUPT6H | 17: 27005584 | C1138T, R380X | 0.6 | 12.3 | NA | NA |
| TMEM109 | 11: 60687274 | C109T, R37X | 0 | 11.2 | NA | NA |
| TMEM131 | 2: 98409343 | C3650T, S1217L | 5.7 | 26.4 | NA | NA |
| TMEM179B | 11: 62556843 | C364T, R122X | NA | NA | 0 | 5.2 |
| TRAPPC11 | 4: 184585120 | C100T, R34X | 0 | 15.8 | NA | NA |
| UBE2J1 | 6: 90048208 | C292T, H98Y | 4 | 16.1 | 1.1 | 18.9 |
| VIM | 10: 17277300 | C1141T, R381C | 0.3 | 15.7 | NA | NA |
| XPO1 | 2: 61760990 | C43T, Q15X | 2.7 | 10 | NA | NA |

^{a}NA, either editing level was not different between the two groups of samples or it could not be determined
^{b}Based on the UCSC hg19 genome assembly used for mapping reads with the Subread subjunc aligner
^{c}Nucleotide numbering for the shortest transcript isoform, with A of the ATG translation initiation codon at position 1
^{d}Calculated in analysis of RNA sequencing data (Supplementary Data 1); mean value (n = 3)
^{e}Failed Sanger sequencing-based experimental validation

The following sequences are listed in the figures:

| Sequence | ID No. |
|---|---|
| AGCCTCGCCTTTGCCGA | 8 |
| TGACATCCCCCGCATCCTGG | 10 |
| CACACATATTCACTTCCAACTTTAAC | 12 |
| GGCCGAGGACCCGAAGG | 14 |
| ATGTCCGCGCAGAACAGAA | 16 |
| CTTTGACGAGACTCTACAGAAG | 18 |
| AGGCAGGAAGACCTGGCAGA | 20 |
| AAACCTGCAGATGACCAAGAC | 22 |
| AGAGAGAGCACCATTTACTG | 24 |
| CTCACCCAGGAGGGGAGAATC | 26 |
| GCAAAAGAAGTTAACAGCTGAG | 28 |
| TCAACCTCGACTCAGCGCTG | 30 |
| TTGTTGTCAGAGGCCCCA | 32 |
| GGAAGATCATCAGTCAAGGAAG | 34 |
| TGGCTGCTACCCCACTCCTG | 36 |
| AATCGGCTGGCGCAACGTCA | 38 |
| CTCAGCTCCAGTGGAACCAG | 40 |
| CACCTGCAAGCTCTATGCCAT | 42 |
| AAATCCATCTGGCATAAATGATGA | 44 |
| GGGAATGCTGCTGGAGATAG | 46 |
| TGGTACCAGGCCTCCAAG | 48 |
| GATGATGTTAAGAAGTTCAAACC | 50 |
| GAGGACATAACTCTAGAATCTG | 52 |
| GAAAATGCAAGATAAGACGCAG | 54 |
| GCTGCACCTCAGCGACAAC | 56 |
| TGTGGATAGTCTGGATAAGCT | 59 |
| TGATTACCTAGACCGAGGGC | 61 |
| ATAGCAGTGGCCTGAGAAAG | 63 |
| ATCTCAGGAGAATCGTTGGT | 65 |
| GGCGATGACTCGGACCCAG | 67 |
| TGTGACATCGTAAACATGAG | 69 |
| CCAGACTCCGATTTTGATGGAG | 71 |
| GCAGAAGAATGGTACAAATCCA | 73 |
| GCATCAGCAAGAGCAACAG | 75 |
| CTGGTGCCTGGGGCG | 9 |
| CATGTGCTGGTCATTGAGCAG | 11 |
| GTCCAGGCGCTCCACTTC | 13 |
| TTCTGACACAGGCTGCGAAG | 15 |
| CAACTTCATCCTGAATCTCCT | 17 |
| AAAGAGCACGCAGAGGTCCAG | 19 |
| GGGTACAGTGCAGACGAATC | 21 |
| AGTCATCTTTTGGCTTGGAAG | 23 |
| ATATCCCAGGAGTACACCCA | 25 |
| CGATAGCAATTGCCCTGAAATCC | 27 |
| GTTGGTTGTCCAGCAGTGACT | 29 |
| GATGTGGAAGGCATCCTGCA | 31 |
| TCCTTCCGCCTGAGCTTCTT | 33 |
| CCCAAGTATGATCAAGAATAGC | 35 |
| GCCCTATTTGCTGGATCATCC | 37 |
| CTCGTAGGTCTTCACCATCC | 39 |
| AGGATCCCGTTCACCATCAC | 41 |
| CAAGGGGAGCAGCAGAAGG | 43 |
| CTCCATTTCCTGCCTTAGCC | 45 |
| TCAAACCTTGAGTTGAATTCCATA | 47 |
| CTGCGGTGGTAGTCGTTGTC | 49 |
| ATCTTGCTTCCTCTTGAGTGCA | 51 |
| GATTTATGTCTCCTCTTCTTAC | 53 |
| CTGGTCCTGGAGTTTCTGGA | 55 |
| CTGAGCCTGGAGCTGGGGT | 57 |
| GTCAAAGTAGAGTCAACTTCAT | 58 |
| GCACATGCTCCTTGGTCCA | 60 |
| ATGCCATCAGCAAGAGGTTTGT | 62 |
| CTCTGACACCAGGTGCATGG | 64 |
| AGCAGACTGTGAAGAAGCTGCT | 66 |
| CCAAATTCACCCAAGAAGAG | 68 |
| GGAATGTACCACTCATATGAAG | 70 |
| GCTCTTCTTTCCTCAGGAGTG | 72 |
| CTGTAGGTGGCAATCTCAATG | 74 |
| TCAGTACTTCTTGAGCCATTC | 76 |
| GCAAGAGTTCCTTTTCATCTTGC | 78 |
| CTTTCATTATAAACCCGCTATAG | 79 |
| TCACCCTGTGTCCTTTGCAG | 80 |
| GAAGACTATTTGCTTTCCCTGG | 81 |
| TATGCATGATTTACCATCTTTGC | 83 |
| GTTGGCTTTGGTCCACCAC | 84 |
| CTTTCTCTTGCCACAGCAGCT | 86 |
| CCAGCAAAATGGAATTATCTTGT | 87 |
| GCACTCAGCTCACTGTGCTT | 89 |
| CAGTTGGTCCCTTTTTCAGC | 90 |
| TGCATTGTTTATTCCTCAGGC | 92 |
| CACCAGACATCTTTCTCACC | 94 |
| GTCATGGGATCAGTGGCTTAC | 77 |
| GGAGGGGATTCTTGCTCAC | 82 |
| TGAAAATAGCCACACATACGG | 85 |
| CTCTCCTTCAATAGCTGGCTT | 88 |
| AACACATGCCATCACAATGCC | 91 |
| GCTTTGTAAAAGCAACTGGGT | 93 |
| ACGCATCCAAGTCTGAGTTCC | 95 |
| GCATCCGGACAGGCATCCAA | 96 |
| GCCATGCGGACCTGCGCTTCT | 97 |
| CTGGAGCCCCAGCTTCTCCTG | 99 |
| TGATTACGACGCCCTATATAAGGAGG | 101 |
| GGCCGTAAAAGCCACAGAG | 98 |
| GAGATGAACCAAGTGACCCTG | 100 |
| TAGATGCGGGCAGCGAAG | 102 |

### Example 2

This examples demonstrates identification of APOBEC3G as an RNA editing enzyme. In Example 1, we describe that A3A concordantly induces widespread site-specific C>U RNA editing of cellular transcripts in proinflammatory macrophages and in monocytes exposed to hypoxia and/or interferons. We also show that RNA editing function of A3A can be recapitulated by transient overexpression in 293T cells which causes site-specific RNA editing of thousands of genes (in revision). In this example, To explore whether A3G is capable of RNA editing, we transiently overexpressed it in 293T cells, performed transcriptome-wide sequencing and analysis and performed targeted experiments. We found that A3G is capable of RNA editing of a distinct set of genes, including some linked to HIV-1 replication as host factors.

RNA Seq Analysis and Verification

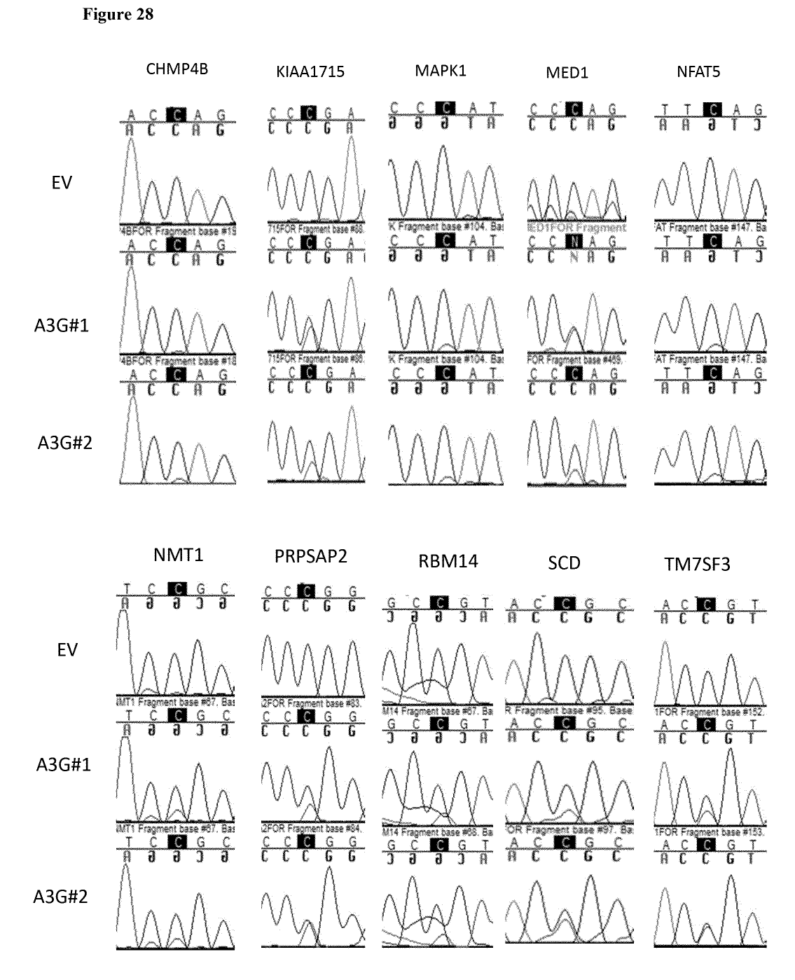

To examine transcriptome-wide RNA editing events of APOBEC3G, we transfected 1 μg of pA3G into 293T cells (293T/A3G) which caused robust protein expression (FIG. 28A). Control transfection with A3A (1 μg) showed high levels of protein expression and RNA editing as tested by one of the highly edited sites at SDHB c. 136C>U (mean RNA editing levels=50.55%±SD compared to 0.87%±SD with control empty vector, n=3). To identify transcriptome-wide RNA editing sites for A3G, we performed RNA seq approach comparing the sequences of 293T/empty vector (control) and 293T/A3G transcriptomes. 37-71 million reads were obtained for each sample in RNA sequencing. The average depth of coverage by mapped reads among the samples was at least 9 for 28-31 million genomic nucleotide positions. These positions were examined for RNA sequence variation.

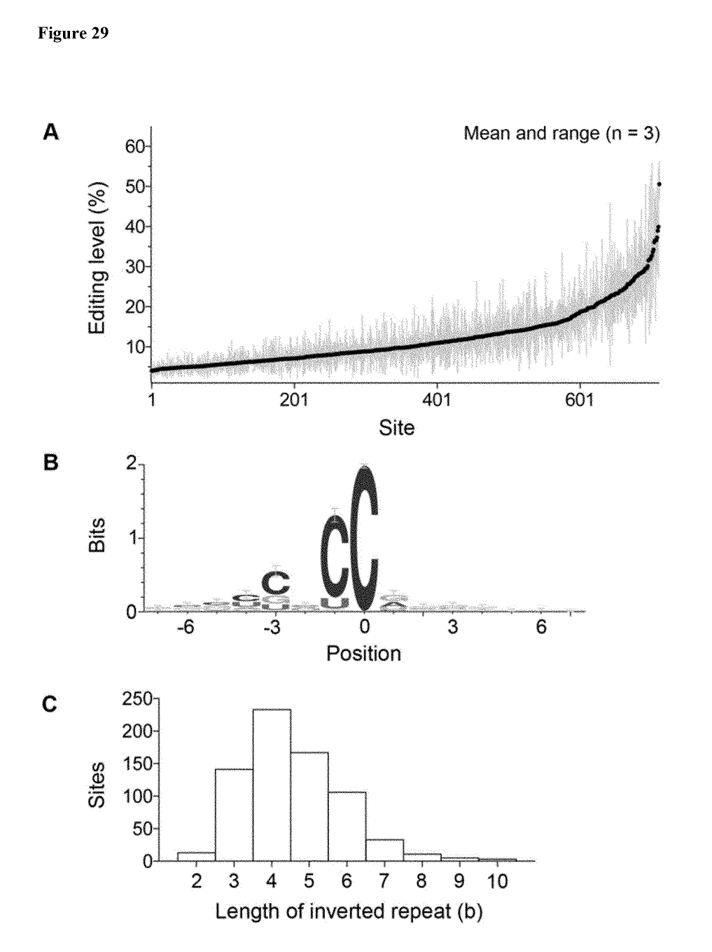

In analyses of RNA sequencing data to identify single-nucleotide sequence variations, significant differences in RNA sequences of the two groups of A3G and control transfectants were identified for 712 genomic positions. At all these positions, the sequence variation was of C>U but not any other type. Average levels of such putative C>U RNA editing were 0 in all the control samples and >5% in all the APOBEC3G transfectant samples for all 712 sites. Average C>U RNA editing levels in the A3G transfectant samples at the 712 sites were between 4% and 51% (mean=11%, SD=7%). Editing level was >20% and >30% for respectively 86 (12%) and 15 (2%) sites (FIG. 29).

690 (97%) of the 712 sites occur in the known human (RefSeq) transcriptome. Of these 692 sites, 405 (59%) are in known exonic RNA sequences (table X). C>U editing of RNA at the 712 sites is predicted to result in 174 (24%) synonymous, 173 (24%) missense and 48 (7%) nonsense changes in RNA translation (Table 2). Protein recoding RNA editing occurred in 221 sites in 217 genes. The 690 editing sites that are in the known transcriptome are transcribed for a total of 635 genes. The higher number of editing sites (4) was seen for two genes, HCFC1 and IGF2BP1. Two and 48 genes respectively had 3 and 2 editing sites (table X). Correlation between editing and gene expression levels was not observed. Notably, C>U recoding RNA editing at 27 sites in 27 genes was also catalyzed by A3A which causes such recoding of 1,100 genes in the 293T overexpression system. This finding suggests that A3A has a broader target gene profile than A3G and that RNA editing targets of these two enzymes are largely distinct.

To validate novel RNA editing sites identified by the RNA seq analysis of 293T/A3G cells, we performed Sanger sequencing of 24 new protein recoding C>U RNA editing sites in 24 genes (Table 3). These genes were chosen either because their editing levels were high enough (e.g. SCD, TM7SF3, CLASP1, PRPSAP2) to be informative in site-directed mutagenesis studies (below) or they were previously linked to HIV-1 infectivity (e.g. NMT1, CHMP4B, MPAK1) through functional studies. We validated RNA editing for 24 of 24 by Sanger sequencing in duplicate 293T/A3G transfectants, with possible exception of the ZNF142 editing which was seen only in only one replicate (FIG. 28B). With the exception of NMT1, RBM14, MED1 and MAPK1, Sanger validation experiments were performed in the same samples used in RNA seq analysis (Table 3).

To identify common features of sequence contexts of the editing sites, we examined 12 b-long sequences flanking the edited C residue. The edited C has a C, U, A or G at the immediate 5′ position for 613 (87%), 84 (12%), 9 and 6 sites, respectively. This observation and sequence logo analysis (FIG. 29C) suggests [CGU]N[CU]C[AG] as a sequence motif that is commonly targeted by APOBEC3G (The residues within brackets are different possibilities for a position. Edited C is underlined.). CCC, ACC and UCC are respectively seen for 190 (27%), 179 (25%) and 163 (23%) of the 712 editing sites. We previously noticed that edited Cs by A3A were frequently flanked by inverted repeats. Here, analysis of 25 nts containing the edited C in the middle for all edited sites by A3G shows that the edited C was flanked by a pair of inverted repeat sequences of 3-10 b for 699 (98%) of the editing sites. Sequences of 4 b are the most common, seen for 233 (32.7%) sites. Inverted repeat sequences of 4 bases or longer flanked over 75% of edited Cs by A3G, but only 28.6% in randomly obtained 25 nt sequences from human GRCh38 RefSeq transcriptome. GGC, GCC and GGCC are the three most common repeat sequences, seen for 11 (1.5%), 10 (1.4%) and 10 (1.4%) of sites, suggesting that flanking sequence complementarity rather than the sequence per se promotes editing activity. These analyses suggest that, similar to A3A, both sequence context, especially the immediate 5′-nucleotides, and the presence of long flanking inverted repeats play an important role in selection of edited Cs by A3G.

We previously noted that Cs edited by APOBEC3A are frequently located at the 3′-end of a tetra nucleotide flanked by long inverted repeats, suggesting a stem-loop structure. Here, examination of the Cs edited by APOBEC3G and confirmed by Sanger sequencing also suggests similar stem-loop structure. The Cs edited by APOBEC3G is located commonly at the 3′-end of a tetra loop flanked by inverted repeats (median=4) (Table 3).

Among the six transfectant samples of the study, 18,028 genes were considered as expressed and were analyzed for differential expression. Of the 7,582 (42.1%) genes that were differentially expressed (P<0.05), 61 and 83 were respectively down- and up-regulated with >2 fold-change in the APOBEC3G transfectants compared to controls.

In Vitro RNA Editing by Purified APOBEC3G

Transfection experiments that show editing of RNA but not DNA suggest that RNA is a substrate for A3G. To confirm that A3G can edit RNA in vitro, we generated 405 nt RNA sequence spanning nucleotides c.632-c.1036 of KIAA1715 by in vitro transcription and incubated it with APOBEC3G protein purified from overexpressing 293T cells. An 89 nt long ssDNA substrate containing the KIAA1715 cDNA sequence through nucleotides c.68-c.772 was included as a control. KIAA1715 mRNA acquires c.C751U mutation upon transient overexpression of A3G in 293T cells. As expected, APOBEC3G catalyzed c.C751U site-specific deamination in the RNA substrate. None of the other CC or TC sequences shows evidence of deamination by Sanger sequencing. C>T mutations were also not evident in the corresponding c.C751 site in the ssDNA template. Lack of deamination detectable by Sanger sequencing in ssDNA template is expected since A3G deaminates viral cDNA at 1%-1.5% levels which are distributed to multiple Cs {{729 Harris, Reuben S 2003; 730 Feng, Y. 2011;}}. These results suggest that certain sites in RNA are more favorable deamination substrates than ssDNA by A3G, likely owing to certain sequence/structural contexts in RNA.

A3G Site-Directed Mutagenesis for RNA Editing

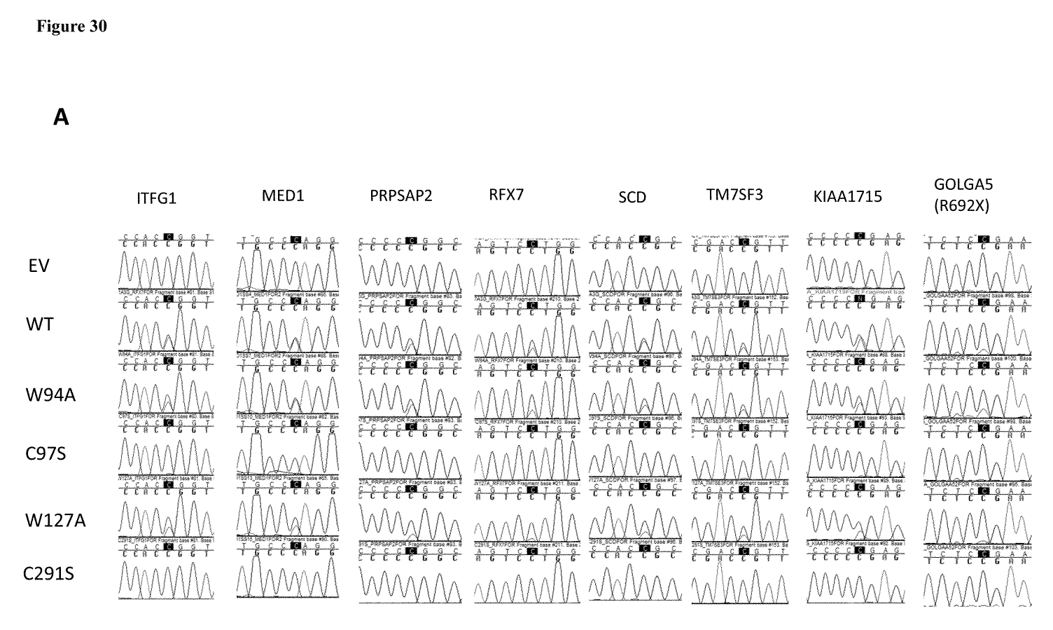

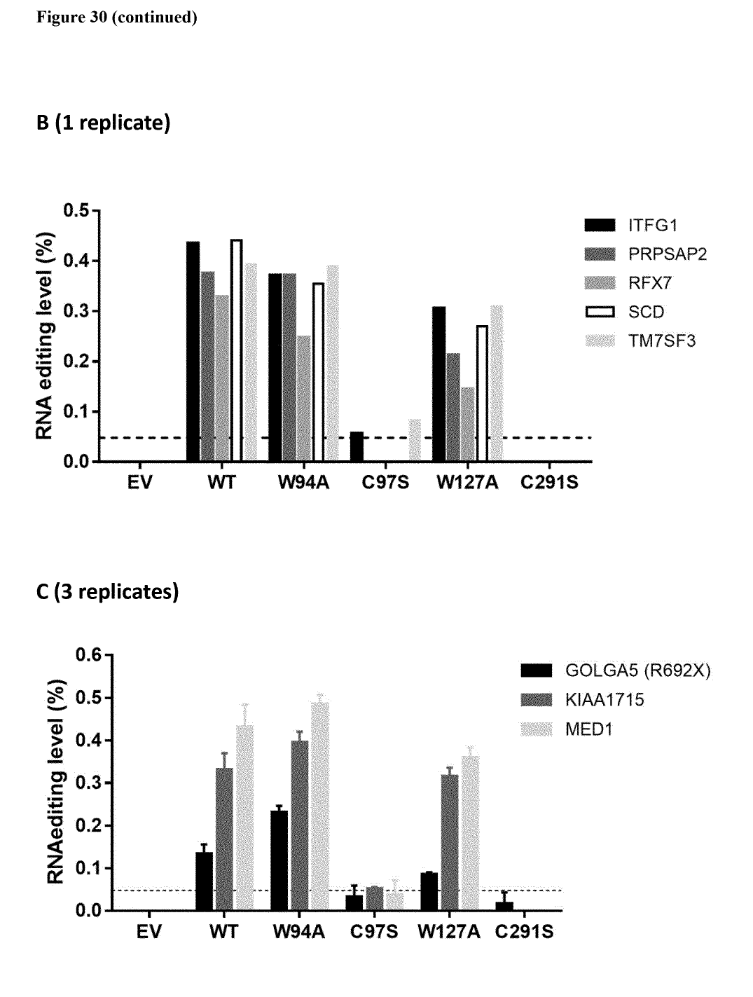

A3G NTD is involved in non-specific RNA binding but not in ssDNA deamination. To examine whether NTD is involved in RNA deamination, we initially created NTD core catalytic site mutant C97S, CTD core catalytic site mutant C291S, NTD critical RNA binding mutants W94A, W127A and C97S/C291S, W94A/W127A double mutants by site directed mutagenesis. Sanger sequencing of 293T/A3G transfectants of the mutants for eight highly edited genes showed that among single site mutants, the most dramatic reduction in RNA editing levels was observed by C97S and C291S mutants. C291S completely abolished RNA editing for all genes. C97S completely abolished RNA editing for MED1, GOLGA5 (C2074T), RFX7, PRPSAP2 and SCD but minimal residual editing was observed for ITFG1, KIAA1715 and TM7SF3. These results suggest that catalytic integrity of both NTD and CTD is essential for RNA editing, but certain RNA targets may be minimally edited even without an active NTD active site. W127A and to a lesser extent W94 mutants are reported to be essential for RNA interaction, oligomerization and virion encapsidation. We find that W127A, but not W94A, mutant impaired RNA editing (FIG. 30). Double mutant W127A/W97A abolished RNA editing completely, indicating that RNA binding by NTD is essential for cellular RNA editing.

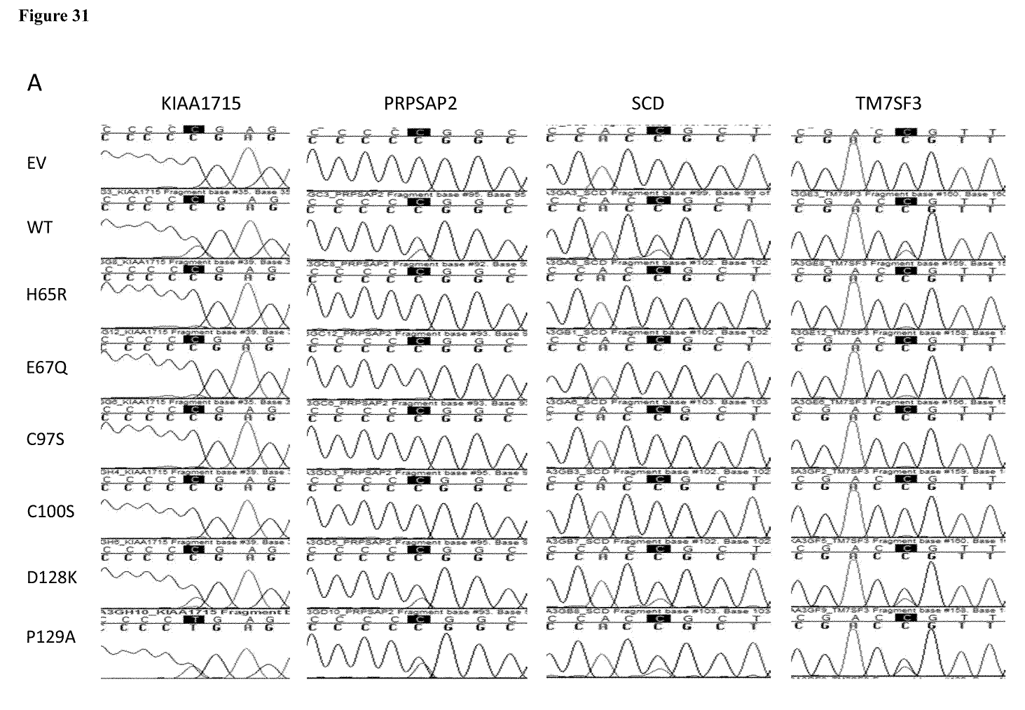

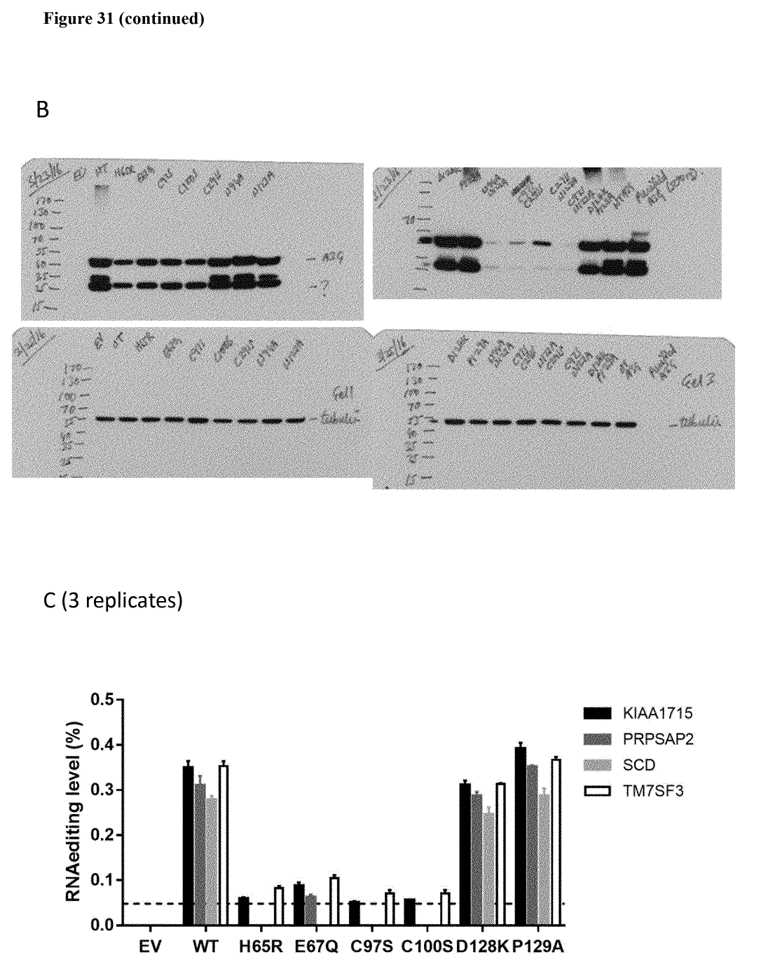

To further examine role of the NTD conserved catalytic domain residues, we created additional mutants C100S, H65R, E67Q, D128K and P129A. These results show that N-terminal conserved catalytic residues C100, H65R and E67Q are essential for normal RNA editing (FIG. 31). D128 and P129 residues have no role on RNA editing. The site-directed mutagenesis data indicate that both N and C terminal catalytic domains are required for RNA editing.

In Vitro RNA Editing by Purified APOBEC3G

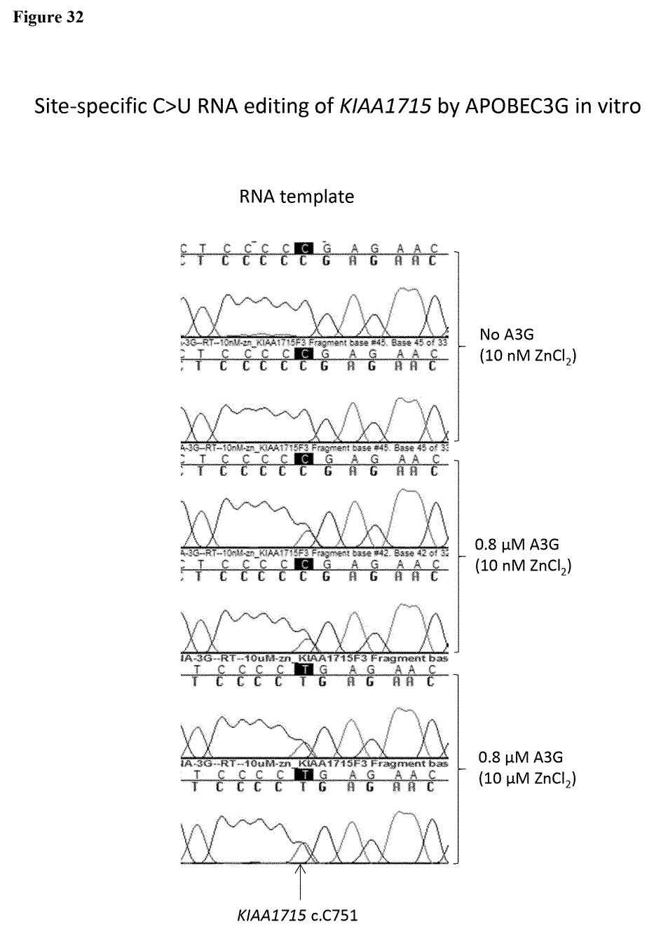

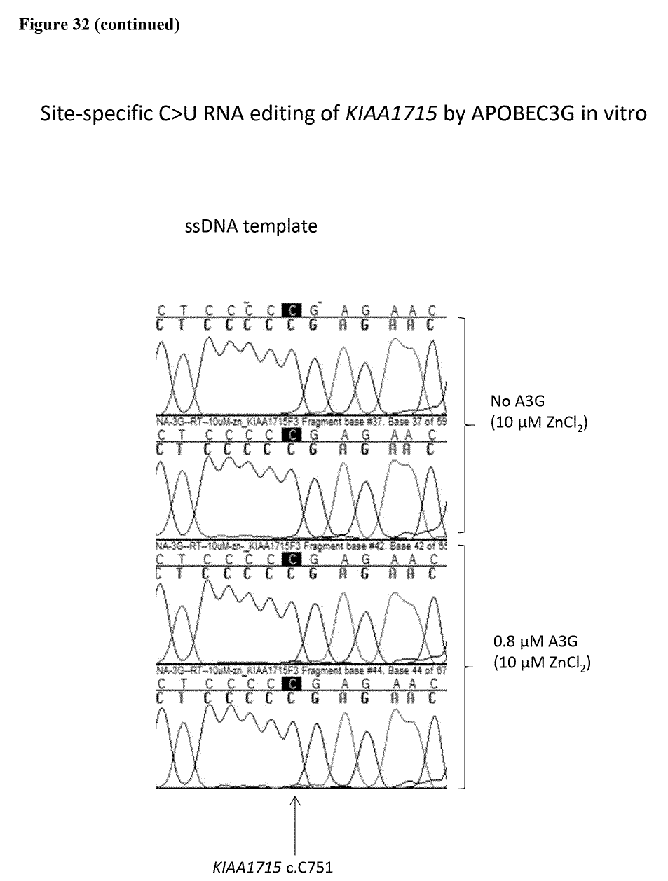

Transfection experiments that show editing of RNA but not DNA suggest that RNA is a substrate for A3G. To confirm that A3G can edit RNA in vitro, we generated 405 nt RNA sequence spanning nucleotides c.632-c.1036 of KIAA1715 by in vitro transcription and incubated it with APOBEC3G protein purified from overexpressing 293T cells. An 89 nt long ssDNA substrate containing the KIAA1715 cDNA sequence through nucleotides c.68-c.772 was included as a control. KIAA1715 mRNA acquires c.C751U mutation upon transient overexpression of A3G in 293T cells. APOBEC3G catalyzed c.C751U site-specific deamination in the RNA substrate (FIG. 32). None of the other CC or TC sequences shows evidence of deamination by Sanger sequencing. C>T mutations were also not evident in the corresponding c.C751 site in the ssDNA template. Lack of deamination detectable by Sanger sequencing in ssDNA template is expected since A3G deaminates viral cDNA at 1%-1.5% levels which are distributed to multiple Cs. These results suggest that certain sites in RNA are more favorable deamination substrates than ssDNA by A3G, likely owing to certain sequence/structural contexts in RNA.

RNA Editing by APOBEC3G Targets Cellular Genes Involved in HIV-1 Infection

We noted that some host genes known to be involved in HIV infectivity are targeted for RNA editing. These genes include ACIN1, CHMP4B, SIN3A, subunit genes of mediator complex MED (MED1, MED28), NFAT5, NMT1, RBM14 and MAPK1. MED1, which encodes a subunit of SP1/mediator complex is implicated in HIV-1 replication in several studies. Other edited genes that belong to cellular pathways important for HIV-1 infectivity (e.g. NF-KB, ESCRT, chromatin modifications) are also noted (Table 4). RNA editing of host genes linked to HIV-1 infectivity by previous functional studies suggests that A3G may alter the host environment to antagonize HIV-1 infection. The antagonistic effect of RNA editing may involve reducing the amount or quality of the accessory host proteins that are critical for HIV-1 life cycle. Alternatively, but not exclusively, RNA editing of host genes might facilitate virion encapsidation of A3G by modifying the intracellular protein trafficking pathways.

TABLE 2
Gene features and effects on translation codon for
APOBEC3G-mediated C > U RNA editing sites^{a}

| 5′ untranslated region | | | 39 |
|---|---|---|---|
| Exonic | Synonymous | | 174 |
| | Non-synonymous | Nonsense | 48 |
| | | Stop loss | 0 |
| | | Missense | 173 |
| | | Unknown | 2 |
| | Non-coding RNA | | 8 |

| 3′ untranslated region | | 227 |
|---|---|---|
| Intronic | Coding RNA | 12 |
| | Non-coding RNA | 7 |

| | |
|---|---|
| Untranscribed^{b} | 10 |
| Intergenic | 12 |

^{a}As reported by the ANNOVAR annotation tool
^{b}Within 1 kb up- or down-stream respectively of a known transcription start or end site

TABLE 3
Sanger validation of selected C>U recoding RNA editing sites identified in 293T/A3G cells

| | | | RNA | | Sizes (of loop |
|---|---|---|---|---|---|
| | | | editing | RNA | (N..C)/ |
| | | | level | editing | immediately |
| | Chromosomal | | (RNA | level | flanking |
| Gene^{a} | position^{b} | cDNA and amino acid change^{c} | seq) | (Sanger) | palindrome^{d} |
| AGIN1 | 14: | NM_001164816:exon6:c.C676T:p.Q226X | 0.22 | 0.23 | 4/5 |
| | 23063490 | | | | |
| CDC6 | 17: 40291480 | NM_001254:exon4:c.C472T:p.Q158X | 0.08 | 0.14 | 3/6 |
| CHMP4B | 20: | NM_176812:exon3:c.C412T:p.Q138X | 0.07 | 0.09 | 4/5 |
| | 33850995 | | | | |
| CLASP1 | 2: | NM_001142273:exon9:c.C829T:p.R277W | 0.26 | 0.45 | 3, 4/2 |
| | 121469844 | | | | |
| GOLGA5 | 14: | NM_005113:exon4:c.C937T:p.Q313X | 0.09 | 0.19 | 4/4 |
| | 92809464 | | | | |
| GOLGA5 | 14: | NM_005113:exon12:c.C2074T:p.R692X | 0.10 | 0.13 | 3/3 |
| | 92837408 | | | | |
| ITFG1 | 16: | NM_030790:exon18:c.C1816T:p.R606W | 0.16 | 0.38 | 3/2 |
| | 47155742 | | | | |
| KIAA1715 | 2: | NM_030650:exon10:c.C751T:p.R251X | 0.26 | 0.34 | 4/5 |
| | 175939613 | | | | |
| MAPK1 | 22: | NM_002745:exon6:c.C740T:p.P247L | 0.13 | 0.11 | 4/3 |
| | 21788373 | | | | |
| MED1 | 17: | NM_004774:exon17:c.C1963T:p.Q655X | 0.27 | 0.38 | 4/6 |
| | 39410258 | | | | |
| NFAT5 | 16: | NM_006599:exon12:c.C3389T:p.S1130L | 0.12 | 0.12 | 4/2 |
| | 69693268 | | | | |
| NFRKB | 11: | NM_006165:exon20:c.C2527T:p.Q843X | 0.07 | 0.09 | 7/5 |
| | 129873843 | | | | |
| NMT1 | 17: | NM_021079:exon1:c.C44T:p.P15L | 0.23 | 0.19 | 4/6 (i + 1) |
| | 45061373 | | | | |
| NVL | 1: | NM_001243146:exon11:c.C796T:p.Q266X | 0.06 | 0.09 | 4/4 |
| | 224289696 | | | | |
| PRPSAP2 | 17: | NM_001243936:exon9:c.C802T:p.R268W | 0.25 | 0.35 | 4/4 |
| | 18928928 | | | | |
| RBM14 | 11: | NM_006328:exon3:c.C1846T:p.R616C | 0.22 | 0.26 | 4/2 |
| | 66626504 | | | | |
| RFX7 | 15: | NM_022841:exon9:c.C1256T:p.P419L | 0.29 | 0.29 | 4/3 |
| | 56096472 | | | | |
| SCD | 10: | NM_005063:exon3:c.C376T:p.R126C | 0.24 | 0.33 | 4/4 |
| | 100352431 | | | | |
| SGPL1 | 10: | NM_003901:exon5:c.C355T:p.Q119X | 0.05 | 0.11 | 4/4 (i + 1) |
| | 70854801 | | | | |
| SUCLA2 | 13: | NM_003850:exon5:c.C661T:p.Q221X | 0.13 | 0.17 | 4/6 |
| | 47973266 | | | | |
| TM7SF3 | 12: | NM_016551:exon12:c.C1529T:p.P510L | 0.30 | 0.39 | 4/2 |
| | 26974149 | | | | |

TABLE 4
Genes that undergo C > U recoding RNA editing by APOBEC3G regulate
cellular pathways involved in HIV-1 infection.

| | | | | Interacts | | | | | | |
|---|---|---|---|---|---|---|---|---|---|---|
| ESCRT | RNA, | | | with | | | | | | |
| pathway | DNA | | | HIV | Nuclear | | | | | |
| HIV RNA | replication, | NF-KB | Chromatin | RNA/ | membrane/ | | | | MAPK | |
| trafficking | modification | pathway | modifiers | proteins | pore | Cytoskeleton | Mitochondria | Proteasome | signaling | Golgi |
| ZNF142 | MED1, | USP34 | PAXIP1 | ACIN1 | LBR | PDLIM3 | DNM1L | PSMD4 | LAMTO | GOL |
| | MED15, | | | | | | | PSMC3 | R3 | GA3, |
| | MED28 | | | | | | | | | GOL |
| | | | | | | | | | | GA5 |
| CHMP4B | PAPOLG | TAB1 | NFRKB | WDR48 | NUP205 | CLASP1 | SUCLA2 | | MAPK1 | |
| MID2 | CDC6 | UBE2L3 | CTCF | NSUN5 | NUP54 | CAMSAP1 | | | | |
| RBM14 | SETD2 | UBE2N | SMARCA4 | NMT1 | | | | | | |
| | | (UBC13) | | | | | | | | |
| VPS37A | NFAT5 | | KMT2A, | HSPA5 | | | | | | |
| | | | KMT2C, | | | | | | | |
| | | | KMT2D | | | | | | | |
| ATRN | NEIL3 | | BAZ1B | | | | | | | |
| AP2M1 | HUWE1 | | CHD7, | | | | | | | |
| | | | CHD8 | | | | | | | |
| SIN3A | EIF3I | | CBX6 | | | | | | | |
| SMG6 | | | ARID4A | | | | | | | |
| | | | PHF2 | | | | | | | |
| | | | ATF7IP | | | | | | | |
| | | | JADE1 | | | | | | | |
| | | | ASH1L | | | | | | | |
| | | | EP300 | | | | | | | |

What is claimed is:

1. A method for identifying agents that enhance or inhibit C>U deamination in a RNA molecule comprising:
a) providing a RNA substrate which contains a motif that contains a C that can undergo deamination to U;
b) contacting the RNA substrate with a apolipoprotein B editing catalytic (APOBEC) protein in the presence or absence of test agents;
c) determining the extent of C>U deamination and identifying agents in the presence of which either an increase or decrease of deamination is observed as compared to deamination in the absence of the agent,
Wherein, the APOBEC protein is APOBEC3A or APOBEC3G.

2. The method of claim 1, wherein the motif is CCAUCG.

3. The method of claim 1, wherein the APOBEC3A or APOBEC3G is a purified protein.

4. The method of claim 1, wherein the APOBEC3A or APOBEC3G is a recombinant protein.

5. The method of claim 1, wherein the APOBEC3A or APOBEC3G is in a cell lysate.

6. A method for identifying agents that enhance or inhibit C>U deamination in a RNA substrate comprising:
a) providing cells which express apolipoprotein B editing catalytic 3A (APOBEC3A);
b) in the presence or absence of test agents, exposing the cells to conditions under which the cells can carry out APOBEC3A driven C>U deamination of RNA; and
c) determining the extent of C>U deamination in RNA to identify agents that induce or inhibit C>U deamination in RNA,
wherein an increase in C>U deamination as compared to deamination in the absence of the agent identifies an agent that enhances C>U deamination, and a decrease in C>U deamination as compared to deamination in the absence of the agent identifies an agent that inhibits C>U deamination.

7. The method of claim 6, wherein the cells are monocytes, and the condition under which the cells carry out APOBEC3A driven C>U deamination of RNA comprise hypoxia, exposure to interferon or both.

8. The method of claim 7, wherein the interferon in type 1 interferon or interferon gamma.

9. The method of claim 5, wherein the cells are macrophages, and the condition under which the cells carry out APOBEC3A driven C>U deamination of RNA comprises exposure to interferon.

10. The method of claim 9, wherein the interferon in type 1 interferon or interferon gamma.

11. A method for identifying agents that enhance or inhibit C>U deamination in a RNA substrate comprising:
a) providing cells which have been transfected to overexpress apolipoprotein B editing catalytic3A (APOBEC3A) or apolipoprotein B editing catalytic 3G (APOBEC3G);
b) in the presence or absence of test agents, determining the extent of C>U deamination in RNA to identify agents that enhance or inhibit C>U deamination in RNA.

12. The method of claim 11, wherein the cells are 293T cells.
